(19) United States
(12) Patent Application Publication
(10) Pub. No.: US 2007/0072201 A1
(43) Pub. Date: Mar. 29, 2007

(54) METHOD FOR PRODUCING IMPROVED NUCLEIC ACID OLIGOMER FUNCTIONAL HOMOGENEITY AND FUNCTIONAL CHARACTERISTIC INFORMATION AND RESULTS AND OLIGOMER APPLICATION RESULTS

(76) Inventor: David E. Kohne, San Diego, CA (US)

Correspondence Address: WESLEY B. AMES, 7031 LOS VIENTOS SERENOS, ESCONDIDO, CA, 92029, US

(21) Appl. No.: 11/383,203

(22) Filed: May 13, 2006

Related U.S. Application Data
(60) Provisional application No. 60/681,524, filed on May 16, 2005.
(60) Provisional application No. 60/681,426, filed on May 16, 2005.

(51) Int. Cl. C12Q 1/68; C12P 19/34

(52) U.S. Cl. 435/6; 435/91.2

(57) ABSTRACT

An approach and methods are provided for obtaining improved information and results concerning the functional homogeneity and functional characteristics of a chemically synthesized or biologically synthesized nucleic acid oligomer of any type, and for obtaining improved information and results concerning the functional homogeneity and functional characteristics of the oligomer under the conditions of the oligomer application, and for obtaining improved results for the oligomer application, and for obtaining improved results for any application which utilizes such improved oligomer application results.

### RELATED APPLICATIONS

This application claims the benefit of Kohne, U.S. Provisional Application 60/681,426, filed May 16, 2005, and of Kohne, U.S. Provisional Application 60/681,524, filed May 16, 2005, each of which is hereby incorporated by reference in its entirety.

### FIELD OF THE INVENTION

The present invention relates to nucleic acid oligomers, and to method for improving functional homogeneity of such oligomers.

### BACKGROUND OF THE INVENTION

The following discussion is provided solely to assist the understanding of the reader, and does not constitute an admission that any of the information discussed or references cited constitute prior art to the present invention.

Quality and Purity of Prior Art Produced Nucleic Acid Oligomers.

Natural and modified nucleic acid oligomers can be produced by chemical synthesis or by in vitro enzymatic or biological means (1,2,3,4). Such RNA and DNA and modified oligomer nucleic acid molecule production is widespread and routine. Herein, nucleic acid molecules of up to 400 nucleotides in length will be termed nucleic acid oligomers. Currently oligomer molecules with a nucleotide length N of 150 nucleotides or so can be produced by chemical synthesis. Nucleic acid molecules with N values in the thousands are routinely produced by biological and in vitro enzymatic synthesis. Unmodified or natural oligomer molecules are composed of the biologically common natural ribo- and deoxyribo-nucleotides. Modified oligomer molecules are composed of one or more or all modified nucleotides, which do not occur commonly, or at all, in nature.

The vast majority of chemically synthesized oligomers of any kind are produced using an automated nucleic acid synthesizer instrument, which separately produces multiple oligomers, all at one time. For each separate oligomer synthesis, the instrument is programmed to synthesize a desired oligomer, which is intended to have a particular known nucleotide sequence, and known nucleotide length. After synthesis, the synthesized oligomer preparation is recovered after removing protective and other chemical groups, which are associated with the synthetic process.

Ideally, the recovered synthesized product oligomer preparation should consist of a population of oligomer molecules, which are identical to one another. That is, ideally all of the resulting synthetic oligomer molecules should represent the intended oligomer molecule and should be identical to one another in nucleotide sequence, nucleotide length, nucleotide composition and physical-chemical properties. In other words, in the ideal situation all individual oligomer molecules present in the synthesized oligomer population will have identical physical-chemical characteristics, and the synthetic oligomer preparation is composed of a homogeneous population of synthetic oligomer molecules of the intended nucleotide length N.

However, in reality this ideal situation does not occur for synthesized oligomers. It is well known that prior art nucleic acid synthesis practices of all kinds essentially always produce chemically synthesized oligomer molecule populations in which oligomer molecules which have different nucleotide sequences, different nucleotide lengths, different nucleotide compositions, and different physical-chemical properties, are present in significant amounts (5-11). It is not uncommon for one-half or more of the chemically synthesized and recovered molecules to be imperfect with regard to the intended oligomer molecule. Such oligomer molecules have different nucleotide sequences, lengths and compositions, and different physical-chemical properties than the intended oligomer molecules. Such prior art non-homogeneous preparations of synthetic oligomers are often used for a variety of purposes, such as PCR primers, capture oligomers for spotting on microarrays, primers for other non-PCR nucleic acid amplification, and other purposes. Such use of unpurified synthesized oligomers for an application does not produce optimal performance or results for the application.

For many prior art applications, the art recognizes that the use of the unpurified heterogeneous oligomer molecules produces unacceptable suboptimal performance and/or results for many prior art applications. Prior art recognizes that the oligomers which have the desired and intended nucleotide length N, nucleotide sequence, and nucleotide composition, and therefore the intended physical-chemical properties, will provide optimal application performance and results. In an effort to obtain such intended oligomer molecules from the unacceptable unpurified or crude heterogeneous oligomer prep, prior art methods fractionate the unpurified oligomers and isolate an oligomer fraction which is greatly enriched for oligomer molecules which have the intended nucleotide length N (1-5,11-13). Prior art routinely produces such purified oligomer preps, which consist of 90-95% or more N oligomers. This enrichment improves the effectiveness of the oligomer in the oligomer application and improves the utility of the oligomer and the oligomer application. For such purified N oligomer molecule preps, prior art believes that a purified N oligomer molecule population consists of oligomer molecules with the intended nucleotide sequence and nucleotide composition, and therefore the intended physical-chemical properties.

A wide variety of methods are utilized to produce such purified N oligomer preps. The most widely used methods are purification and fractionation by gel electrophoresis and HPLC methods, including hydrophobic and ion exchange HPLC methods. Each of these methods relies primarily on a particular physical-chemical property as the primary basis for separating the different oligomers from each other and purifying the separated fractions. It appears that hydrophobic HPLC methods are not effective at discriminating small differences in oligomer ionic charge, and therefore small differences in oligomer nucleotide length. However, HPLC ion exchange methods are effective in detecting single nucleotide length differences between oligomers, for oligomers up to about 50 nucleotides long. Capillary gel electrophoresis and gel electrophoresis also separates oligomers on the basis of ionic charge and can also detect a single nucleotide difference in oligomer length. Such information and methods are well known in the prior art.

Prior art has developed a variety of well known analytical methodologies for analyzing and quantitating certain aspects of the quality and purity of synthetic oligomers. Such methods include hydrophobic and ion exchange HPLC methods, capillary gel electrophoresis methods, gel electrophoresis and chromatography methods, and mass spectrometry methods. Each of these methods relies primarily on a particular physical-chemical property of the oligomer molecules for its analytical and quantitative capabilities. It appears that the hydrophobic HPLC methods are not effective at discriminating small differences in nucleotide length, while methods, which rely on separation and quantitation of oligomers on the basis of charge can be used to detect and quantitate small differences in oligomer length. Mass spectroscopy provides the most sensitive method for discriminating small differences in oligomer nucleotide length or mass. Small differences in mass and single nucleotide differences between oligomers can be detected between oligomers of up to about 100-120 nucleotides length. For a mass spectroscopy analysis, the primary basis for oligomer separation and fractionation is the magnitude of the ionic charge/mass ratio difference for the oligomers. As described above, the most effective methods for both the quality control analysis of a synthesized oligomer prep, and for enrichment and purification of the desired oligomer molecules from the rest, rely primarily on the ability to detect and separate oligomers on the basis of differences in oligomer ionic charge, and therefore are essentially based on the ability to separate oligomers of different nucleotide length. Such methods are not generally effective for determining oligomer differences, which are not charge related. In certain cases, mass spectroscopy and gel electrophoresis and gel capillary electrophoresis can detect some non-charge related differences in oligomers, but these methods are not known to detect all such non-charge related differences. The above-discussed prior art methods are limited in their ability to detect and remove all known oligomer imperfections.

It appears that prior art separation and purification methods can routinely produce purified enriched synthetic oligomer preps which are comprised of oligomer nucleotide molecules of only the intended programmed nucleotide length. Herein, the programmed nucleotide length is referred to as N. A shorter oligomer in the same oligomer prep will be termed an N−X oligomer molecule, where X equals the nucleotide difference between the N oligomer and the shorter oligomer. A longer oligomer in the same oligomer prep will be termed an N+X oligomer molecule, where X equals the nucleotide difference between the N and the longer oligomer. Thus, it appears that prior art routinely produces purified populations of oligomer molecules in which 90-95 percent or more of the molecules present are full sized oligomer molecules with a nucleotide length of N.

While prior art can demonstrate that these oligomer molecules have the same N nucleotide length, it cannot and does not know whether the population of N oligomer molecules is a homogeneous population of oligomer molecules which have the intended nucleotide sequence and nucleotide composition, and therefore the intended physical-chemical properties or not. All that is known is that these N oligomer molecules are measured to have the same nucleotide length. Prior art does not determine whether such a purified N oligomer molecule population is composed of oligomer molecules, which have identical physical-chemical properties, or whether the oligomer molecules have the intended physical-chemical properties. Note that nucleic acid sequencing of the oligomer prep will not determine this. This occurs because the sequence determined represents an average sequence, and can be used to determine whether the oligomer molecules are heterogeneous only when the oligomer molecule population is heterogeneous in particular ways. The oligomer population can be significantly heterogeneous in other ways, which are not detected by sequencing.

As an example, consider the following oligomer prep situation. (i) A DNA synthesizer is programmed to produce an N=50 oligomer with a particular DNA sequence. (ii) The resulting synthesized oligomer prep is purified and the purified prep contains only N=50 oligomers. (iii) During synthesis, a wrong nucleotide is randomly incorporated into a growing oligomer with a frequency of 1 out of 50. As a result, on average there is one wrong base associated with each N=50 oligomer molecule in the purified oligomer prep. Since the nucleotide errors occur randomly at each nucleotide position of the oligomer, one out of 50 oligomer molecules will possess a wrong base at a particular nucleotide sequence position. Thus, for any particular nucleotide sequence position in the oligomer, two percent of the N=50 oligomer molecules will be associated with a wrong base. (iv) In this situation prior art sequencing methods cannot detect this level of oligomer sequence heterogeneity. Further, for this oligomer heterogeneity model the magnitude of oligomer sequence heterogeneity must be much greater to be detectable by standard prior art sequencing methods.

The vast majority of synthetic oligomers of all kinds are designed and produced to be used in one or another application which requires an oligomer to specifically recognize a particular nucleotide sequence in a complementary target nucleic acid molecule, and then to hybridize with the said complementary target molecule to form a stable oligomer-target duplex. Thus, at a minimum, an intended and desired function of the vast majority of all prior art produced synthetic oligomer prep molecules of all kinds is to specifically recognize the complementary nucleotide sequence of, and stably hybridize with, a particular target molecule. For the vast majority of prior art synthetic oligomer applications and uses, prior art generally tacitly believes that the results of the application or use of each oligomer are optimal when the following conditions are met. (a) All or essentially all of the N oligomer molecules in the oligomer prep can stably hybridize with the intended target nucleic acid molecule. In other words, when the synthetic oligomer is hybridized to an equal or greater mole amount of the complementary target molecules, essentially all of the oligomer molecules form stable oligomer-target duplexes. (b) For each stably hybridized oligomer-target duplex molecule, the intended and desired and designed duplex region consists of only the intended nucleotide pairs. Note that the intended nucleotide pairs are almost always perfectly complementary nucleotide pairs, but that an oligomer molecule may be designed so that a mismatched or unpaired nucleotide occurs at an intended sequence position in the oligomer-target duplex. These conditions can be met only if essentially all of the oligomer molecules in a synthetic oligomer prep have the same intended nucleotide length, and the same intended nucleotide sequence and nucleotide composition. In other words, these conditions can be met only if the synthetic oligomer prep consists of an essentially homogeneous population of oligomer molecules, which have the intended physical-chemical properties.

Prior art produced synthetic oligomer preps of all kinds are routinely produced by a large variety of commercial and non-commercial sources. Such oligomer preps are often synthesized, deprotected, and recovered, and used without further purification or characterization. Other such oligomer preps are further purified and characterized as discussed earlier. However, the functional properties or characteristics of these prior art produced synthetic oligomer preps are only very rarely even partially evaluated by the manufacturer or end user before being utilized for their designed and intended application. Even for those rare instances where the oligomer prep functional properties are partially evaluated, the methods used by the prior art to evaluate the oligomer prep functional properties are limited in their ability to correctly characterize key aspects of the synthetic oligomer prep molecules functional properties.

The prior art approaches commonly used for such N oligomer prep functional characterization are inadequate in at least the following ways. (i) The methods do not determine the maximum extent to which the synthetic N oligomer prep molecules can stably hybridize with the intended target nucleic acid molecules. (ii) The methods do not determine whether the purified N oligomer molecule population is actually a homogeneous population of N oligomer molecules which have the same nucleotide sequence and nucleotide composition, and therefore the same physical-chemical properties, or not. In other words, the prior art methods do not determine whether the purified N oligomer molecule population is functionally homogeneous. (iii) The methods do not determine whether the purified N molecule population consists of oligomer molecules, which have the intended nucleotide sequence and intended nucleotide composition, and therefore the intended chemical-physical properties. In other words, the methods do not determine whether the purified N oligomer molecule has the intended functional homogeneity. These issues are discussed below.

The prior art methods commonly used for such functional characterization are not useful for detecting even moderate heterogeneity which may be present in the synthetic oligomer molecule population analyzed. The most commonly used method for prior art characterization of the functionality of an oligomer prep is the well-known optical melt method (5,14,15). Herein, the optical melt method will be termed the OM. The OM is designed and used to determine the optically measured thermal melting characteristics of the hybridized double strand oligomer duplex. Such an analysis generally involves the following. (i) Separately produce and purify as desired the oligomer of interest and its synthetic oligomer perfect complement. (ii) Mix known equimolar amounts of the oligomer of interest and the complement oligomer into the desired melting buffer solution. Almost always a final concentration of around 10⁻⁶M oligomer is necessary in order to be able to detect the presence of the oligomers. (iii) Place the mixture into a thermal stability measurement instrument at a temperature so that the complementary oligomers hybridize to completion to form the best possible helical duplex molecules. The solution containing oligomer duplex molecules has a lower absorbance than the solution where both oligomers are in a single strand state. (iv) The instrument is programmed to continuously but slowly raise the temperature of the duplex containing solution, and at the same time to monitor the absorbance of the solution. When the temperature becomes high enough the oligomer duplexes will dissociate from each other to become single stranded, and at the same time the absorbance of the solution will increase until all of the duplexes are dissociated. (v) The temperature at which one-half of the maximum absorbance increase occurs for the solution is used to characterize the melting characteristics of the oligomer duplexes. This temperature is commonly termed the Tm. The Tm of the oligomer duplex is dependent on a variety of factors including the melting solution composition, the oligomer duplex nucleotide length, nucleotide sequence, and nucleotide composition, and the molar concentration of the oligomers in the melting solution. At the Tm the rate of oligomer duplex dissociation equals the rate of oligomer hybridization or association, and prior art believes that at the Tm the oligomer duplex and single strand states are at equilibrium.

Such prior art OM oligomer analyses indicate the following concerning the oligomer functional characteristics. (a) A significant fraction of the oligomer of interest is capable of hybridizing with a complementary oligomer nucleotide sequence. This indicates that the oligomer is significantly specific for the complementary oligomer to form helical duplexes, which exhibit reduced absorbance. However, the OM analysis results do not indicate whether the oligomer of interest is a homogeneous population of oligomer molecules or not. Further, the OM analysis results do not indicate whether the oligomer of interest can hybridize completely with the complementary oligomer or not. Given further information, which prior art does not measure or provide, a rough estimate of the extent of hybridization and the homogeneity of the oligomer of interest could be made. It is likely that most such prior art OM analyzed oligomers of interest hybridize to 70-90 percent extent or more with the complementary oligomer. In addition, the significance of the measured Tm value for an oligomer OM analysis cannot be known, absent further information which is not provided or known by the prior art. Absent such knowledge it cannot be known whether the measured Tm value reflects the Tm value for perfectly base pair matched oligomer duplexes, or imperfectly matched oligomer duplexes. Further, it cannot be known whether the analyzed oligomer duplex molecule population is composed of a mixture of perfect match and imperfect match oligomer duplexes. This commonly used OM approach then, provides only limited information concerning the functional properties of the oligomer of interest, and provides only limited information concerning the homogeneity or non-homogeneity of the oligomer of interest.

An OM derived oligomer Tm value is often used by the prior art in an effort to rationally design and predict the duplex thermal stability or duplex dissociation parameters for an application, which uses oligomer molecules (17). Such applications include mutation detection tests, and oligomer probe based diagnostic tests of all kinds, and oligomer based capture probes of all kinds. Virtually all of these and other applications utilize oligomers at a concentration where the oligomer duplex dissociation temperature is not oligomer concentration dependent. Such a non-oligomer concentration dependent oligomer duplex dissociation temperature is herein termed a half dissociation temperature, or a T.5d. For an oligomer duplex analyzed at the same ionic strength and pH, the OM Tm value is almost always significantly higher than the T.5d value.

Prior art often uses oligomer OM analysis to generate values for certain thermodynamic parameters under different conditions (14-17). These thermodynamic parameter values are then widely used by the prior art to design oligomers for an intended oligomer application (18-20). A variety of commercial and other software programs incorporate these thermodynamic parameter values for use in designing oligomers for intended oligomer applications and for evaluating and designing nucleic acid inter- and intra-strand structure. Prior art believes and practices that such OM analysis derived thermodynamic parameter values are correct. In order for this prior art belief and practice to be valid, each OM analyzed oligomer prep used to produce the thermodynamic parameter values must be composed of an essentially homogeneous population of oligomer molecules which all have the same physical-chemical properties. This requirement for oligomer homogeneity is necessary in order for the equilibrium constant determined for the oligomer duplex preparation at the OM measured Tm to be correct. In order to derive a correct equilibrium constant value from the Tm analysis the analyzed oligomer duplex molecule population must be essentially homogeneous. As discussed, it cannot be known whether such prior art analyzed oligomer duplex molecule populations are homogeneous or not. Therefore, in order for prior art to believe and practice the correctness of the OM analysis thermodynamic parameter values, prior art tacitly assumes the analyzed oligomer duplex preps are essentially homogeneous.

Prior art basic research practice has for many years utilized one or another version of the OM analysis to characterize certain thermodynamic parameters which are associated with natural and modified RNA, DNA, and other oligomer duplexes of various nucleotide sequences, compositions and lengths, as well as varying degrees of complementarity. Such prior art studies tacitly assumed that essentially all of the oligomer molecules in an analyzed oligomer prep were identical in their physical-chemical properties, and that the oligomer prep molecule population was essentially homogeneous. Such prior art OM studies have the same limitations as discussed above, concerning the interpretation of the extent of hybridization and homogeneity of the analyzed oligomer population, and the Tm derived equilibrium constant.

Prior art has also used one or another OM methods in conjunction with other spectroscopic methods in order to determine the hybridization or association kinetics of complementary oligomers, and a measure of the melting or dissociation kinetics of the hybridized oligomer duplexes (21-24). These methods tacitly assumed that the analyzed oligomer preps were essentially homogeneous populations of the intended oligomer molecules. These studies have essentially the same limitations as discussed above, with regard to determining an analyzed oligomer prep's hybridization extent and homogeneity. These studies reported the following. (a) The kinetics of oligomer duplex dissociation is much more temperature dependent than the kinetics of complementary oligomer hybridization or association. It has been reported that the complementary oligomer hybridization kinetics show little temperature dependence until the Tm is neared. (b) The complementary oligomer hybridization or association kinetics show a much greater salt concentration dependence than does the oligomer duplex dissociation kinetics.

Prior art has also utilized non-spectroscopic analysis methods to partially characterize the functionality of synthetic oligomer preps (9,12,13,20,26,27,28). Such methods include various nuclease based hybridization analysis methods, various gel separation and electrophoresis methods, various filter and other immobilized material based hybridization analysis methods, and combinations of these methods. All of these prior art functional characterizations have essentially the same limitations as discussed above, with regard to determining an analyzed oligomer preps hybridization extent and homogeneity.

A significant fraction of virtually every prior art produced unfractionated oligomer prep is known to consist of synthesized oligomer molecules which are different from the intended oligomer molecule. The origin of many of the differences is well known. As discussed above, prior art has developed a variety of methods for detecting such different oligomer molecules, and removing them from the oligomer prep. Such methods involve purifying the synthesized oligomer prep so that the oligomer prep is greatly enriched for the oligomers, which have the intended N nucleotide length. Commercial oligomer producers generally represent that their highly purified synthetic oligomer preps consist of 90-95% or more of the intended N molecules. Such purification adds considerable expense and effort to the process of producing an oligomer prep. These purified synthetic oligomer preps are considered by the prior art to have the highest quality possible, and prior art generally believes and practices that such highly purified oligomer preps contain 90-95% or more of the intended oligomer molecules.

The underlying basis for this prior art belief and practice is twofold. First, all or essentially all of the oligomer molecules have the intended nucleotide length N. Second, faith in the nucleic acid synthesis process and lack of evidence to the contrary. Said prior art belief and practice can be valid only if the following is true. (a) Essentially all of the synthetic oligomers in the purified oligomer prep have the intended nucleotide length N. (b) The purified synthetic N oligomer prep molecules hybridize essentially completely with the intended target nucleic acid molecules. (c) The resulting N oligomer duplex molecule population consists of a homogeneous population of duplex molecules which have the intended physical-chemical properties and which have the intended functional homogeneity. Condition (a) appears to be met routinely for prior art purified synthetic oligomer fractions. Prior art does not determine or provide information, which addresses condition (b) or (c). Therefore, the prior art belief and practice that the prior art produced purified synthetic oligomer preps which are comprised almost exclusively of oligomer molecules of the intended nucleotide length N, have the intended nucleotide sequence and intended nucleotide composition and, therefore, have the intended physical-chemical properties and the intended functional homogeneity, cannot be known to be valid or invalid. In other words, it cannot be known whether such prior art purified oligomer preps consist of an essentially homogeneous population of oligomers or not. Further, it cannot be known whether the purified oligomer molecules consist of essentially only one nucleotide sequence and one nucleotide composition, or not. In addition, it cannot be known whether the physical-chemical characteristics and functional characteristics of the purified oligomer molecules are the same as those associated with oligomer molecules which have the intended nucleotide sequence, nucleotide composition, and nucleotide length.

Overall then, for prior art produced and purified synthetic oligomer preps, limited information is available concerning the homogeneity and functional properties of the oligomer molecules which are present in the oligomer preps.

Determining for an Oligomer Prep the Fraction of Oligomer Molecules Which is Capable of Hybridizing with a Complementary Nucleic Acid.

A variety of widely used methods are available for obtaining a measure of the extent of hybridization of complementary nucleic acid molecules which are free in solution, or the extent of hybridization between molecules free in solution and molecules immobilized on a surface. Such methods include a wide variety of spectroscopic methods, a wide variety of enzyme based methods, and a wide variety of methods based on somehow separating hybridized from non-hybridized nucleic acids or oligomers (15,17,21-31).

While all of these methods can be used to determine a measure of the extent of hybridization of an oligomer with a complementary nucleic acid, not all of these methods can be readily used to determine a quantitative value for the fraction of oligomer molecules in an oligomer prep which is capable of hybridizing with a complementary nucleic acid. Herein, the fraction of oligomer molecules in an oligomer prep which is capable of hybridizing with a complementary nucleic acid under a particular hybridization condition, is termed the fraction of oligomer hybridized, or the FH. For many spectroscopic methods determining an accurate FH value is not readily feasible. For example the OM can be used to obtain a measure of the extent of oligomer hybridization, but cannot readily be used to determine an oligomer FH value. A similar situation exists for most standard filter or microarray, hybridization based methods. For these methods the determination of the FH for an oligomer prep is difficult or not feasible. Accurate quantitative values for an oligomer prep FH can readily be obtained with properly designed enzymatic based and non-filter separation based methods. These include nuclease and separation based assays which analyze a hybridization reaction where the oligomer and complementary nucleic acid molecules are free in solution, include the S1 nuclease method, the RNase protection method, hydroxyapatite methods, and size separation methods.

Kinetics of Hybridization of Synthetic Oligomers with Complementary Synthetic Oligomers or Complementary Nucleic Acid Molecules from a Biological Source.

This subject is reviewed extensively in references 15 and 29. Note that for the hybridization of complementary nucleic acid molecules, the terms hybridization, association, re-association, and re-naturation, are interchangeable. Overall, the kinetics of hybridization are influenced by the following factors. (i) Hybridization temperature. (ii) The ionic strength of the hybridization solution. (iii) The pH of the hybridization solution. (iv) Viscosity of hybridization solution. (v) Concentration of each complementary nucleic acid molecule. (vi) Nucleotide length of the complementary nucleic acid molecules. (vii) Nucleotide sequence, nucleotide composition, and secondary structure of the complementary nucleic acids. (viii) The complexity of the complementary nucleic acid molecules. (ix) The degree of complementarity of the hybridizing nucleic acid molecules. (x) The types of complementary nucleic acids hybridizing, that is RNA, DNA, or modified. The general hybridization characteristics of oligomers and biological polynucleotides are similar.

Note that base pair mismatches in the duplex region which are caused by mutation or chemical damage, have essentially no effect on the hybridization kinetics of the damaged, but partially complementary nucleic acids, up to a point where 10% of the duplex region consists of mismatched base pairs (15,31). At about 10% mismatched base pairs the hybridization kinetics are slowed twofold. Because of this, hybridization kinetic analysis is limited in its usefulness for detecting heterogeneity of oligomer molecules in an oligomer prep.

All of the methods for measuring hybridization between oligomers and complementary nucleic acids, which were discussed in the previous section, have been used to determine a measure of the hybridization kinetics of complementary nucleic acids. Not all of these methods are useful for determining the hybridization kinetics of complementary oligomers. For example, the earlier discussed OM does not directly measure the hybridization kinetics for complementary oligomers. The hybridization methods described in the just previous section which allow the accurate determination of the FH value for an oligomer prep are quite suitable for determining accurate hybridization kinetic values for the hybridization of complementary oligomers or the hybridization of an oligomer with a complementary nucleic acid of any kind.

The Nature of Hybridized Oligomer Duplex Molecules.

This discussion will concern exemplary hybridized duplex molecules produced from the hybridization of equimolar quantities of two complementary N=40 synthetic oligomers which were produced as follows. (a) The DNA synthesis was programmed to synthesize two perfectly complementary synthetic oligomer preps. (b) Each oligomer prep was synthesized and completely deprotected and recovered. (c) For each oligomer the N=40 oligomer fraction is isolated, and each purified oligomer prep consists of only N=40 oligomers. (d) Equimolar amounts of each synthetic oligomer N=40 molecule prep were hybridized to completion. (e) At the end of the hybridization step all oligomer molecules were hybridized and associated with an oligomer duplex molecule. (f) All hybridized duplex molecules possessed perfect end to end matching. That is each nucleotide in each duplex is paired with a nucleotide in the other oligomer strand.

In a situation where each oligomer molecule in each duplex oligomer molecule has the intended nucleotide sequence, and intended nucleotide composition, each hybridized duplex molecule which is present in the hybridized duplex population will have the following characteristic. Each nucleotide present in one oligomer strand of the duplex, will be base paired with its intended complementary nucleotide in the other oligomer strand of the duplex. In this situation, each oligomer duplex molecule in the oligomer duplex molecule population is identical to every other oligomer duplex molecule in the oligomer duplex prep. Therefore, the physical-chemical characteristics of each oligomer duplex molecule present in the hybridized oligomer duplex prep are identical to one another, and the hybridized oligomer duplex prep consists of a homogeneous population of oligomer duplex molecules.

It is well known that not all synthesized oligomer molecules have the same synthesized nucleotide length. Because of this, prior art further processes the synthesized oligomer prep in order to enrich for oligomers which have the intended N. Prior art generally represents that such purified oligomers are composed of 90-95% or more of oligomers with the correct N. Prior art generally believes that if a purified oligomer prep consists of essentially all N oligomer molecules, then essentially all of the oligomer molecules present in the purified oligomer prep are identical to one another in their physical chemical characteristics. Further, prior art believes that a preparation of hybridized oligomer duplexes produced from two such purified complementary oligomer preps, consists of hybridized oligomer duplex molecules which are identical to one another. Prior art also believes that for an unpurified synthesized oligomer molecule prep, all N oligomer molecules which are present in the unpurified oligomer prep have identical physical-chemical characteristics.

It is well known that synthetic oligomer and other nucleic acid molecules which are partially complementary can hybridize together to form stable duplexes. The resulting duplex molecules may possess only double strand regions or may possess both double and single strand regions. Further, in the double strand region of a duplex molecule, one or more nucleotides in one strand may not be paired with its intended complement in the other oligomer strand of the duplex.

Determination of the Dissociation Kinetics of Hybridized Oligomer Duplexes.

Prior art quantitative measurements of oligomer and other nucleic acid hybridization kinetics are common. However, prior art quantitative measurements of oligomer duplex dissociation kinetics are much less common and even rare (21-25,27). In addition, the definitive prior art quantitative oligomer duplex dissociation kinetic measurements were done by classical spectroscopic methods around 20-30 years ago (15,21,22,25,32). Such prior art studies concluded the following concerning the general characteristics of the measured quantitative oligomer duplex dissociation kinetics. (a) The kinetics of oligomer duplex dissociation are greatly influenced by temperature, and the oligomer duplex dissociation kinetics are influenced much more by temperature than are the oligomer hybridization kinetics. For one report, at constant ionic strength and over a temperature span of about 14° C., the rate of oligomer duplex dissociation changed by about 18 fold, while the rate of hybridization changed by about 1.5 fold. Another report indicates that at constant ionic strength and over a temperature span of about 14° C., the rate of oligomer duplex dissociation changed by about 30 fold, while the rate of hybridization changed by about 1.5 fold. A third report indicated that at constant ionic strength and over a temperature span of about 20° C., the rate of oligomer dissociation changed by about 147 fold, while the rate of hybridization changed about twofold. These measurements were obtained using spectroscopic methods to analyze short oligomers with Ns from 6 to 8. A fourth report (27) used a filter hybridization method to determine a measure of the dissociation kinetics of perfectly matched N=19 oligomer duplexes and N=19 oligomer duplexes which contained a single mismatched base in the oligomer duplex region. This report indicated that at constant ionic strength and over a temperature span of 20° C., the rate of dissociation of the perfect match duplex changed by about 130 fold, while for a G-T mismatch duplex the rate change was 165 fold, and for an A-A mismatch the rate change was 173 fold. At all temperatures examined (40° C., 50° C., 60° C., in about 1M NaCl, the mismatched oligomer duplexes dissociated about 1.4-3 fold faster than the perfect match duplexes. The oligomer molecule used had an N=19. (b) The kinetics of oligomer duplex dissociation are much less sensitive to changes in ionic strength than are duplex oligomer hybridization kinetics. One report indicates that a constant temperature and over a sodium concentration range of 0.12M to 1M the oligomer duplex dissociation kinetics changes by about twofold at most, while the oligomer hybridization kinetics change by about 30 fold. Another report indicates that over a 0.05M to 1.05M sodium concentration range the oligomer duplex dissociation rates appear to be independent of ionic strength, while the oligomer hybridization kinetics are strongly influenced. (c) As expected, the kinetics of dissociation appear to be first order in form for short RNA and DNA oligomers analyzed by spectroscopic methods, and by the filter method. For the above-described studies and other similar prior art studies, prior art generally tacitly assumes that the oligomer molecule populations analyzed are homogeneous populations of oligomer molecules.

All of the methods for measuring the hybridization kinetics of complementary oligomers or the kinetics of hybridization of oligomers with other complementary nucleic acids which were discussed in an earlier section, can be utilized to obtain a measure of the hybridized oligomer duplex dissociation kinetics. For an oligomer prep, it is clear that the hybridized oligomer dissociation kinetics can be determined only for the fraction of the oligomer prep molecules which are capable of hybridizing with a complementary oligomer or other complementary nucleic acid molecules. In other words, for a particular hybridization and dissociation condition combination, the maximum fraction of the oligomer prep molecules which can be characterized by dissociation kinetics is equal to the FH value for the oligomer prep. Herein, the fraction of the oligomer prep which is present as duplex molecules at time zero of the dissociation kinetic analysis, is termed the fraction of the oligomer prep dissociated value, or the FD value. For a well designed dissociation kinetic analysis, the oligomer prep FH value equals the oligomer prep FD value. For poorly designed and certain other dissociation analysis the oligomer prep FD value may be larger or smaller than the oligomer prep FH value for a particular hybridization condition. The hybridization methods described in an earlier section which allow the accurate determination of the FH value for an oligomer prep are quite suitable for determining the FD and the accurate quantitative dissociation kinetics for hybridized oligomers. In addition, as has been done by the prior art, certain spectroscopic methods associated with a temperature jump method can be used to accurately determine the dissociation kinetics, but not the FD or FH.

### SUMMARY OF THE INVENTION

The present invention has broad application to the practice and improvement of natural and modified RNA and DNA and other oligonucleotide nucleic acid preparations and the use of such oligonucleotides and oligonucleotide preparations in oligomer applications.

The invention facilitated the discovery that most, if not all, chemically synthesized highly purified oligonucleotide preparation contain highly significant levels of physical and functional inhomogeneity. That is, a significant fraction of the oligonucleotide molecules in such preparations are damaged in some manner, and in many if not most cases, such damage causes the oligonucleotide preparation to function less effectively in an intended application.

The invention is based on the discovery that determination of a number of different properties of such oligonucleotide preparations can be performed and are highly useful in characterizing the preparations, and can be used to develop and produce oligomer preparations which have improved functional homogeneity and/or functional characteristics. In addition, the information generated from such determinations and/or the advantages resulting from the use of such improved oligomer preparations allows improved information and results in a wide range of direct oligo applications, as well as indirect applications.

Thus, it is understood that chemically synthesized oligomers are most commonly designed for an intended use or application which involves specific hybridization of the oligomer with an intended complementary nucleic acid target molecule. To accomplish this in an optimal manner, the synthetic oligomer preparation will have characteristics such that all of the oligomer molecules in the prep can form oligomer·target duplexes by specifically hybridizing with an intended complementary target molecule, and all of the oligomer·target duplexes formed have the same duplex stability, and the base paired regions of the oligomer·target duplex molecules have the intended degree of base pair matching. Thus, a functional characterization of an oligomer preparation which has optimal functionality should demonstrate those characteristics. However, conventional oligomer synthesis, purification, and use does not include determination of whether particular oligomer preps have optimal functional characteristics or not.

In order to have the characteristics just indicated above, an optimally functional oligomer preparation must consist of a homogeneous population of oligomer molecules of intended length N, which have the same physical chemical properties. Typically, conventionally produced and purified N oligomer preparations are not homogeneous, and therefore cannot be functionally optimal oligomer preparations.

The present invention provides for evaluation of the functional characteristics of oligomer preparations by determining whether the oligomer preparation is a homogenous population of oligomer molecules of intended length N which all have the same physical chemical properties. Such determinations can include determination of a number of different characteristics of the oligomer of interest as described below. Knowledge of such properties allows the selection of optimal or improved oligomer preparations, development of improved oligomer preparation methods and materials, and improved oligomer applications due to improved results and information from such oligomer applications.

Thus, in a first set of related aspects (aspects 1 and 2), the invention concerns a method for obtaining improved information or results or both concerning the functional homogeneity and/or functional characteristics of the population of the oligomer molecules which are present in a preparation of chemically synthesized or in vitro enzymatically synthesized or biologically synthesized oligomer preparations, involving determining one or more of the following for the oligomer preparation: (a) the FH value for the oligomer prep under analysis conditions of interest; (b) the DK profile for the oligomer prep under analysis conditions of interest; (c) the % SF and % FF values for the oligomer prep under analysis conditions of interest; (d) the SF and FF t.5d values for the oligomer prep under analysis conditions of interest; (e) the pattern of nucleotide sequence damage associated with the oligomer preparation's FF; and (f) the extent of nucleotide sequence damage associated with the oligomer preparation's FF.

In this and other aspects and embodiments concerning determination and/or use of the FH value for the oligomer prep under analysis conditions of interest; the DK profile for the oligomer prep under analysis conditions of interest; the % SF and % FF values for the oligomer prep under analysis conditions of interest; the SF and FF t.5d values for the oligomer prep under analysis conditions of interest; the pattern of nucleotide sequence damage associated with the oligomer preparation's FF; the extent of nucleotide sequence damage associated with the oligomer preparation's FF, in particular embodiments; the hybridization kinetic association constant kₐ value; particular embodiments involve the determination or use or 1, or of each combination of 2, 3, 4, 5, or 6 of those results or information, or of all 7.

In some embodiments, the method can alternatively or in addition involve determining the hybridization kinetic association constant kₐ value or values for the total oligomer prep under one or more analysis conditions of interest, such as one or more reference conditions and/or one or more conditions of use, intended use, or potential use.

Similarly, a related aspect (aspect 3) concerns a method for obtaining improved information and results concerning whether the measured functional homogeneity and functional characteristic values of a chemically synthesized crude or purified oligomer prep are equivalent to the measured intended functional homogeneity and functional characteristic values of the oligomer prep, where the method includes comparing one or more functional homogeneity/or and functional characteristic values for the chemically synthesized version of the oligomer prep and the biologically or in vitro enzymatically synthesized version or both of the oligomer prep. Such functional homogeneity and/or functional characteristic values can include one or more of (a) the FH values for each total oligomer prep under analysis conditions of interest; (b) the DK profile for each total oligomer prep under analysis conditions of interest; (c) the % SF and % FF values for each total oligomer prep; (d) the SF and FF t.5d values for each oligomer prep under analysis conditions of interest; (e) the pattern of nucleotide sequence damage associated with each oligomer preparation's FF; and (f) the extent of nucleotide sequence damage associated with each oligomer preparation's FF. Such comparisons can be used, for example, in methods to identify sources of damage or heterogeneity in oligomer preps, to determine whether purification (e.g., particular types of purification) are needed for an oligomer prep, and/or to determine whether a particular oligomer prep satisfies particular functional requirements (e.g., by have functional homogeneity and/or functional characteristic values sufficiently close to the intended or reference oligomer prep).

In particular embodiments, the method includes determining one or more of such functional homogeneity and functional characteristic values for one or more of a chemically synthesized oligomer prep, an in vitro enzymatically synthesized version of that oligomer prep, and a biologically synthesized version of that oligomer prep.

Also, as with the preceding aspects, in certain embodiments, the method also or alternatively involves determining and comparing the hybridization kinetic association constant kₐ value or values for the total oligomer preps under the analysis conditions of interest.

The invention likewise, in another related aspect (aspect 4), provides a method for obtaining improved information and results concerning the degree of effectiveness of a chemically synthesized or in vitro enzymatically synthesized or biologically synthesized oligomer for an intended oligomer application, where the method involves determining one or more (e.g., a plurality) of the oligomer prep functional characteristic values for FH, % SF and % FF, SF t.5d, FF t.5d, pattern of FF nucleotide sequence damage, extent of FF nucleotide sequence damage, and hybridization kinetic kₐ under one or more application conditions of interest, for one or more oligomer preps which are designed and produced for an oligomer application, and determining whether the oligomer prep functional characteristic values satisfy the functional effectiveness requirements for an oligomer prep in the oligomer application of interest.

In particular embodiments, the intended oligomer application is or includes one of more of the following: (i) an RT-PCR or PCR application; (ii) an enzymatic synthesis of RNA or DNA application; (iii) a nucleic acid synthesis primer application; (iv) a nucleic acid sequencing application; (v) a gene cloning application; (vi) a gene expression analysis or gene expression comparison analysis application, such as a Serial Analysis of Gene Expression (SAGE) or other clone counting analysis; (vii) a nucleic acid hybridization application; (viii) a DNA and/or RNA mutation detection or SNP detection application; (ix) a method of calorimetry analysis for determining oligomer physical-chemical and thermodynamic information application; (x) a determination of oligomer Tm values by OM analysis application; (xi) a determination of oligomer duplex equilibrium constants by OM or other methods analysis application; (xii) the use of an oligomer as a standard for an oligomer application or other application; (xiii) a nucleic acid ligation application; (xiv) a fluorescent labeled oligomer application; (xv) a radioactive labeled oligomer application; (xvi) a chemiluminescent labeled oligomer application; (xvii) an enzyme labeled oligomer application; (xviii) a metal or non-metal nano particle label oligomer application; (xix) a hybridized duplex strand displacement application; (xx) a biotin labeled oligomer application; (xxi) a ligand molecule labeled oligomer application; (xxii) a receptor or binding molecule application; (xxiii) an application using a chemically modified oligomer; (xxiv) a use of modified or unmodified oligomers to form nano-structures or patterns or functions application; (xxv) a DNA or RNA oligomer application; (xxvi) an antisense or aptamer DNA or RNA application; (xxvii) a pharmaceutical antibiotic, anti-viral, or therapeutic or vaccine application; (xxviii) a gene synthesis application; (xxix) a regulatory siRNA, miRNA, or RNA or DNA gene expression suppression or enhancement application; (xxx) a site directed mutagenesis application; and/or (xxxi) a discovery and identification of expressed particular gene(s) and gene expression profile(s) which are characteristic of one of more particular normal state(s) or disease state(s) application. The preceding list may be referred to herein as the “application list”.

In yet another related aspect (aspect 5), the invention further provides a method for improving oligomer application results, where the method involves improving the functional effectiveness of an oligomer application utilizing an oligomer prep by: (a) determining the oligomer prep functional characteristic values for one or more (e.g., a plurality) of FH, % SF and % FF, SF t.5d, FF t.5d, pattern of FF nucleotide sequence damage, extent of FF nucleotide sequence damage, and hybridization kinetic kₐ under one or more application conditions of interest, for one or more oligomer preps which are designed and produced for an oligomer application; (b) quantitating or otherwise determining the functional effectiveness of each oligomer prep in the oligomer application of interest; (c) quantitating or otherwise determining whether the actual functional effectiveness of the application is the intended functional or desired effectiveness; (d) correlating the functional characteristic values for each oligomer prep with the oligomer prep's functional effectiveness value, in order to identify an improved useful or optimal set of oligomer functional characteristics for the intended oligomer application; and (e) using the identified oligomer prep for the intended application to obtain improved oligomer application results.

In particular embodiments, the oligomer application or intended oligomer application is or includes one of more of applications listed in the preceding aspect.

Also, as with the aspects above, in particular embodiments of the above aspects, the analysis condition of interest is a reference analysis condition and/or a condition of interest specific for a particular oligomer application.

Another related aspect (aspect 6) relates to a method for improving functional effectiveness of oligomer preps prepared using a particular synthesis method by (a) determining at least one of the oligomer prep functional characteristic values FH, % SF and % FF, SF t.5d, FF t.5d, pattern of FF nucleotide sequence damage, extent of FF nucleotide sequence damage, and hybridization kinetic kₐ under one or more conditions, for an oligomer prep produced using an original synthesis method; (b) determining the at least one oligomer prep functional characteristic value under those conditions for at least one experimental oligomer prep produced using a modified synthesis method in which one or more parameters of the synthesis method is varied; and (c) selecting the modified synthesis method as an improved synthesis method if the functional characteristic values for the modified synthesis method are improved over the functional characteristic values for the original synthesis method. The method can include a number of different synthesis conditions (e.g., 2-5,6-10, 11-20 different conditions, or more. Likewise, the method can provide an optimization process in which parameters are varied iteratively, with sequential selection of conditions which identify oligomer preps with progressively improved functional characteristic values, e.g., 2, 3, 4, 5, 6, 7-10, or more rounds of varying, testing, and selecting.

As with above aspect, in particular embodiments the analysis condition of interest is a reference analysis condition and/or a condition of interest specific for a particular oligomer application.

Further, in particular embodiments, the homogeneity is functional homogeneity in an intended oligomer application; the synthesis method is carried out in an automated synthesizer; the synthesis method includes an enzymatic DNA synthesis method or an enzymatic RNA synthesis method;

Another aspect (aspect 7) of the invention concerns a method for improving functional effectiveness of oligomer preps prepared using a particular synthesis method, by (a) determining at least one of the oligomer prep functional characteristic values FH, % SF and % FF, SF t.5d, FF t.5d, pattern of FF nucleotide sequence damage, extent of FF nucleotide sequence damage, and hybridization kinetic kₐ under one or more conditions, for a plurality of oligomer preps selected for potential use in an oligomer application; and (b) selecting an oligomer prep from the plurality of oligomer preps having better functional characteristic values for the oligomer application.

Once again, as in above aspects, in some embodiments, the analysis condition of interest is a reference analysis condition and/or the condition of interest specific for a particular oligomer application.

Also in particular embodiments and aspects, the functional effectiveness corresponds to functional homogeneity of said oligomer prep; the functional effectiveness corresponds to a functional characteristic of said oligomer prep; the oligomer application is an application as listed for the aspect two above; a plurality of the oligomer preps are synthesized using a plurality of different synthesis methods; the plurality of different synthesis methods is or includes a plurality of modifications of one synthesis method; for the plurality of modifications of one synthesis method, one or more of the functional characteristics of the oligomer preps produced from the plurality of modifications of one synthesis method are compared to select an optimized synthesis method producing improved oligomer preps for the oligomer application.

A further aspect (aspect 8) concerns a method for converting quantitative functional homogeneity and functional characteristic values for an oligomer preparation obtained under one analysis condition, to quantitative functional homogeneity and functional characteristic values which are correct for that same oligomer preparation under different analysis conditions. The method involves determining the quantitative functional homogeneity and functional characteristic values for an oligomer preparation under one analysis condition chosen as a reference condition; determining the quantitative functional homogeneity and functional characteristic values for the same oligomer preparation under a different analysis condition; and determining a quantitative conversion factor for correctly converting each quantitative functional homogeneity and functional characteristic value obtained under one condition, to a quantitative functional homogeneity and functional characteristic value which is correct for the other analysis condition. Those of skill in the field are familiar with determining such conversion factors.

For any of the above aspects and embodiments, in particular embodiments the oligomer preparation molecules of interest not attached to a solid phase substrate (e.g., are free in solution); the oligomer preparation molecules of interest are immobilized on a surface (e.g., any of the solid phase media mentioned herein for immobilization of oligomers).

The ability to determine and use information and results on the functional homogeneity and/or functional characteristic values provides, in another aspect (aspect 9), a method for obtaining improved information and results for a further oligomer application which utilizes improved oligomer application information or results. The method involves using any of the methods described herein for obtaining improved information or results (e.g., as in aspects above) in an oligomer application, and utilizing the improved oligomer application information and results in the further application of interest to obtain improved information for the further oligomer application of interest which utilizes the improved oligomer application information and results.

In particular embodiments, the further application of interest is a product or service associated with: biological; human medical, agricultural; veterinary; nutrition; forensic; public health; ecological; bio-warfare; toxicology; diagnostic assays; basic, industrial and applied research or development; the application is or involves an application in the application list above; the application of interest is, includes, or is an aspect of one or more of the discovery of pharmaceutical drugs or bioactive compounds, the evaluation of the specificity, toxicity, or efficacy or any combination thereof of pharmaceutical drugs or bioactive compounds, the development of drug or bioactive compound related diagnostic assays, the improvement or optimization of a drug or bioactive compound's specificity, toxicity, efficacy, or pharmacokinetic characteristics or any combination thereof, the identification of drug or bioactive compound clinical screening participants or the drug or bioactive compound's market niche or both, the quality control and assurance for drug and bioactive compound drug production, and the efficient prescription and use of the drug or bioactive compounds (e.g., employing or contributing to a pharmacogenomics application, or an individualized medicine application involving determination of particular genetic characteristics of an individual).

The ability to use the present methods to detect and quantitate oligomer damage in oligomer preparations provides, in another aspect (aspect 10), a method for obtaining improved information and results concerning the contribution to detectable oligomer molecule damage of a putative oligomer damage factor by using the described functional heterogeneity and functional characteristic values to guide identification of the various damage factors and their contribution. Thus, the method can involve establishing an experimental design for validly testing the effect of the factor of interest, or differences in the factor of interest, on the extent of oligomer damage; producing a plurality of oligomer preps for carrying out the experimental design; determining for each of the oligomer preps a quantitative measure of the functional homogeneity of the oligomer prep, and quantitative values for one or more of the oligomer prep functional characteristics FH, % SF, % FF, SF t.5d, FF t.5d, the pattern of nucleotide sequence damage, and the extent nucleotide sequence damage associated with the oligomer and oligomer FF, and if necessary the oligomer prep kₐ; analyzing the measured values for the functional homogeneity and functional characteristics of the oligomers tested; and determining a measure of the quantitative effect of the putative oligomer damage factor on detectable oligomer damage to identify any actual damage factor.

In particular embodiments, the putative damage factor(s) includes one or more of: a synthesis reagent factor, a synthesis process factor, a synthesis protocol factor, a synthesis instrument factor, a synthesized oligomer processing factor, a synthesized oligomer purification factor, a synthesized oligomer fractionation factor, a synthesized oligomer concentration factor, a synthesized oligomer characterization factor, a synthesized oligomer storage factor, a synthesized oligomer application factor.

A related aspect (aspect 11) concerns a method for reducing or eliminating the oligomer damage associated with one or more actual damage factors in an oligomer preparation process. The method involves varying one or more identified actual damage factors (e.g., as identified in the preceding aspect) to create test damage factors; producing and testing oligomer preparations using said one or more of the test damage factors to identify test damage factors which result in reducing or eliminating the detectable oligomer damage associated with the actual damage factors, thereby identifying improvements in the oligomer preparation process. One or more of those identified improvements can be utilized in the oligomer preparation process to produce less damaged oligomer preparations.

In particular embodiments, the actual damage factor is identified using the method of the preceding aspect; the method of the preceding aspect is used to identify improvements for each actual damage factor.

In addition to providing for the improvement of methods and compositions for preparing oligomer preparations, further aspects concern producing improved results and/or information in oligomer applications, either directly or indirectly. One such aspect (aspect 12) concerns a method for producing improved information and results for a zero order application (a zero order oligomer application) which directly utilizes measured oligomer and oligomer preparation functional homogeneity and functional characteristic results, where the method involves using the methods described herein for producing or determining improved oligomer and oligomer preparation functional homogeneity and functional characteristic information and results (e.g., any of claims 1-13); and utilizing a part or all of those improved oligomer and oligomer preparation functional homogeneity information and results in a zero order application such that one or more improved zero order application information and result is produced.

In particular embodiments, the zero order application is or includes one or more of a method for producing DNA, RNA, or modified RNA or DNA synthesis reagents; a method for producing oligomer duplex of any kind equilibrium constants; a method for producing oligomer primers of all kinds for the in vitro enzymatic synthesis of RNA or DNA; a method for producing oligomers for use in a nucleic acid ligation process; a method for producing oligomer capture probes for gene expression analyses; a method for producing oligomer RNA or DNA diagnostic probes; a method for producing SNP and base pair mismatch detection oligomers; a method for producing site directed mutagenesis oligomers; a method for producing oligomers for use in gene synthesis; a method for producing oligomer siRNAs and miRNAs and other regulatory RNAs; a method for producing molecular beacon, FRET, and other fluorescent molecule associated oligomers; a method for producing oligomers for use in nucleic acid sequencing; a method for producing improved oligomer assay and other standard; and a method for producing synthesis processes, protocols, and QA/QC procedures.

In similar manner, in another aspect (aspect 13) the invention provides a method for producing improved information and results for a first order application which directly utilizes zero order application results, where the method involves using a method as described herein to obtain improved zero order application information and results (e.g., a method of any of claims 1-13 and 37-38), and utilizing those improved zero order application results and/or information in a first order application, producing one or more improved first order application information and results.

In particular embodiments, the first order application is or includes one or more of one or more methods for producing lot to lot reproducibility for chemically synthesized or enzymatically synthesized oligomers or both; one or more methods for the manufacturing of chemically synthesized or enzymatically synthesized or both oligomer preparations; one or more methods for producing oligomer and oligomer duplex thermodynamic property results; one or more methods for producing primer dependent RNA and DNA enzymatic synthesis results; one or more methods for producing gene expression analysis and gene expression comparison microarray, RT-PCR, and other, assay results; one or more methods for producing results for a oligomer associated nucleic acid ligation assay or procedure; one or more methods for producing results for an oligomer associated biological assay; one or more methods for producing SNP and mismatched base pair detection assay results; one or more methods for producing site directed mutagenesis procedure results; one or more methods for producing oligomer based gene synthesis procedure results; one or more methods for producing siRNA or miRNA or other oligomer regulatory RNA assay results; one or more methods for producing results for assays which utilize molecular beacons, FRET, and other fluorescent molecule associated oligomers; one or more methods for producing results for assays which utilize RNA or DNA oligomer standards; one or more methods for producing results for nucleic acid sequencing assays and methods which utilize oligomers; one or more methods for producing results for oligomer associated PCR assays and methods; and one or more methods for producing results for oligomer associated SAGE and other clone counting gene expression assay methods.

Another aspect (aspect 14) similar to the above concerns a method for producing improved information and results for a second order application which directly utilizes first order application results (and may also utilize other order application results), where the method involves using a method as described herein to produce improved first order application information and results (e.g., a method of any one or more of claims 1-13 and 39-40); and utilizing all or part of those improved first order application information and results in a second order application to produce one or more improved second order application information and results.

In particular embodiments, the second order application is or includes one or more of one or more methods for producing procedures for accurately predicting the physical, chemical, or functional characteristics of oligomers and oligomer duplexes or oligomer-target duplexes in an oligomer application; one or more methods for producing results for applications which utilize improved oligomer primer dependent enzymatic synthesis or ligation methods; one or more methods for producing data mining analysis results; and one or more methods for producing drug or bioactive molecule or biomarker or other product candidate discovery, identification and validation results.

In a further related aspect (aspect 15), the invention provides a method for producing improved information and results for a third order application which directly utilizes second order application results by using a method of the invention (e.g., the method of any one or more of the claims 1-13 and 41-42) to produce improved second order application information and results; and utilizing all or part of those improved second order application information and results in a third order application, producing one or more improved third order application results.

In similar manner to aspects above, in particular embodiments the third order application includes one or more of one or more methods for producing improved oligomer associated assays; one or more methods for producing improved systems biology analysis; and one or more methods for producing an improved drug or bioactive molecule or biomarker or other product candidate screening and selection results and processes.

Once again, in a further related aspect (aspect 16), the invention concerns a method for producing improved information and results for a higher order application, which directly utilizes one or more lower application results, by using a method as described herein (e.g., any of one or more of claims 1-13 and 37-44) to produce improved lower order application information and results; and utilizing all or part of the improved lower order information and results in a higher order application, thereby producing one or more improved higher order application results.

In accordance with aspects above, in particular embodiments the lower order application is or includes one or more of the following: a zero order application; a first order application; a second order application; a third order application; and a higher than third order application.

In certain embodiments, the higher order application is or includes one or more of: one or more methods for producing drug or bioactive molecule or biomarker clinical study candidate selection results; one or more methods for producing drug or bioactive molecule or biomarker clinical study evaluation results; one or more methods for producing drug or bioactive molecule or biomarker manufacturing and QC/QA results; one or more methods for producing drug or bioactive molecule or biomarker or other product market segment selection process results; one or more methods for producing drug or bioactive molecule or biomarker or other product prescription and use in the patient results; one or more methods for producing drug or bioactive molecule or biomarker efficacy in the patient; one or more methods for producing drug or bioactive molecule or biomarker or other product toxicological characteristic results; one or more methods for producing disease or pathology state prognosis prediction results; and one or more methods for producing disease or pathology state prognosis prediction after drug or bioactive molecule or other product treatment.

Another aspect (aspect 17) of the invention concerns a characterized oligomer preparation which includes an oligomer preparation and a data set embedded in a hardcopy, computer display, or electronic data storage medium describing one or more characteristics of the oligomer preparation. At least some of the data set is data representing improved oligomer functional homogeneity information or results or both and/or improved functional characteristic information or results or both. Such information may be produced using the methods described herein for obtaining such information and results, for example, one or more methods as described above.

In particular embodiments, the characterized oligomer is a surface immobilized oligomer (for example, immobilized on any of the solid phase media mentioned herein); the characterized oligomer is not surface immobilized (e.g., the oligomer is free in solution); the information and results includes at least one of functional characteristic values for FH, % SF and % FF, SF t.5d, FF t.5d, pattern of FF nucleotide sequence damage, extent of FF nucleotide sequence damage, and hybridization kinetic kₐ corresponding to said oligomer preparation under one or more conditions of interest.

In view of the capability to use the present invention to produce or select improved oligomer preparations, the invention also concerns a kit which includes at least one packaged combination (e.g., in a box(es), vial(s), or shrink-wrapped assemblage of containers) of at least one oligomer preparation and a data set embedded in a hardcopy, computer display, or electronic data storage corresponding to the oligomer preparation and containing information or results or both relating to oligomer functional homogeneity or functional characteristics or both. Alternatively, the kit may include a packaged oligomer preparation(s), in some cases the data concerning the oligomer preparation is accessible separately, e.g., via the internet.

In particular embodiments, the information or results or both includes one or more oligomer prep functional characteristic values for FH, % SF and % FF, SF t.5d, FF t.5d, pattern of FF nucleotide sequence damage, extent of FF nucleotide sequence damage, and hybridization kinetic kₐ under one or more conditions of interest; the conditions of interest are or include application conditions; the conditions of interest are or include reference conditions; the kit also includes instructions for use of said oligomer prep; the kit also includes a buffer; the kit also includes separate quantities of components for synthesis of an oligomer (e.g., in separate chambers, vials, or other containers); the kit is an enzymatic DNA or RNA synthesis or ligation kit (e.g., a PCR, RT-PCR, or LCR kit); the kit is a hybridization probe kit.

Another aspect (aspect 18) of the invention concerns a data set at least partially describing characteristics of at least one oligomer prep, where the data set specifies one or more characteristics of the oligomer preparation and includes data corresponding to at least one of oligomer functional homogeneity, and oligomer functional characteristics (e.g., as values and/or type and pattern of damage). Generally the data set is embedded in a physical medium such as in a hard copy (e.g., in a database contents printout, product insert, or product spec sheet), computer display, or electronic data storage medium.

In particular embodiments, the data set includes data for oligomers complementary to oligomers in the oligomer preparation; the data set is for one oligomer preparation; the data set is for at least 2, 5, 10, 100, 1000, or more oligomer preps; the data set includes data at least partially describing improved functional homogeneity and improved functional characteristic values obtained under one or more known conditions of solution composition, pH, temperature, pressure, and electric field strength, for each of a plurality of different oligomers which differ in nucleotide sequence and/or nucleotide length and/or nucleotide composition; the data set includes data describing at least one of FH, % SF and % FF, SF t.5d, FF t.5d, pattern of FF nucleotide sequence damage, extent of FF nucleotide sequence damage, and hybridization kinetic kₐ for said oligomer preparation; oligomers in the oligomer prep or complement thereto include modified nucleotides; each different oligomer is hybridized to one or more different complementary nucleic acid molecules which may different in length, nucleotide sequence, and/or content (e.g., inclusion and/or location of nucleotide analogs or nucleotide substitutes).

In certain embodiments, some or all of the data in the data set is for surface immobilized oligomers or for oligomers which are not surface immobilized (e.g., free in solution); the improved functional homogeneity and functional characteristic values are determined for surface immobilized or for non-surface immobilized oligomers.

Also in particular embodiments, the data set includes one or more improved thermodynamic (TD) property values (e.g., improved using the present methods (such as those for determining functional homogeneity and functional characteristic values), e.g., TD values which are pertinent for designing oligomers for particular oligomer applications; improved thermodynamic values are determined using invention improved information or results or both for at least one said oligomer preparation; the electronic data storage medium is computer memory (e.g., RAM and/or ROM memory), a portable computer accessible data storage device (e.g., CD, DVD, optical disk, flash memory device, and the like).

A further aspect (aspect 19) of the invention provides a method for predicting the functional characteristic t.5d and ka values under specified hybridization and duplex dissociation solution composition, pH, temperature, pressure, and electric field strength conditions, for an oligomer which has a particular nucleotide sequence, nucleotide length, and nucleotide composition, and an oligomer complementary nucleic acid which has a particular nucleotide sequence, nucleotide length, and nucleotide composition, by applying rules (e.g., a rule set) for predicting the t.5d and ka values for the oligomer under said conditions to determine the t.5d and ka values.

Typically those rules are established based on a set of data for a large plurality of oligomer preparations, where the data set includes at least one of FH, % SF and % FF, SF t.5d, FF t.5d, pattern of FF nucleotide sequence damage, extent of FF nucleotide sequence damage, and hybridization kinetic kₐ for each of the large plurality of oligomer preparations, and can further be based in part on the functional characteristic FH values for the oligomers and their complementary sequences.

In particular embodiments, the method for determining rule basis data set are as described for aspects and embodiments above and/or derived using normal relationships, e.g., equilibrium, kinetic, and/or thermodynamic relationships; the rule(s) include analytic algorithms and/or look-up tables; the oligomer and/or its complement are modified, such as by the inclusion of nucleotide analogs, nucleotide substitutes, and/or intended damage.

In view of the ability and knowledge to obtain and/or provide functional characteristic values, as well as kinetic and/or thermodynamic values derived therefrom, provides an aspect (aspect 20) concerning a method for selecting one or more particular oligomers for use in an oligomer application, where the method includes identifying a particular oligomer based on having at least one of improved functional homogeneity, functional characteristics, or improved thermodynamic property values (which may be improved in accordance with aspects described above or otherwise described herein) appropriate for an intended oligomer application. In particular embodiments, the functional homogeneity, functional characteristics, or improved thermodynamic property values are determined according to any of the aspects and embodiments described above for determining such properties and values.

In particular embodiments, the method involves determining application conditions which are compatible with the particular application; identifying at least one oligomer having or expected to have desired functional homogeneity and functional characteristics under those application conditions. The method can also include selecting a set of oligomer nucleotide sequences, nucleotide lengths, and nucleotide compositions which may be effective for the application (e.g., test oligomers); and designing at least one oligomer utilizing at least one data set as described above, where the oligomer is expected to have the desired functional homogeneity and/or functional characteristics under the application conditions, thus providing a designed oligomer. The method can also include producing one or more of such designed oligomers and determining for each oligomer the functional homogeneity and functional characteristic values for the oligomer under one or more application conditions; and testing the effectiveness of each oligomer and its associated condition in the application of interest. Still further, the method can include evaluating the effectiveness of each oligomer in the application while taking into account the measured functional homogeneity and functional characteristic values of each oligomer. The application can, in addition, include performing the process for alternate oligomer candidates (e.g., at least 2, 5, 10, 20 or more such alternate oligomer candidates), and further can include comparing the results of the evaluation for each oligomer candidate; and selecting one or more oligomer candidates for use in the oligomer application based on such comparison. The selection can, for example, be based on the oligomer having desirable effectiveness in the application and/or having desirable functional homogeneity or functional characteristic values.

A related aspect (aspect 21) relating to oligomer selection and/or design for oligomer applications concerns a method for in silico design and/or selection of oligomers for an application. The method involves the use of a computer interface (e.g., for a general purpose computer) and linked computer software for performing the design and/or selection, and involves identifying the oligomer application or set of applications (e.g., related applications and/or applications having related conditions), accessing a database containing t.5d, kₐ, and thermodynamic values (TD) for a large plurality (e.g., at least 10, 25, 50, 100, 200, 400, 600, 800, 1000, or even more) of different oligomers, or large plurality of different application conditions, or large plurality of different combinations of different oligomers and different application conditions, and/or accessing one or more electronically programmed algorithms based on thermodynamic values, and oligomer characteristic results or information or both which predicts t.5d, kₐ, and thermodynamic values for an oligomer under particular conditions (e.g., data as in a data set as described above), identifying at least one oligomer having t.5d, kₐ, and thermodynamic values suitable for that oligomer application using the linked software.

In particular embodiments, identifying the oligomer application includes specification of application conditions (e.g., at least one condition from hybridization conditions, wash conditions, polymerization conditions, and dissociation conditions); identifying the oligomer application includes specification of oligomer requirements, for example, at least one requirement from nucleotide sequence, intended target sequence, stability of oligomer-target sequence duplex in application or portion thereof; stability of oligomer-non-target sequence stability in application or portion thereof; difference in duplex stability between oligomer-target sequence duplexes and oligomer-non-target duplexes; intended state of oligomer-target duplexes in application.

In particular embodiments, the oligomer application includes use of an oligomer which is a primer, a hybridization probe, a capture probe, or a ligation oligonucleotide; an intended oligomer sequence is specified or identified, and the method can also include evaluating potential duplexes between the oligomer and non-target sequences in the application; the method includes determining t.5d, kₐ, and TD values for the oligomer (e.g., using a program with functions for performing such calculations); the method includes identification of matches between oligomer application requirements and predicted application properties for one or more particular intended oligomers, and can also predict effects on application results due to one or more patterns or levels or both of damage in a particular oligomer.

In keeping with the data sets and related methods, further aspects include computer programs for carrying kinetic and/or TD value calculations, selection of oligomer(s) for particular applications, and/or for determining the effects of oligomer damage in an oligomer application.

Thus, one such further aspect (aspect 22) concerns a computer program for calculating t.5d, kₐ, or thermodynamic values or any combination thereof for a particular oligomer. Such program includes a set of computer instructions embedded in a computer accessible storage medium, where the instructions operate on input data which includes functional characteristic values for an oligomer preparation and return t.5d, kₐ, and thermodynamic values for at least one particular oligomer preparation.

Another such aspect (aspect 23) concerns a computer program for selecting a particular oligomer for a particular oligomer application, where the program includes a set of computer instructions embedded in a computer accessible storage medium, where the instructions calculate predicted performance of one or more particular oligomers in a particular oligomer application under specified conditions, and identify one or more oligomers having better predicted performance in that application.

Likewise, another such aspect (aspect 24) concerns a computer program for determining the effects of oligomer damage in an oligomer application, such that the program includes a set of computer instructions embedded in a computer accessible storage medium, where the instructions calculate predicted application performance for one or more oligomer preparations having particular types and/or levels of nucleotide sequence damage. The instructions can further identify oligomer preparations which have better performance or which have a performance which satisfies a particular level in the application.

The aspect and embodiments above have been described with reference to oligomers. In addition, in related aspects, the invention includes application of those oligomer aspects to other dissociable specific binding complexes, such as bi-molecular complexes. Examples include protein·protein, protein·hapten, antibody·antigen, and antibody·hapten binding, as well as protein·nucleic acid complexes. It is recognized that such molecules are subject to heterogeneity (e.g., reflecting sequence or other structural damage) which alters performance of the molecules in particular applications, so that essentially the same determinations and methods can be carried out as for the oligomers.

The phrase “analysis conditions of interest” refers to physical/chemical conditions under which a particular analysis is carried out or for which the analysis results are adjusted. Similarly, the phrase “application conditions” or “application conditions of interest” refers to physical/chemical conditions intended, expected, or experienced during the conduct of a particular application. Examples of properties defining such conditions can include temperature, pressure, solution chemical properties, electric field, and the like. Similarly, a “reference condition” is a condition of interest which has been selected and/or accepted for comparison purposes, for example, because that condition or conditions is relatively easy to consistently reproduce, is relatively inexpensive to produce, reasonably represents a range of application and/or analysis conditions, has previously been used for similar or related applications and/or analyses, and other such factors.

In the context of an oligomer, the terms “chain moiety” and “backbone moiety” interchangeably refer to a portion of the oligomer which results from incorporation of a molecular species in the chain. Unless clearly indicated to the contrary, such chain moieties may include nucleotides, nucleotide analogs, and nucleotide substitutes.

As used in connection with oligomer preparations, the terms “chemically synthesizing” refers to the process of covalently linking moieties including nucleotides and/or nucleotide analogs in the oligomer chain, but not using enzymes in that process.

In the context of the separation of an oligomer chain into two or more chain portions, the term “cleaving” refers to the breaking of one or more bonds which form part of the backbone of the chain creating two or more separate chain portions. Thus, “specifically cleaving” refers to cleavage which occurs predominantly (and preferably exclusively or nearly exclusively) at a particular defined site or sites in the oligomer chain. Such cleavage may, for example, be performed enzymatically (e.g., using a nuclease) or non-enzymatically (e.g., using non-enzymatic chemical conditions).

The extent of nucleotide sequence damage associated with the first synthesized end of a CS oligomer molecule is termed the “Damaged nucleotide Site Density”, or DSD for an oligomer prep. The DSD reflects the average number of damaged nucleotide sites per nucleotide for an oligomer molecule prep. For reference 9: the DSD value for the first five nucleotides added to a growing oligomer nucleotide chain is about four times greater than the DSD for the second five nucleotides added to the same growing chain; and the DSD value for the first ten nucleotides added to a growing oligomer chain is about three to four times greater than the DSD for the second ten nucleotides added to the same growing oligomer chain. Such sequence damage includes insertions, deletions, and damage to the structure of a nucleotide.

In connection with such damage, the phrase, “extent of nucleotide sequence damage” (also referred to simply as extent or level of damage) and like terms reflect the number of chemical/physical modifications producing nucleotide sequence damage along the oligomer. Similarly, the phrase “pattern of nucleotide sequence damage” means the placement of nucleotide sequence damage along the oligomer, and can include distinction of different types of damage.

The term “data set” means a compilation or assemblage of data, e.g., oligomer data relating to functional homogeneity, functional characteristics and characteristic values, kinetic parameters, equilibrium parameters, and/or thermodynamic parameters. Such data set may include data for one or more oligomers or oligomer preparations, and may also include additional data.

In specifying nucleotide sites in an oligomer, reference to the “first nucleotide” or “first nucleotide position” or site, or first 10 nucleotides positions or sites is defined with reference to the synthesis direction from the particular reference point (e.g., the first nucleotide position in a wanted polymer), such that the first nucleotide incorporated in the growing chain is the first nucleotide, and so on for the specified number of nucleotides. Thus, the counting may proceed in the 5′ to 3′ direction or 3′-5′ direction depending on the direction of synthesis.

When used in connection with the use of an oligomer or oligomer preparation in an application, the term “functional effectiveness requirements” refers to function-related requirements for use of oligomers in an oligomer application, and to indicators of how well the oligomer or oligomer preparation functions in that application.

As applied to an oligomer preparation, the term “functional homogeneity” refers to the extent to which the oligomers in an oligomer preparation possess the same length, sequence, and other physical/chemical properties which affect the function of the oligomers in an oligomer application.

“Functional characteristics” and “Functional characteristic values” refer to functionally relevant parameters or properties of an oligomer or oligomer prep reflecting its physical behavior (e.g., under particular application conditions), and values describing that behavior. Particular examples of such functional characteristic values are described herein.

As used herein the term “immobilized oligonucleotide” and like term such as immobilized polymer, immobilized chain, and immobilized oligomer refer to such chains which are attached to a solid phase medium in such manner that the attachment is stable under the relevant conditions. In many cases, the attachment is a covalent bond linkage.

Use of the term “improved” herein in the context of properties, characteristics, results, information, values, and the like indicates that the indicated corresponding items are better in some recognizable sense, especially better accuracy and/or precision, and/or the provision of previously unavailable properties, characteristics, results, information, values, and the like.

The term “intended nucleotide length”, intended oligomer length”, “intended chain length” and like terms refer to the design length for a particular chain. It is the length which all chains would have in a perfect synthesis such that all the synthesized oligomers have the same, design length.

Similarly, the terms “intended functional homogeneity” and “intended functional characteristic values” refer to the homogeneity and functional characteristic values for a design oligomer preparation, that is, a preparation in which all the oligomers have the same, design nucleotide sequence and designed nucleotide length, and are free of nucleotide sequence damage. Thus, the oligomers will all have the same physical/chemical properties. Such intended oligomer preparation and associated properties can be approximated by a high quality enzymatically synthesized oligomer preparation.

In the present context, the phase “measured intended functional homogeneity and functional characteristic values of the oligomer prep” refers to the measured functional homogeneity and functional characteristic values respectively of a high quality reference oligomer preparation (e.g., a gold standard reference, which may be approximated by a purified enzymatically synthesized oligomer preparation), because such a preparation will have functional homogeneity and functional characteristic values approximating those for the intended oligomer, that is, the design standard oligomer. High quality reference preparations may be provided by good quality (e.g., highly purified) enzymatically synthesized oligomer preparations. For example, the purified oligomers may be prepared using enzymatic synthesis with a high fidelity polymerase enzyme on a template which codes for restriction sites at the appropriate oligo boundaries, and the resulting free oligos purified under conditions which do not damage the oligos. Such high quality enzymatically synthesized oligomer preparations may be used for such reference preparations because of the high fidelity they provide. Advantageously, higher fidelity polymerases are used for synthesizing such reference preparations. Additionally, the synthesis can be carried out in the presence of an enzymatic error correction apparatus to further reduce the error rate.

Herein, the terms “nucleoside” and “nucleotide” refers to one or more of the naturally occurring nucleosides or nucleotides, as for example the naturally occurring ribo- and deoxyribo-nucleosides and nucleotides of all kinds.

The terms “modified nucleoside” and “modified nucleotide”, and “nucleoside analogs” and “nucleotide analogs” are used interchangeably, and refer to chemically modified, non-naturally occurring nucleosides and nucleotides of any kind. The chemical modification may, for example, be at the base, sugar, and/or linkage portions of a nucleotide or nucleoside. Examples of such analogs include without limitation methylphosphonate nucleotide analogs, phosophorothioate nucleotide analogs, peptide nucleic acid (PNA), locked nucleic acid, 2′-halo-modified nucleotides, 2′-alkyl-modified nucleotides, as well as other modified nucleosides and nucleotides

The terms “nucleoside substitute” and “nucleotide substitute” refer to one or more chemical compounds of any kind which are not naturally occurring or modified nucleoside or nucleotide compounds but which are incorporated in the backbone of a oligomer. Examples include sugars, and peptides, amino acids, and lipids, as well as other chemical compounds and combinations of these compounds. One of skill in the art will be aware that a large number of different modified and substitute nucleoside and nucleotide compounds exist which may be suitable for use in the invention.

Unless clearly indicated to the contrary, the terms “nucleotide oligomer”, “oligonucleotide”, and “oligomer” are used interchangeably to refer to covalently linked chain molecules at least 4 chain moieties in length, which principally contain nucleotides and/or nucleotide analogs in the backbone of the chain, but which may also contain one or more nucleotide substitutes (generally a relatively small number) such as sugars and the like in the chain backbone. Such oligomers may be up to 400 chain moieties (e.g., nucleotides or nucleotide analogs) in length, e.g., 6-25, 10-30, 15-50, 10-50, 25-50 50-100, 100-150, 150-200, 200-300, 300-400 chain moieties.

As used herein, the terms “oligomer preparation” and “nucleotide oligomer preparation” are used interchangeably to refer to set or population of chemically synthesized chains principally containing nucleotides and/or nucleotide analogs, but which may also contain one or more nucleotide substitutes. In most cases, the set will include a large number of such chains, e.g., at least 100, 1000, 10,000, 1,000,000, 10⁷, 10⁸, 10¹⁰, or 1 picomole, 1 nanomole, 1 micromole, 1 millimole, 1 mole, or more, even much more.

In the present context, the term “oligomer application” and like terms refer to a process which includes the direct or indirect use of an oligomer(s), information about such oligomer(s), or information or results produced directly or indirectly from a method using such oligomer(s) or information or results to produce particular information and/or results and/or compositions. Thus, an “improved oligomer application” is one which is better than a reference method in at least one characteristic, e.g., produces higher quality information and/or results and/or compositions, and/or is faster and/or easier to perform without sacrificing the quality of the information and/or results and/or compositions.

Throughout this description in connection with populations of oligomers, the terms “preparation” and “prep” are used synonymously to refer to such populations. In many cases, but not all, such a population is the result of a single synthesis process. Alternatively, such a population may be created by combining products from multiple synthesis and/or purifications, and other such sources. The preparation be at various stages, e.g., it may be a crude preparation, a partially purified preparation, a highly purified preparation, and the like.

In the context of oligomer synthesis, the term “protective group” has its convention meaning, referring to groups attached to a moiety during or in preparation for the synthesis process to prevent unwanted reactions from occurring, e.g., during subsequent coupling steps or to prevent addition of additional sub-units. Normally such protective groups are removed or reacted to remove the group or modify it to a desired product (e.g., using particular chemical or light exposure conditions).

The term “purify” in relation to a mixture of different molecules refers to a process of removing at least some of the molecules from the mixture containing a desired molecule or set of molecules being purified. In many cases, solute not considered as part of the mixture being purified. The result is that the desired molecule of molecules will constitute a greater proportion of the molecules in the purified product mixture than in the original mixture. Thus, “purified oligomer preparation” and “purified desired preparation” refer to a mixture or preparation which contains respectively oligomer or desired chain, and which contains a reduced fraction of other molecules which were previously present. For example, such purification may remove some or all of the other synthesis components and/or chains which do not have the intended length or contain other damage.

In the context of an oligomer application, the term “result” refers to the actual data or some aspect of the actual data which is directly generated by the practice of the application. As an example, the direct use of invention improved lower order application data in a higher order application will produce higher order application data which is improved in one or more aspects, such as accuracy or quantitation or reproducibility. In the same oligomer application context, the term “information” refers to some aspect of the conclusions (which may be overall conclusions) reached from or based on the application results or data. As an example, the use of invention improved lower order application results in a higher order application will produce higher order application information which is improved in one or more of interpretability or intercomparability or reliability or utility or predictive power.

In the context of oligomer synthesis and applications, the term “solid phase medium” refers to a material which is solid phase under the relevant conditions and to which oligomers can be directly or indirectly stably attached. Examples are well known, and include, for example, plastics (e.g., plates, slides, and chips), glasses (e.g., plates, slides, and chips), silicon chips, filters of various materials, and beads or other particles of various materials or combinations of materials.

Additional embodiments will be apparent from the Detailed Description and from the claims.

### BRIEF DESCRIPTION OF THE DRAWINGS

FIG. 1: Pseudo-First Order hybridization kinetics of P³² dA₃₅+unlabeled excess 7.8×10⁻¹¹ M dT₃₅ in 0.135 M Na⁺@ 25° C. The half-time of association is 40 minutes and the Kₐ=3.69×10⁶ M⁻¹ seconds.

FIG. 2: Hypothetical Dissociation Kinetic (DK) Profile for a homogeneous oligomer duplex population. Here the t.5d=1 m.

FIG. 3: Hypothetical Dissociation Kinetic (DK) Profiles for two different homogeneous oligomer duplex populations measured under the same dissociation solution and temperature conditions.

FIG. 4: Hypothetical Dissociation Kinetic (DK) Profile for a heterogeneous oligomer molecule population consisting of equal moles of Oligomer Duplex A (t.5d=3 m) and Oligomer Duplex B (t.5d=20 m).

FIG. 5: Dissociation Kinetic (DK) Profile of P³² dA₃₅-dT₃₅ duplexes in HDB at 50° C. The t.5d=14 m. The straight-line portion of the DK profile represents the Slowly Dissociating Duplex Fraction (SF).

FIG. 6: Dissociation Kinetic (DK) Profile of isolated Stringent Bound Fraction (BF) P³² dT₃₅-dA₃₅ molecules in HDB at 50° C. BF was not dissociated and re-hybridized before DK Analysis. The % SF=98% and the SF t.5d=15 m.

FIG. 7: As in FIG. 6, except DK Analysis was performed in HDB at 48.8° C. The % SF=97% and the SF t.5d=62 m.

FIG. 8: Dissociation Kinetic (DK) Profile of P³² dT₃₅-dA₃₅ Duplex Bound Fraction (BF) and Unbound Fraction (UF) after denaturing each fraction, adding an excess of unlabeled dA₃₅ and hybridizing by annealing. DK Analysis was performed in HDB at 48.8° C.

FIG. 9: Dissociation Kinetic (DK) Profile of Stringent P³² dT₃₅-dA₃₅ UF Duplex molecules from FIG. 8. DK Analysis was performed in HDB at 45° C.

FIG. 10: Dissociation Kinetic (DK) Profiles of: [+] SF for P³² dT₃₅-dA₃₄C₁ Duplex with one C/T base pair mismatch at Position #9 from end of duplex; [×]Normalized DK Profile for the FF P³² dT₃₅-dA₃₅ Duplexes from FIG. 9. DK Analysis was performed in HDB at 45° C.

FIG. 11: Dissociation Kinetic (DK) Profiles of: [+] SF of an N=24 Biological Sequence (BS) Duplex with one C/U mismatch at Position #13; [×] SF of an N=24 Biological Sequence (BS) Duplex with two C/U mismatches at Positions #13 and #14. DK Analysis was performed in HDB at 52° C. The SF of a perfectly matched N=24 BS is equal to about 14,800 m.

FIG. 12: Dissociation Kinetic (DK) Analysis of 1× and 2×HPLC-Purified dT₃₅ preps in HDB at 50° C.: [●] 1×Purified; (Δ) 2×Purified

FIG. 13: Dissociation Kinetic (DK) Analysis of Small (SS) and Large (LS) Scale Synthesis Preps of an N=55 Oligomer in 0.15M Na⁺ at 70° C.

### DETAILED DESCRIPTION OF THE PREFERRED EMBODIMENTS

In order to assist the reader, the following Contents section sets out the order and headings of major sections of this description.

Contents
Presently Preferred Prior Art Method for Analysis and Characterization of Oligomer Molecules and Oligomer Duplex Molecules by Nucleic Acid Hybridization Analysis and Nucleic Acid Hybridized Duplex Disassociation Analysis
P³² Labeling of Oligomers
Determination of Optimum Conditions for the HA Separation of Hybridized P³² Oligomer Duplexes from Non-Hybridized P³² Oligomer Strands
Determination of the Oligomer Molecule Population FH Value
Determination of P³² Oligomer Hybridization Kinetics
The Nature of the Hybridized Duplex Dissociation Process and the Kinetics of Duplex Dissociation
Determination of the Dissociation Kinetic Profile t.5d and % SF Values for Hybridized P³² Oligomer Duplex Molecules
Interpretation of Chemically Synthesized P³² Oligomer Duplex Preparation DK Analysis Results
Isolation and Characterization of P³² Oligomer Prep Sub-Fractions Which Are Enriched for SF P³² Oligomer or FF P³² Oligomer
Estimating the Extent of Damage Associated with the N Oligomer FF Molecules
The Determination of Whether for a Synthesized Purified N Oligomer Molecule Population the Measured Physical-Chemical Properties are Equal to the Intended Physical-Chemical Properties
Measurement of the Effect of Mismatched, Unpaired, or Damaged Nucleotides on the Oligomer Functional Characteristics
Consequence of the Invalidity of the Tacit Prior Art Duplex OM Analysis Method Assumption That the Analyzed Duplex Population is Homogeneous
Effect of Oligomer Covalent Association with Chemical and Biological and Other Molecule Groups, Including Ligands and Receptors, On the Oligomer Functional Characteristics
Alternative Methods for Determining Oligomer Functional Characteristics
Producing Improved Oligomer Functional Characteristic Results for a Variety of Different Purposes. Various Practices of the Invention
The Practice of the Invention for the Purpose of Detecting Single Polynucleotide Polymorphisms in DNA or RNA
Application of Basic Invention Rationale for Determining the Functional Homogeneity and Functional Characteristics of Triplex or Quadriplex Nucleic Acid Molecules
Application of Basic Invention Rationale for Determining the Functional Homogeneity and Functional Characteristics of Nucleic Acid Duplex, Triplex, and Quadriplex Molecules which are in the Presence of Other Small or Large Biological and/or Non-Biological Molecules
The Practice of the Invention for the Characterization of Oligomers Which Are Immobilized On a Surface
Application of the Basic Invention Rationale for Determining the Functional Homogeneity and Functional Characteristics of Non-Nucleic Acid Chemically and Biologically Synthesized and Other Molecules
Computer Implementation of Methods for Determining Improved Oligomer Functional Characteristic Values, Thermodynamic Values, Oligomer Selection, and Oligomer Application Optimization

Definitions

This description uses a large number of terms repeatedly. For simplicity, a number of the terms are abbreviated as shown in the table listing below. These abbreviations are used throughout the description.

| | |
|---|---|
| ABS (Abasic Site) | Designates a phosphate site in a polynucleotide molecule which lacks |
| | an attached base |
| BMC | Designates a molecular complex, which consists of two separate |
| (BiMolecular | molecules. A BMC can be comprised of all nucleic acid molecules, all |
| Complex | non-nucleic acid molecules or combinations of nucleic acid and non- |
| | nucleic acid molecules |
| BS | Designates an oligomer or nucleic acid which has a nucleotide |
| (Biological | sequence which is biological in origin, and is not a homopolymer |
| Sequence) | |
| Cerenkov Counting | The detection and quantitation in aqueous solution of Cerenkov |
| | radiation photons from P³² and other isotopes. |
| CPM | Counts per minute, a measure of the amount of radioactivity in a |
| | sample |
| Damaged Oligomer | An oligomer molecule whose nucleotide length and/or nucleotide |
| Molecule | sequence, and/or nucleotide composition, differs from that of the |
| | intended oligomer nucleotide length, nucleotide sequence, and/or |
| | nucleotide composition. |
| Depurinated | An oligomer molecule, which contains an abasic site caused by the loss |
| Oligomer | of a purine base. |
| DK | The kinetics of dissociation of an oligomer or nucleic acid duplex, |
| (Dissociation | triplex, or quadriplex molecule or of some other non-nucleic acid or |
| Kinetics) | partially nucleic acid BMC or TMC molecule. |
| DK Profile | Designates a dissociation kinetic curve, which is obtained for an oligo- |
| | target duplex, or a BMC or TMC of any kind. |
| FD | Designates the quantitative maximum amount of hybridization |
| | associated with an oligomer prep under DK analysis conditions. In |
| | other words, the measured FH value under DK analysis conditions. |
| FF | Designates the fraction of an oligomer duplex preparation, which |
| (Fast Fraction) | dissociates faster than other oligomer duplexes in the same oligomer |
| | duplex prep. A FF has a smaller t.5d value and a larger kd value than |
| | the slower dissociating fractions. |
| FH | Designates the quantitative value for the maximum fraction of an |
| (Fraction | oligomer prep, which can specifically hybridize, with an excess of |
| Hybridized) | complementary target molecules. |
| HA | Hydroxyapatite, a calcium phosphate crystalline compound. |
| HDB | Designates a specific buffer solution, which is used for the DK analysis |
| (Homopolymer | of homopolymers. The buffer consists of 0.09 m phosphate buffer (PB) |
| Dissociation buffer) | pH 6.8, 0.01% lithium lauryl sulfate, and a total sodium concentration |
| | of 0.135M. |
| HS | Designates a polynucleotide molecule, which is composed of only one |
| (Homopolymer | particular nucleotide. |
| Sequence) | |
| ka or Ka | Designates the association constant for a hybridization reaction or a |
| | BMC formation reaction. Measure in units of M⁻¹s⁻¹. |
| kd or Kd | Designates the dissociation constant for a nucleic acid dissociation |
| | reaction or a BMC or TMC dissociation reaction. Measured in units of |
| | sec⁻¹. |
| MM (MisMatched) | Designates a base pair site in the double strand portion of a nucleic acid |
| | duplex molecule where the base pair is not a complementary base pair. |
| | Such a mm site lowers the stability of the overall duplex. Also |
| | designates a nucleic acid molecule, which does not form perfect base |
| | pair matches with another complementary nucleic acid molecule in the |
| | double strand region of the hybridized duplex. |
| N | Designates the intended nucleotide length of a chemically, |
| | enzymatically or biologically synthesized oligomer molecule or other |
| | nucleic acid molecules. |
| N Oligomers | Designates an oligomer molecule, which has the intended nucleotide |
| | length. A synthesized oligomer prep is often comprised on N oligomer |
| | molecules, N + X oligomers, and N − X oligomers. |
| N − X or N + X | Designates an oligomer molecule, which is shorter or longer in |
| Oligomers | nucleotide length respectively than the N oligomer molecules. X |
| | designates the number of nucleotides longer or shorter than the N |
| | oligomer molecules. |
| OM | Designates the standard optical Tm method, which is commonly used |
| (Optical Tm Method) | to determine oligomer-target duplex equilibrium constants and |
| | associated thermodynamic property values. |
| PM | Designates a base pair site in the double strand portion of a nucleic acid |
| (Perfect Match) | duplex molecule where the base pair consists of perfectly |
| | complementary bases. Also designates a nucleic acid molecule, which |
| | forms perfect base pair matches in the double strand region when |
| | hybridized with a complementary nucleic acid molecule. |
| SF | Designates the fraction of an oligo duplex preparation, which |
| (Slow Fraction) | dissociates slower than any other duplex fraction. A SF has a larger |
| | t.5d and a smaller kd value than the FF or FFs. |
| t.5d | Designates the time required for one half of a homogeneous population |
| | of oligomer duplex molecules to dissociate. t.5d is measured here in |
| | minutes. The t.5d = (0.69 ÷ kd). |
| TMC | Designates a molecular complex, which consists of two separate |
| | molecules. A TMC can be comprised of only nucleic acid molecules, |
| | only non-nucleic acid molecules, or a combination of nucleic acid and |
| | non-nucleic acid molecules. |

The invention relates to essentially all prior art natural and modified RNA and DNA and modified RNA or DNA and other oligonucleotide nucleic acid preps produced by biological means or chemical synthesis means and their use in an intended oligomer application.

The field of application of the present invention is very broad and includes any prior art application of any kind, which utilizes biologically, or enzymatically or chemically synthesized nucleic acid oligomers. Such applications include, but are not limited to any prior art and future oligomer applications in the following areas. Basic and applied and service and commercial and industrial and academic, research and development, product research and development, manufacturing research and development, process research and development, information generation research and development, marketing research and development, sales research and development. Examples of such areas follow. (i) Human, veterinary, botanical, agricultural including all aspects of aqua and marine agriculture, areas relating to pharmaceutical, anti-biotic, pesticide and plant control agents, basic research, discovery, validation, characterization, toxicity, ecological impact, product scale up, manufacturing, prescription and use, disease prognosis, quality control and assurance, marketing and sales. (ii) Gene expression measurement and comparison assays including microarrays and PCR assays of all kinds. (iii) Commercial and non-commercial nucleic acid hybridization or nucleic acid primed diagnostic tests of all kinds. (iv) Mutation detection assays of many kinds. (v) Nucleic acid amplification methods of all kinds. (vi) cDNA synthesis methods of all kinds. (vii) cRNA synthesis methods of all kinds. (viii) Synthetic gene production methods of all kinds. (ix) Synthetic RNA production methods of all kinds. (x) Nucleic acid aptamer synthesis and characterization and use. (xi) Ribozyme design, synthesis, characterization and use. (xii) Sense and anti-sense RNA and DNA design, synthesis, characterization and use, including all forms of small regulatory RNA molecules. (xiii) Gene and cDNA cloning methods. (xiv) Basic and applied research on the physical chemical characterization and properties of oligomers and other nucleic acids which are natural or modified. (xv) Nucleic acid probe, nucleic acid primer, nucleic acid target, and other nucleic acid application design and characterization methods which are used for the rational design of oligomers and nucleic acids for various applications. (xvi) Software applications for the design of an oligomer for use in any oligomer application. (xvii) Any application, which utilizes invention, produced improved oligomer information or results or oligomer application results as part of a further application where the further application results will be improved because of the use of the improved oligomer information and/or results and/or the improved oligomer application information and/or results.

An example is the use of improved oligomer information or results to improve oligomer application gene expression analysis results, which are then used to improve the results of a data mining or systems biology analysis. Another example is the use of improved oligomer information or results to improve the oligomer application optical melt Tm analysis results so that they are more correct and accurate and interpretable, and then using these improved oligomer application optical melt results to produce correct or more correct thermodynamic parameter results, and then said improved thermodynamic results are utilized for the application concerning the understanding of a drug-nucleic acid interaction to produce improved results for this application, and then these improved drug-nucleic acid interaction results are used to develop an improved drug which gives improved performance results.

Specific application areas, which are commonly associated with oligomer preps of all kinds, include, but are not limited to, the following. (i) Genetics, gene cloning and genetic modifications and genetic analysis and gene expression and detection of gene mutations. (ii) Amplified and non-amplified nucleic acid analysis and detection and quantification. (iii) Diagnosis for infectious organisms of all kinds. (iv) Nanostructure assembly and analysis.

The very broad field of application of the present invention also includes any chemically synthesized or biologically synthesized or other molecule, which interacts with another molecule to form a dissociable bi-molecular complex. This includes protein·protein and protein·hapten, antibody·antigen and antibody·hapten bi-molecular complexes of all kinds.

The invention will be described in terms of an exemplary method (which in some cases may be the presently preferred method) for practicing the invention. This method, the HA method using P³² labeled oligomers, is discussed below. Unless otherwise noted, each oligomer molecule preparation used in the description consists of a gel purified or HPLC purified N oligomer preparation in which all or essentially all of the oligomer molecules have the intended nucleotide length N. Individual oligomer preps were produced by commercial and research institutions. Note that this presently preferred method is only one of many different prior art methods which can effectively be used to detect and quantitate the amount of single strand and duplex in a sample. Prior to describing the present invention, it will be useful to discuss the prior art knowledge of the actual and intended functional homogeneity and functional characteristics of prior art produced and purified N oligomer preps.

The vast majority of prior art chemically synthesized oligomers are designed for an intended use which requires the specific interaction or hybridization of the oligomer with an intended complementary nucleic acid target molecule. The intended function of the synthesized oligomer is to specifically recognize and hybridize with an intended target complementary nucleic acid strand to form an oligomer·target duplex molecule which has the required and intended functional characteristics. A synthetic oligomer preparation which is functionally optimal has the following characteristics. (a) All of the oligomer molecules in the prep can specifically recognize an intended complementary target molecule and hybridize with it to form an oligomer·target duplex. (b) All of the oligomer·target duplexes formed have the same duplex stability. (c) The base paired regions of the oligomer·target duplex molecules have the intended perfection of base pair matching. A functional characterization of an oligomer preparation which has optimal functionality should produce the following results. (i) Essentially all of the oligomer molecules can be shown to be able to form specific oligomer·target duplexes under the intended use conditions. (ii) Essentially all of the oligomer·target duplexes have the same stability under the intended use conditions. Generally, but not always, it is intended that the oligomer·target duplexes be stable for hours to days under the intended use conditions. (iii) Essentially all of the oligomer·target duplexes have the intended degree of base pair matching perfection in the double stranded or duplex region of the oligomer·target duplex molecule under the intended use conditions. Generally, it is intended that all of the oligomer·target duplex molecules have the same degree of base pair matching perfection in the duplex region. Prior art oligomer synthesis, purification, and use practice does not determine or know whether particular prior art produced and purified N oligomer preps have optimal functional characteristics or not. Further, said prior art practice does not determine or know whether for the same oligomer, different preps of the purified N oligomer are identical with regard to the above described functional characteristics (i)-(iii).

The just described key characteristics of an optimally functional oligomer prep indicate that such an optimal oligomer prep must consist of a homogeneous population of N oligomer molecules which have the same intended physical chemical properties. A basic requirement for an optimal oligomer prep then, is that it must be a homogenous oligomer prep and be composed of a population of oligomer molecules which all have the same physical chemical properties. At a minimum then, a functionally optimal oligomer prep is homogeneous. Prior art produced and purified N oligomer preps which are not homogeneous, therefore, cannot be functionally optimal oligomer preps, unless they are specifically designed to be heterogeneous. The greater the degree of the N oligomer prep non-homogeneity, the greater the deviation of the N oligomer prep from optimal functionality.

Practically, in order to evaluate whether a prior art synthesized and purified N oligomer prep has optimal functionality, the following must be determined. (i) Whether the N oligomer prep is a homogenous prep consisting of N oligomer molecules which all have the same physical chemical properties. (ii) Whether the N oligomer physical chemical properties are functionally optimal for the intended use. (iii) Whether the intended N oligomer physical-chemical properties are the same as the N-oligomer physical-chemical properties associated with optimal functionality for the intended use. Prior art oligomer synthesis, oligomer purification, and oligomer characterization practice, does not determine or know any of the items (i)-(iii), even for populations of oligomer molecules which are known to have the intended N value. Item (i) will be discussed below. Items (ii) and (iii) will be discussed later.

Practically, in order to evaluate whether a prior art synthesized and purified N oligomer prep has optimal functionality it is necessary to determine the functional characteristics of the N oligomer prep. This is done by determining for the prep the FH, FD, kₐ, and t.5d functional characteristic values for the N oligomer of interest. The determination of the t.5d values requires producing a DK profile for the oligomer. Determination of these values is discussed below.

The description of the invention which follows is presented almost exclusively in terms of the analysis of chemically synthesized oligomers. One of skill in the art will recognize that the basic analysis principles and methods can be readily modified and used for biological oligomers and enzymatically synthesized oligomers and nucleic acids in general, as well as non-nucleic acid molecules.

Presently Preferred Prior Art Method for Analysis and Characterization of Oligomer Molecules and Oligomer Duplex Molecules By Nucleic Acid Hybridization Analysis and Nucleic Acid Hybridized Duplex Dissociation Kinetic Analyses.

As discussed earlier, a variety of prior art methods are suitable for determining for a synthesized oligomer preparation the FH and FD values, and the dissociation kinetic profile for the hybridized oligomer duplexes produced from the synthesized oligomer prep. The presently preferred method for doing these analyses is the well-known hydroxyapatite method, herein termed the HA method (30,33). The HA method can be readily modified for determining the hybridization kinetics, the FH value, the dissociation kinetics, and the FD value, for a synthetic oligomer prep and the oligomer duplexes produced from the oligomer prep. In addition, the HA method can be used to quantitatively separate oligomer single strands from oligomer duplexes or triplexes and quantitatively recover them. This ability greatly enhances the analysis and characterization of the oligomer prep.

For technical reasons it is desirable to analyze oligomer molecules which are labeled in such a way that very small and very large amounts of the analyzed oligomer can be readily detected. It is also desirable to label the oligomer in such a way so that the presence of the label itself has as little effect on the intrinsic physical-chemical properties of the labeled oligomer molecules as possible. Further, it is desirable to use a label which can be readily detected without noticeably affecting the integrity of the labeled oligomer molecule population being analyzed. The radioactive isotope phosphorous −32 (P³²) or P32, fulfills these criteria almost perfectly. Standard prior art methods exist which allow the easy attachment of one P³² molecule to the 5′ end of a large fraction of the oligomer molecules being labeled. After labeling, essentially all of the oligomers in the labeled oligomer prep possess a 5′ P³² or 5′ P31¹ molecule. Thus, each oligomer in the P³² labeled oligomer has the same 5′ charge density, while some oligomer molecules possess a P³² molecule and others a P³¹ molecule. Such an isotopic difference is not believed to affect the physical-chemical properties of the oligomer molecules. Other attributes of analyzing P³² labeled oligomers follow. (a) P³² can be readily and accurately detected and quantitated in aqueous solutions, including all buffer solutions used for hybridization and dissociation HA analysis, by Cerenkov counting (35). No additives are required for such detection. (b) Because of a), P³² labeled oligomer analyzed fractions can be readily analyzed and quantitated for P³² under conditions where the P³² oligomer retains its physical-chemical integrity and functional integrity. (c) Because of b), the analyzed P³² labeled fractions can be re-analyzed and recovered multiple times. (d) Because of c), the quality and scope of the hybridization and dissociation kinetic analyses is greatly improved and expanded.

The HA method is effective for long and short natural and modified oligomers. The basis for the HA methods ability to distinguish between single strand and duplex oligomers and other nucleic acids is extensively discussed in the literature. The conditions for the double vs single strand separation vary for oligomers of different nucleotide length, nucleotide sequence and nucleotide composition, and the appropriate conditions often must be determined empirically by adjusting the composition and temperature of the analysis solution. An HA fractionation solution is usually composed of a phosphate buffer and a detergent, and at times other additives. HA analysis of longer oligomer duplexes generally requires higher phosphate and detergent concentrations and higher temperatures. One of skill in the art will readily recognize the need for such adjustments and the methods for making them.

Note that as discussed earlier, other prior art methods and labels can be used effectively to determine the hybridization and dissociation kinetic characteristics of oligomers and oligomer duplexes. In addition, internal or external labeling sites other than the oligo 5′ end can be used for the analysis of oligo preps.

P³² Labeling of Oligomers.

Oligomers were 5′ end labeled using high specific activity P³² dATP and a standard polynucleotide kinase labeling procedure (34). Immediately after the label step, care was taken to inactivate the polynucleotide kinase before purifying the labeled oligomer. Non-reacted P³² ATP was removed by a combination of sephadex chromatography and ethanol precipitation in the presence of highly purified glycogen. The P³² specific radioactivity (SRA) value was measured in terms of Cerenkov counts per minute (CPM) per picomole (Pm) of input oligomer. The counting efficiency of P³² in the scintillation counter used was about 40 percent. The P³² oligomer SRA was generally near the maximum possible of around 4-5×10⁶ CPM/Pm. Generally, the SRAs ranged from 10⁶ to about 5×10⁶ CPM/Pm. Each P³² oligomer prep was characterized for the fraction of P³² not associated with an oligomer molecule by DE-81 binding, HA binding, or gel analysis. Typically, such a fraction constituted 1% or less of the total P³² oligomer prep. This was also checked each time the P³² oligomer prep was used for analysis. The P³² oligomer preps were stored at 4° C.

Note that only one P³² molecule is attached to an oligomer molecule, and that P³² molecule is attached to the 5′ end.

Determination of Optimum Conditions for the HA Separation of Hybridized P³² Oligomer Duplexes from Non-Hybridized P³² Oligomer Single Strands.

The basis for the ability of the HA to separate double and single strand nucleic acids is believed to be the difference in charge density of the double strand hybridized oligomer duplex molecules and the non-hybridized single strand oligomer molecules. Because of the higher duplex negative charge density, the duplex oligomer has a much higher binding affinity for an HA crystal than does the non-hybridized oligomers. Inorganic phosphate will compete with the oligomer duplex and the non-hybridized oligomer for HA binding sites. At a particular inorganic phosphate concentration in the separation buffer, the oligomer duplex molecules adsorb quantitatively to the HA, while all or most of the non-hybridized single strand oligomer does not bind to the HA and passes thru the HA column, where it can be collected and measured. The binding of the single strand oligomer to the column is temperature dependent, and at lower temperatures, a higher inorganic phosphate concentration is required to prevent the single strand oligomer binding. The single strand oligomer binding to HA is also dependent on the oligomer secondary structure. Strong oligomer molecule secondary structure is often associated with oligomers with a high G+C composition of 60-70%. Such high G+C oligomer single strands often require a higher inorganic phosphate concentration to prevent HA adsorption, than do lower G+C oligomers. The single strand binding to HA is also nucleotide length dependent. A higher inorganic phosphate concentration is required for longer oligomers.

For the quantitative measurement of the hybridization of P³² oligomer with a complementary nucleic acid, a buffer solution composition and temperature of fractionation combination which promotes the essentially complete binding of P³² duplex to the HA and which prevents essentially all P³² non-hybridized oligomer from binding to the HA, can almost always be readily and quickly determined by experimentation. The use of Cerenkov counting greatly facilitates this process.

Once bound to HA, the P³² duplex can be recovered quantitatively by washing the HA with a buffer containing 0.24 to 0.3M inorganic phosphate. Once recovered, the P³² duplexes can be diluted to the lower phosphate concentration of the separation buffer, and re-passed over the column. When this is done essentially all of the P³² duplex rebinds to the HA. Similarly, when the non-binding single strand P³² oligomer is re-passed over an identical column, essentially all of it again passes thru the column. Note that at the end of a fractionation after the high phosphate wash, the HA bed can be dissolved in 6N HCL and recovered and assayed for radioactivity. This was done routinely for the work reported herein, and only a small fraction of the total P³² analyzed is present in this fraction. Such a step ensures that the entire amount of input P³² oligomer can be accounted for.

Determination of the Oligomer Molecule Population FH Value.

For an oligomer molecule prep, the FH value is equal to the fraction of the oligomer molecules which can hybridize to complementary nucleic acid molecules. For a P³² label oligomer prep, the FH value is equal to the fraction of the P³² labeled oligomer molecules which can hybridize to an appropriate complementary nucleic acid molecule. The presently preferred HA method for determining the FH value for a 5′ P³² labeled oligomer molecule prep is discussed below. Herein, a P³² labeled oligomer molecule is termed a P³² oligomer molecule, and a P³² labeled oligomer prep is termed a P³² oligomer prep.

The general form of the HA based method for determining the FH value for a P³² oligomer prep follows.

(a) Mix a large molar excess of the unlabeled complementary nucleic acid single strand molecules with a small amount of the P³² oligomer molecules of interest, in a hybridization solution whose ionic strength is sufficient to support the formation of stable P³² oligomer molecule complementary strand duplexes. For such hybridization solutions the ionic strength is primarily determined by the sodium phosphate pH=6.8 buffer present in the solution. Such hybridization solutions generally contain 0.08M to 0.12M phosphate buffer at pH 6.8. This represents a Na⁺ concentration of 0.12 to 0.18M. Such hybridization solutions also generally contain a detergent at low, 0.001% to 0.1%, concentrations. Generally, the detergent sarkosyl or lithium dodecylsulfate is used. Other additives such as NaCl, formamide, DMSO, an alcohol, and others may also be present. Generally, the hybridization solution contains a large molar excess of the unlabeled complementary nucleic acid strand over the P³² oligomer. The molar concentration of the unlabeled complementary strand is generally around 5×10⁻⁸M to 10⁻⁷M. For this pseudo-first order hybridization condition, at the temperature optimum for hybridization the halftime of hybridization will generally be around 1 to 50 seconds. The hybridization volume is usually around 0.1 ml, but could be much smaller. The hybridization solution is placed in a sealed container.

(b) The hybridization mixture is then placed in a closed water bath which has a temperature well above the Tm of the oligomer-complementary strand duplex and incubated for 5 minutes or so. Depending on the oligomer, this elevated temperature can range from around 65° C. for an N=35 dT:dA duplex, to 95° C. or so for an N=89 duplex with a biologically relevant nucleotide sequence, or even higher. The water bath is then turned off. This is a classical hybridization annealing process. The hybridization mix passes slowly thru a large number of hybridization stringency conditions as it slowly cools. As an example, for one annealing process which started at 75° C., after the heater was turned off the water bath temperature dropped at a rate of roughly 0.1 to 0.2° C. per minute. This rate is easily adjusted. Generally, the bath temperature is allowed to drift down to room temperature, and the annealing step is often done overnight.

(c) At the end of the annealing step the hybridization mix is diluted into a large volume of the column buffer used for HA fractionation. This is done at room temperature.

(d) An aliquot of the hybridization mix is analyzed using the earlier discussed and well known HA method. Generally, the P³² oligomer containing aliquot in the HA separation solution has a volume of about 1 to 3 milliliters (ml). However, the aliquot may be larger or smaller. The aliquot is put onto a temperature controlled water jacketed column containing a bed of HA equilibrated to the HA separation solution, and column temperature. The aliquot is allowed to equilibrate to column temperature and then passed through the column directly into a scintillation vial. The HA is then washed with separation solution which is equilibrated to column temperature, and also collected in the same scintillation vial. The P³² aliquot and first wash eluates constitute the first HA fraction or HA-F1. The volume of eluate in the HA-F₁ is 5 to 10 ml. The HA is then washed again with temperature equilibrated HA fractionation solution and the eluate collected in a second scintillation vial. This sample is termed HA-F₂, and contains 5-10 ml. At this point the HA-F₁ and HA-F₂ contain the P³² oligomer single strand molecules, and the P³² oligomer duplexes remain bound to the HA column. The P³² oligomer duplexes can be recovered intact by passing duplex elution solution (0.3M phosphate buffer pH 6.8) over the HA bed and collecting the eluate in a third scintillation vial. This sample is termed HA-F₃ and contains 5-10 ml. At this point, essentially all P³² oligomer should have been eluted from the HA column. This can be confirmed by dissolving the HA bed in 6M HCl and collecting the eluate in a fourth scintillation vial. This sample is termed HA-F₄ and contains 5-10 ml. Generally, there is little P³² in HA-F₄. Any P³² present in HA-F₄ it is considered to represent P³² oligomer duplex. An alternative method of recovering the P³² oligomer duplex fraction from the HA column bed is to dissolve the HA in 6M HCl after collecting the HA-F₂, and collecting the HCl eluant in a third scintillation vial, and then washing the column with water. Here, this sample is termed HA-F₃ and contains 5-10 ml. Note that the low pH of the HCl causes the P³² oligomer duplexes to dissociate and also can cause extensive depurination of the P³² DNA. After collection the scintillation vials are cooled to room temperature and assayed in a scintillation counter along with a background control vial. Each vial is counted multiple times. For each sample, the P³² counts per minute (CPM) value associated with the P³² oligomer in the sample, is equal to (the total P³² CPM for the sample)−(the CPM of the background sample). For each P³² oligomer hybridization extent HA analysis, (the percent of the P³² oligomer in the duplex fraction) is equal to, [(the P³² oligomer CPM in F₃+the P³² oligomer CPM in F₄)÷(the total P³² oligomer CPM in F₁+F₂+F₃+F₄)]×100. Alternatively, (the percent of the P³² oligomer in the duplex fraction, is equal to, [(the P³² oligomer CPM in F₃)÷(the total P³² oligomer CPM in F₁+F₂+F₃)]×100. Note that the efficiency of P³² Cerenkov counting in a scintillation counter is volume dependent. Because of this, all fractions should contain the same volume, or each sample's CPM value should be corrected for differences in efficiency. One of skill in the art will recognize that the above described HA analysis method is but one of many possible practical and effective HA based methods capable of measuring the hybridization extent of P³² oligomers. The P³² fraction in the duplex fraction then, is equal to, (the number of P³² CPM present in the duplex fraction)÷(the total number of P³² CPM recovered in the single strand or unhybridized fraction+the total number of P³² CPM recovered in the duplex or hybridized fraction). Typically, the P³² fraction present in the hybridized fraction is greater than 0.95 and is often equal to 0.98 to 0.99 or more. Herein, this fraction is termed the measured hybridized P³² fraction.

(e) The measured hybridized P³² fraction for P³² oligomer contains the P³² oligomer duplexes and any non-oligomer duplex associated P³² which binds to HA in the analysis. For a P³² oligomer prep, the maximum amount of such a non-oligomer duplex associated P³² HA binding fraction can be measured by passing non-hybridized P³² oligomer over the HA column under the same HA analysis conditions. Here, the P³² fraction which is present in the hybridized fraction, represents the maximum amount of the total oligomer P³² which is not associated with oligomer duplex molecules, which can be present in the quantitative value for the HA measured P³² oligomer hybridized fraction. Herein, the HA binding non-oligomer duplex associated P³² fraction is termed the zero time of hybridization fraction or the ZT fraction. The ZT fraction for a P³² oligomer prep generally represents 0.5 to 3 percent of the total P³² oligomer fraction, and is often one percent or lower. However, ZT values of 10 percent or so are occasionally associated with the P³² oligomer prep and the HA analysis system used. Part of the development of an HA analysis system is to reduce the ZT as far as possible without reducing the ability to completely bind the P³² duplex fraction. If the measured P³² duplex fraction is 0.98, and the P³² oligomer prep ZT value is 0.01, then the corrected value for the P³² oligomer hybridized fraction is equal to (0.98−0.01) or 0.97. For the P³² oligomer prep the FH value is then, equal to 0.97. When the P³² associated with the ZT value is not associated with the oligomer, the described correction process is also accurate.

For certain oligomers the zero time binding value is associated with the secondary structure of the P³² oligomer. P³² oligomers with higher G+C contents tend to have higher ZT values. For such a P³² oligomer the P³² ZT binding fraction cannot be quantitatively removed from the P³² oligomer prep by passing the P³² oligomer molecules through the HA column in the HA fractionation solution. Here, when the recovered first pass non-binding P³² oligomer fraction is re-passed over another HA column under identical conditions, the ZT binding values of the first and second pass P³² oligomers are similar. For such a P³² oligomer if the ZT binding is small enough, roughly 1-2 percent or less, this type of ZT binding value can be corrected for as described above when the % hybridization value being corrected is large enough, say 20% hybridization or so. However, for certain P³² oligomers the ZT binding in the HA analysis system may be 10% or so. For such a high ZT binding a different method is required to correct the % hybridization value. This can be illustrated by considering the following P³² oligomer hybridization, HA analysis system. (a) The P³² oligomer can hybridize to 100%. (b) For the HA analysis system used the P³² oligomer ZT binding value is 10%. (c) All individual P³² oligomer molecules are identical.

For this situation, when the ZT binding value is intrinsic to the 32 molecule, then when the HA measured % hybridization is 100%, no ZT correction is needed since none of the P³² oligomer molecules were in a single strand state when contacted with the HA during the analysis. However, if the HA measured hybridization extent is 55%, this value must be corrected in order to obtain the corrected extent of P³² oligomer hybridization. Here, the measured % hybridization is 55%. This means that for this assay 45% of the P³² oligomer passed through the HA column and is non-hybridized single strand P³² oligomer. If for this assay, 45% of the P³² oligomer passes through the HA column, and the ZT binding value for single strand P³² oligomer is always 0.1, then the amount of unhybridized P³² oligomer single strand present in the hybridized P³² oligomer fraction can be determined using the following relationship. Here, (the actual or correct hybridized P³² oligomer fraction value)=1−[(measured non-hybridized fraction value for the P³² oligomer)÷(1−ZT bind fraction value for the P³² oligomer)]. For this example then, (the corrected P³² oligomer hybridization value)=1−(0.45÷0.9) or 0.5. Table 2, oligomer (4) presents results for a P³² 2 OMe oligomer which has such a high ZT binding value.

For certain combinations of P³² oligomer and HA analysis conditions, the ZT binding value will be composed of significant ZT binding which is intrinsic to the P³² oligomer, and also ZT binding which is not directly associated with the P³² oligomer. This latter contribution can often be removed by pre-passing the P³² oligomer through HA under the desired HA analysis conditions before use for the hybridization extent analysis. For a P³² oligomer prep, the relative amounts of each of these contributions to the ZT binding value can be estimated by re-passing the P³² oligomer over HA 3 to 4 times under the desired HA analysis conditions. The ZT binding value for the last pass should reflect the ZT binding due only to the intrinsic properties of the P³² oligomer. One skilled in the art can then use these results to correct measured hybridization extents for ZT binding due to one or both contributory sources.

For a P³² oligomer prep, the FH values obtained are generally above 0.95 and are often equal to 0.98 to 0.99. FH values of around 0.9 are usually associated with P³² N oligomer molecule populations which are significantly heterogeneous. Table 1 presents for various P³² oligomer preparations the HA measured ZT values and fraction of P³² oligomer bound to HA after hybridization values, and the FH values determined from them.

Note that when the hybridized P³² fraction is recovered and the solution adjusted to the composition of the HA fractionation solution, essentially all of the P³² rebinds to HA when re-passed over an HA column.

TABLE 1
Determination of P³² Oligomer Prep FH Values By HA Analysis

| | | | | Fraction of P³² | | |
|---|---|---|---|---|---|---|
| Table 2 | Oligomer Type and | Hybridization Solution | HA Fractionation | Bound to HA | ZT | HA Measured |
| P³² | Length N of P³² | and Starting | Solution and | After HYB | Fraction | FH Value |
| Oligomer | Oligomer | Temperature | Temperature | (%) | (%) | (%) |
| (1) | N = 35 | 0.09M PB pH6.8 | 0.09 MPB PH6.8 | 99.5 | 0.5 | 99 |
| | dA35 | 0.135M Na⁺ Total | 0.01% LLS (25° C.) | | | |
| | | 0.02% LLS (25° C.) | | | | |
| (12) | N = 35 | As (1) | As (1) | 97.1 | 2.7 | 94.4 |
| | dT₃₅ | | | | | |
| (13) | N = 35 | As (1) | As (1) | 99.4 | 0.6 | 98.8 |
| | dA₃₅ | | | | | |
| (2) | N = 35 | As (1) | As (1) | 90.5 | 1.4 | 89.1 |
| | dT35 | | | | | |
| (3) | N = 35 | 0.1M PB pH6.8 | Same as HYB | 94.3 | 2.6 | 91.7 |
| | dC₃₅ | 0.15M Na⁺ Total | Solution (70° C.) | | | |
| | | 0.01% LLS (70° C.) | | | | |
| (6) | N = 24 | 0.09 M PB pH6.8 | Same as HYB | 97.4 | 0.2 | 97.2 |
| | BS DNA | 0.135M Na⁺ Total | Solution (41° C.) | | | |
| | | 0.01% LLS (37° C.) | | | | |
| | | 0.09M PB pH6.8 | 0.09 MPB PH6.8 | 86.1 | 0.1 | 86 |
| | | 1M Na⁺ Total | 0.01% LLS(41° C.) | | | |
| | | 0.01% LLS (59° C.) | | | | |
| (10) | N = 57 | 0.11M PB pH6.8 | Same as HYB | 95.9 | 0.5 | 95.4 |
| | BS DNA | 0.165M Na⁺ Total | Solution (66° C.) | | | |
| | | 0.01% LLS (73° C.) | | | | |
| (11) | N = 89 | 0.018M PB pH6.8 | Same as 10 HYB | 99.6 | 0.4 | 99.2 |
| | BS DNA | 1.02M Na⁺ Total | (55° C.) | | | |
| | | 6 × 10⁻⁴ M EDTA | | | | |
| | | 0.002% LLS (23° C.) | | | | |

BS = Biological Sequence

In addition, when the non-binding P³² fraction from the hybridization analysis is re-passed over HA, essentially all of it re-passes thru the column and remains unbound. The observations indicate that the P³² duplex fraction is quantitatively bound to the HA during the HA analysis.

Determination of P³² Oligomer Hybridization Kinetics.

As discussed earlier, the HA method is the presently preferred method for determining the hybridization kinetics of a P³² oligomer molecule population with its complementary nucleic acid molecules. Such use of the HA method is well known and the discussion in the preceding section concerning the determination of the P³² oligomer molecule population FH value is directly pertinent to the determination of the P³² oligomer hybridization kinetics. The basic hybridization determination process involves the following.

(a) Add a known molar excess or equimolar amount of unlabeled complementary nucleic acid molecules, and a known lesser molar amount or equimolar amount of P³² oligomer molecules to a known volume of hybridization solution, which has a known ionic strength, pH, and composition. In the final hybridization mixture, the molarity of the unlabeled complementary nucleic acid molecules must be known, and the ionic strength and pH and composition of the hybridization solution must be known. The P³² oligomer molecule molar concentration can be significantly less than that for the unlabeled complementary strands. Preferably, a tenfold or greater molar excess of the unlabeled complementary molecules should be present in the hybridization solution. The composition and pH of the hybridization solution used depends on the intended purpose and use of the hybridization kinetic measurements. Place the hybridization mixture in a sealed container.

(b) Incubate the hybridization mixture at the desired temperature of incubation.

(c) At each desired time after the time zero start of the hybridization, remove an aliquot of the hybridization mixture, and dilute it into a large volume of HA fractionation solution. Such dilution should be sufficiently large to prevent the non-hybridized P³² oligomer from hybridizing significantly during the HA assay procedure. Alternatively, the non-hybridized P³² oligomer molecules which are present can be prevented from hybridization during the HA analysis by including an excess of the same unlabeled analyzed oligomer molecules in the dilution solution so that the complementary strand can, in essence, hybridize with only the unlabeled strand. The ratio in the dilution solution of, (the mole amount of unlabeled analyzed oligomer molecules present in the dilution solution)÷(the mole amount of unlabeled complementary nucleic acid molecules present in the dilution solution), should be high enough so that it is not possible for a significant amount of the unhybridized P³² oligomer molecules which are present in the dilution solution to hybridize with unlabeled complementary molecules.

(d) Analyze the diluted fraction on HA at the desired temperature and quantitatively determine the fraction of hybridized and unhybridized P³² oligomer for each time point. The ZT and maximum hybridization extent for the P³² oligomer molecules are determined. The discussion in the preceding section on the determination of the ZT and FH value for a P³² oligomer are directly applicable here.

(e) When the ZT corrected maximum extent of hybridization is less than 100%, each time point corrected hybridization extent value should be normalized to the corrected maximum extent of hybridization for the P³² oligomer. Thus, (the normalized hybridization extent at time t)=the ZT corrected hybridization extent at t)÷(the ZT corrected maximum hybridization extent for the P³² oligomer).

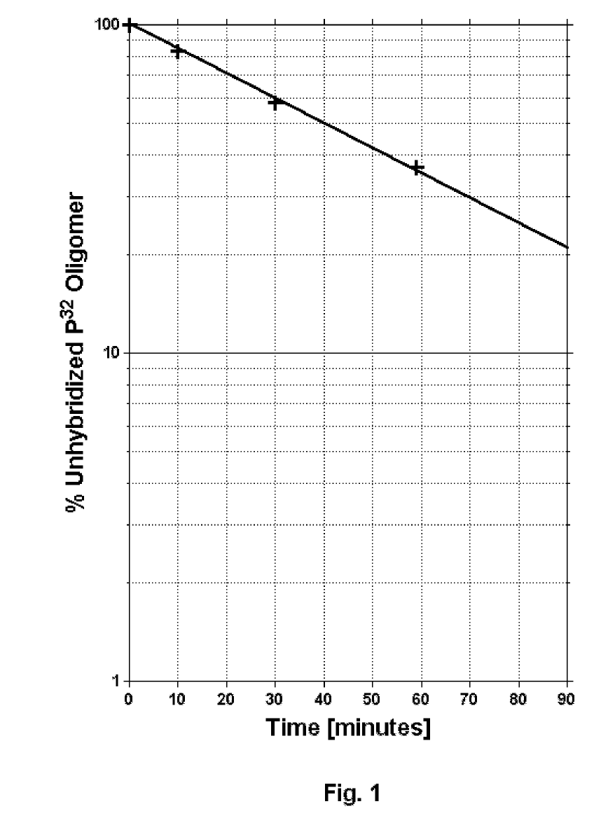

(f) A tenfold or greater molar excess of the unlabeled complementary molecules is present in the hybridization mixture and because of this the kinetics of hybridization of the P³² oligomer with the excess complement should resemble those of a first order kinetic process. While the hybridization kinetics for this reaction have the form of the kinetics of a first order process, the hybridization reaction is concentration dependent, and does not represent a true first order process. Such kinetics are commonly called pseudo-first order hybridization kinetics. Semilog graphing of first order kinetic results is common and will be discussed in more detail later. Here, the log of the fraction of P³² oligomer single strand left versus time, is graphed to display the results. Such a semilog hybridization kinetic graph is presented in FIG. 1.

(g) The time for one-half hybridization of the P³² oligomer under the hybridization condition of excess complement concentration and solution composition and temperature are determined from the graph. The hybridization kinetic pseudo-first order rate constant kₐ, is then determined using the relationship kₐ=(0.693)÷(t.5a) (Co), where t.5a is the measured P³² oligomer molecule hybridization halftime, and Co is the molar concentration of the excess unlabeled complement. For the P³² oligomer hybridization kinetics depicted in FIG. 1, the (Co=7.8×10⁻¹¹M) and the t.5a=(2400 sec), and, therefore, the kₐ=3.7×10⁶M⁻¹ sec⁻¹. These hybridization kinetics are associated with the Table 2 (13) hybridization kinetic analysis of P³²dA₃₅ and unlabeled dT₃₅.

TABLE 2
Measured Hybridization Kinetic kₐ Values for A variety of Different P³² oligomer Molecules

| | | Nature of Complement | | | | | |
|---|---|---|---|---|---|---|---|
| | | Oligomers - Biological | | | | | |
| | | Sequence (BS) | | | | | Corrected |
| | | Perfect Match (PM) | | Hybridization Solution | | Pseudo-First | Maximum |
| P³² Oligomer N | | Mismatch (MM) | | | Hybridization | Order ka | HYB. Extent |
| Value (Nucleotides) | | Nucleotide Length (N) | | Composition | Temperature | (M⁻¹ sec⁻¹) | (%) |
| (1) | N = 35 | N = 35 | dT₃₅ | 0.09M PB | 25° C. | 3.7 × 10⁶ | 99 |
| | dA₃₅ | PM | | 0.135M Na⁺ Total | | | |
| | | | | 0.01% LLS pH 6.8 | | | |
| (2) | N = 35 | N = 35 | 18 | 0.09M PB | 25° C. | 5.5 × 10⁶ | 89 |
| | dT₃₅ | MM | dA34.C1 | 0.135M Na⁺ Total | | | |
| | | | | 0.01% LLS pH6.8 | | | |
| (3) | N = 35 | N = 35 | dG₃₅ | 0.1M PB | 70° C. | 1.3 × 10⁵ | 91 |
| | dC₃₅ | PM | | 0.15M Na⁺ Total | 70° C. | 2.3 × 10⁵ | |
| | | | | 0.01% LLS pH6.8 | | | |
| (4) | N = 22 | N = 22 | BS DNA | 0.05M PB | 40° C. | 8 × 10⁶ | 88 |
| | 2-OMe | PM | | 0.525M Na⁺ Total | 43° C. | 7.6 × 10⁶ | |
| | Oligomer BS | | | 0.002% LLS pH6.8 | | | |
| (5) | N = 24 | N = 24 | BS DNA | 0.08 M PB | 33° C. | 5.6 × 10⁶ | 93 |
| | BS DNA | Perfect | | 1M Na⁺ Total | 42° C. | 6.9 × 10⁶ | |
| | MM (Linker | Biological | | 0.001% LLS pH6.8 | | | |
| | Present in | Sequence | | 0.09 M PB | 33° C. | 8.6 × 10⁵ | 95 |
| | Sequence | | | 0.135M Na⁺ Total | 42° C. | 1.3 × 10⁶ | |
| | Between 15/16) | | | 0.001% LLS pH6.8 | | | |
| (6) | N = 24 | N = 35 | BS DNA | 0.09M PB | 37° C. | 6.4 × 10⁵ | 94.4 |
| | BS DNA | PM | | 0.135M Na⁺ Total | 56° C. | 7 × 10⁵ | 83 |
| | | | | 0.01% LLS pH6.8 | | | |
| | | | | 0.09 M PB | 52° C. | 1.2 × 10⁷ | 91 |
| | | | | 1M Na⁺ Total | 59° C. | 1.5 × 10⁷ | 85 |
| | | | | 0.01% LLS pH6.8 | | | |
| (7) | N = 24 BS | As (6) Complement PM | | 0.09M PB | 29.2° C. | 3.4 × 10⁵ | 92 |
| | DNA As (6) | | | 0.135M Na⁺ Total | 37° C. | 5.5 × 10⁵ | 87 |
| | Except is a C/U | | | 0.01% LLS pH 6.8 | | | |
| | Mismatch at | | | | | | |
| | Positions 13 | | | | | | |
| | and 14 (2 MM) | | | | | | |
| (8) | N = 29 | N = 29 | BS | 0.09M PB pH 6.8 | 25° C. | 5.1 × 10⁵ | 89 |
| | BS DNA | PM | DNA | 0.135M Na⁺ Total | 40° C. | 5.1 × 10⁵ | 92 |
| | | | | 0.01% LLS | 54° C. | ˜5.7 × 10⁵ | 48 |
| | | | | 0.02M Tris pH7.4 | 25° C. | 1.7 × 10⁷ | 87 |
| | | | | 1M Na⁺ Total | 54° C. | 8.5 × 10⁶ | 85 |
| | | | | 6 × 10⁻⁴M EDTA | | | |
| | | | | 0.02 M Tris pH7.4 | 25° C. | 1.8 × 10⁷ | 85 |
| | | | | 7 × 10⁻³M Mg⁺⁺ | 54° C. | 7 × 10⁶ | 87 |
| | | | | 0.02M Tris pH7.4 | 25° C. | 2.4 × 10⁷ | 85 |
| | | | | 1M Na⁺ Total | | | |
| | | | | 7 × 10⁻³ M Mg⁺⁺ | | | |
| (9) | N = 29 | N = 140 Double Strand PM | DNA | 0.1M EPPS pH8.0 | 48° C. | ˜6.2 × 10⁶ | 88 |
| | BS PM | N = 140 Molecule Contains | | 0.5M Na⁺ Total 0.1% | | | |
| | Same Chimeric | the Perfect Complement of | | SDS | | | |
| | Oligomer as (8) | the P³² Oligomer. DS DNA | | 0.1% NP-40 | | | |
| | | Completely Dissociated | | 0.09M PB pH6.8 | 55° C. | ˜3.4 × 10⁶ | 70 |
| | | Before Use. Concentration | | 0.13M Na⁺ Total | | | |
| | | of DS DNA = 9.4 × 10⁻¹¹M | | 10⁻⁴ M EDTA | | | |
| | | in Hybridization Reaction | | 0.01% LLS | | | |
| | | Mixture. The DS DNA to | | | | | |
| | | P³² Oligomer Ratio in | | | | | |
| | | Mixture was ˜3/1. | | | | | |
| (10) | N = 57 | N = 57 | BS | 0.11M PB pH6.8 | 66° C. | 1.5 × 10⁶ | 95 |
| | BS PM | PM | DNA | 0.165M Na⁺ Total | 73° C. | ˜1.5 × 10⁶ | 83 |
| | | | | 0.01% LLS | | | |
| (11) | N = 89 | N = 89 | BS | 0.018M PB pH6.8 | 24° C. | 2.5 × 10⁶ | 98 |
| | BS DNA | PM | DNA | 1.02M Na⁺ Total | 37° C. | 6.6 × 10⁶ | 98 |
| | PM | | | 6 × 10⁻⁴M EDTA | 56° C. | 1.6 × 10⁷ | 98 |
| | | | | 0.002% LLS | | | |
| | | | | 1M Na⁺ Total | 24° C. | ˜2.3 × 10⁴ | 97 |
| | | | | 3 × 10⁻⁴M EDTA | 56° C. | ˜1.3 × 10⁷ | 97 |
| | | | | Plus Single Strand | | | |
| | | | | Salmon Sperm DNA | | | |
| | | | | at 100 | | | |
| | | | | Micrograms/ml | | | |
| (12) | N = 35 | N = 35 | dA₃₅ | 0.09M PB pH6.8 | 25° C. | 3.7 × 10⁶ | 94 |
| | dT₃₅ | PM | | 0.135M Na⁺ Total | | | |
| | | | | 0.01% LLS | | | |
| (13) | N = 35 | N = 35 | dT₃₅ | As (12) | 25° C. | 3.5 × 10⁶ | 98 |
| | dA₃₅ | PM | | | | | |

BS = Biological Sequence

Table 2 presents the HA method measured hybridization rate constant ka values for a wide variety of different P³² oligomer nucleotide lengths and nucleotide sequences. In addition, the ka values for the same P³² oligomers at different ionic strengths and/or temperatures are presented. As is indicated in Table 2, the dependence of the P³² oligomer kₐ values on the ionic strength and/or temperature of the hybridization solutions are similar to those reported in the prior art for nucleic acid hybridization in general. While ionic strength affects the kₐ values (the effect is large, as is indicated in the prior art), the temperature effect on the kₐ is considerably less than the ionic strength effect over the temperature ranges measured.

Note that the above-described methods and rationales for the HA method determination of P³² oligomer hybridization kinetics, are directly applicable to the determination of hybridized P³² oligomer duplex dissociation kinetics and will be used for that purpose below.

The above described presently preferred method for determining oligomer ka values is but one of many possible HA and non-HA related prior art methods which can be used to determine an oligomer ka value.

The Nature of the Hybridized Oligomer Duplex Dissociation Process and the Kinetics of Duplex Dissociation.

As discussed above, a homogeneous population of hybridized oligomer duplex molecules consists of oligomer duplex molecules in which all of the nucleotides of one oligomer strand are perfectly base paired with a complementary nucleotide in the other oligomer strand. Alternatively, a homogeneous population of hybridized oligomer duplex molecules is one where each nucleotide in one oligomer strand is paired with the intended nucleotide in the other oligomer strand. Alternatively, a homogeneous population of hybridized oligomer duplex molecules is one where each hybridized oligomer duplex molecule has one or more discrete single strand region and one or more discrete double strand region and the nucleotide sequence and position of each single strand and double strand region is the same in each oligomer duplex molecule present.

For a homogeneous population of hybridized oligomer duplex molecules, each oligomer duplex molecule in the prep has identical physical-chemical properties. In the oligomer duplex dissociation process, a single oligomer duplex molecule composed of two single oligomer strands, undergoes strand separation and is converted to two single stranded oligomer molecules. The rate of oligomer duplex dissociation or strand separation is highly temperature dependent and the rate of oligomer duplex dissociation increases greatly as the temperature increases. The rate of oligomer duplex dissociation is also ionic strength dependent, and the dissociation rate decreases with increasing ionic strength. Hybridized oligomer duplex dissociation will occur at any temperature and at any ionic strength. With enough time, all of the duplexes in a hybridized oligomer prep will dissociate.

The oligomer duplex dissociation process is a probabilistic event and the rate of duplex dissociation is temperature dependent. For a homogeneous population of oligomer duplexes the physical-chemical properties of each oligomer duplex molecule present in the population are the same. Thus, in a homogeneous population of oligomer duplex molecules, each individual oligomer duplex molecule in the homogeneous population has the same probability of dissociating during any particular time period. Thus, during a particular short time period, each individual oligomer duplex molecule in the population has the same probability of dissociating. As a result, during this short time period some of the hybridized oligomer duplexes will undergo dissociation while others remain in the duplex form. The probability of dissociation for an oligomer duplex molecule is an intrinsic property of each individual duplex molecule type and is dependent on the specific physical-chemical characteristics of the oligomer duplex molecule and the solution and temperature environment in which the duplex molecule resides. For any particular ionic strength and temperature condition, the probability of dissociation of any individual oligomer duplex molecule is independent of the overall concentration of oligomer duplex molecules present, or the overall concentration of single strand complementary oligomer molecules which comprise the oligomer duplex. Consequently, the fraction of the oligomer duplex molecule population which dissociates per specific time period is the same over time for a particular condition, and is independent of the hybridized oligomer duplex concentration or the concentration of single strand complementary oligomer molecules.

Under fixed conditions, the fraction of existing hybridized oligomer duplex molecules which dissociate per second is the same over time. Such a situation describes a true first order process. Therefore, the kinetics of the dissociation of each particular type of oligomer duplex molecule, and of a homogeneous population of oligomer duplex molecules will have the characteristics of a true first order reaction process. For such a first order process, the logarithm of the concentration of the duplex will decrease linearly with time. Therefore, when the log of the duplex concentration versus linear time is plotted for a homogeneous oligomer duplex dissociation reaction, the measured dissociation kinetic data points fall on a straight line in the graph. This is illustrated below. For this illustration and, within this document, the total concentration of homogeneous oligomer duplexes or the total concentration of heterogeneous oligomer duplexes at time zero, is expressed in terms of 100% of the duplexes present at time zero. For the dissociation kinetic analysis of a homogeneous oligomer duplex molecule population then, when the log of the percent duplexes remaining versus linear time is plotted, the measured dissociation kinetic data falls on a straight line in the graph.

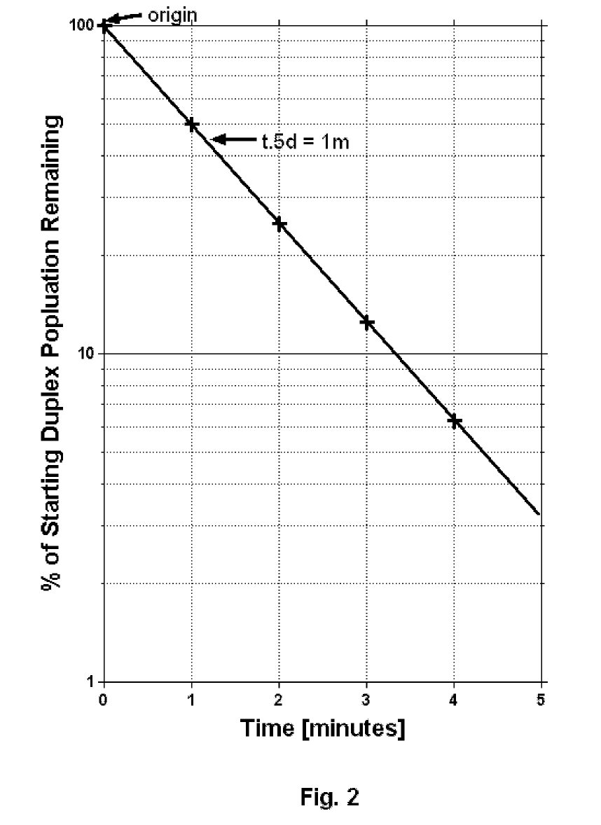

When the measured dissociation kinetics for a homogeneous population of oligomer duplex molecules is plotted in this way, the resulting semilog plot must have the following properties. (a) The data points must fall on a straight line. (b) The straight line must pass through the origin of the graph, which is the oligomer duplex concentration at zero time of the analysis. Herein, the origin for a homogeneous duplex dissociation kinetic analysis is 100% of the duplexes. An example of such a homogeneous oligomer duplex dissociation kinetic plot is presented in FIG. 2. Herein, the dissociation kinetics for a homogeneous oligomer duplex molecule population will be referred to as homogeneous dissociation kinetics and the kinetic plot will be termed a homogeneous Dissociation Kinetic (DK) profile. The only unit associated with dissociation kinetics is time. Commonly, the dissociation kinetics for a particular oligomer duplex are described in terms of the time required for half of the analyzed duplexes to dissociate. Herein, this halftime of dissociation is termed a t.5 value or t.5d value, and the time is measured in minutes or m. For the FIG. 2 illustration the t.5d=1 m.

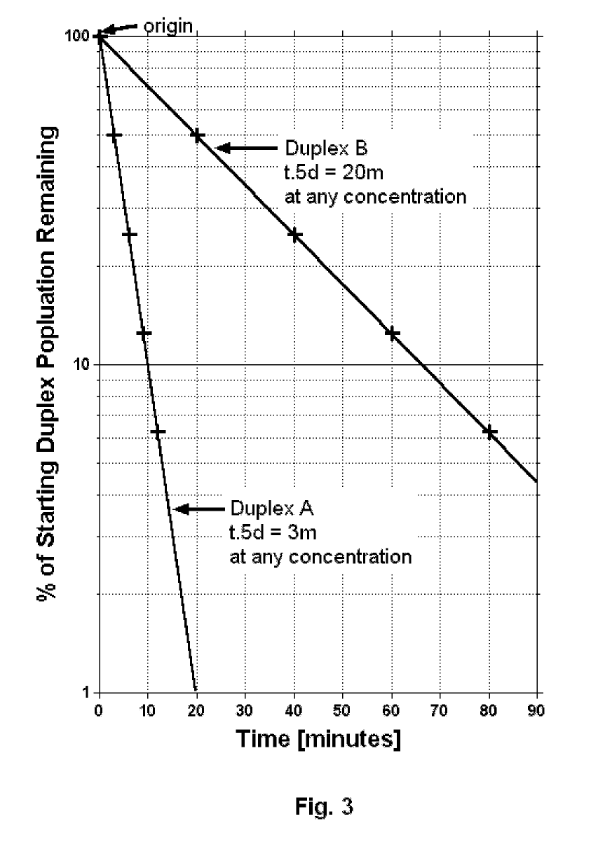

When measured under the same dissociation measurement conditions, homogeneous populations of different oligomer duplex molecules which have different physical-chemical properties will be associated with different t.5d values. This occurs because oligomer duplex molecules which have different physical-chemical properties have different probabilities of dissociating during a fixed time period. Even small differences between otherwise identical oligomer duplexes can result in significant t.5d value differences for the compared duplexes. A variety of factors can cause such dissociation kinetic differences to occur between different homogeneous oligomer duplex molecule populations. These include but are not limited to the following. (a) Different duplex nucleotide lengths. (b) Different nucleotide sequences. (iii) Different natural nucleotide compositions. (iv) Different non-natural nucleotide compositions. (v) Different mismatched base pairs for otherwise identical oligomer duplex populations. (vi) Different unmatched base pairs for otherwise identical oligomer duplex populations. (vii) Different dangling ends for otherwise identical oligomer duplex populations. The difference in the dissociation kinetics for two different but homogeneous oligomer duplex molecule populations is illustrated in FIG. 3. Here, the measured t.5d values for the different homogeneous oligomer duplex molecule populations are 3 m for Duplex A, and 20 m for Duplex B. Here, in FIG. 3 the A and B Duplexes are measured separately. When the A and B Duplexes are mixed together in the same solution and measured under the same dissociation kinetic conditions, the A Duplex t.5d is again 3 m and the B Duplex t.5d is again 20 m.

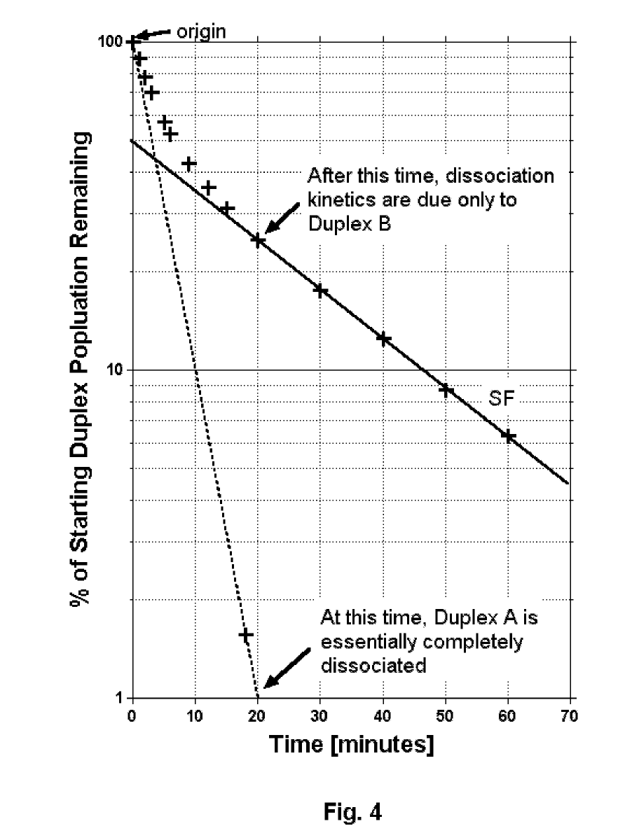

Such a solution represents a heterogeneous oligomer duplex molecule population. The dissociation kinetics of an equimolar mixture of homogeneous A Duplex molecules and homogeneous B Duplex molecules are illustrated in FIG. 4. This mixture represents a heterogeneous oligomer duplex molecule population. For this, and essentially all other heterogeneous oligomer duplex molecule populations, all of the dissociation kinetic data points cannot be connected by one straight line which passes through the origin of the graph. The curved dissociation kinetic profile illustrated in FIG. 4 occurs because the A and B Duplexes dissociate at different rates.

For any oligomer duplex molecule population which is significantly heterogeneous in oligomer duplex molecules with different physical-chemical properties, the semilog graph plotted dissociation kinetics will have the following characteristics. (a) The dissociation kinetic data points do not all fall on one straight line which passes through the origin of the graph, i.e., the 100% duplex present point. (b) The early dissociation kinetic data points are influenced more by the oligomer duplexes which dissociate rapidly relative to the other oligomer duplexes. (c) The late dissociation kinetic points are influenced more by the oligomer duplexes which dissociate slowly relative the other oligomer duplexes. (d) When the slow dissociation data points on the semilog graph can be connected by a straight line, the ordinate intercept percent duplex remaining value for the extrapolated straight line, represents the fraction of the starting heterogeneous oligomer duplex molecule population which is present at zero time of dissociation for the duplexes represented by the straight line. This fraction is equal to (the intercept percent value÷100). This is illustrated in FIG. 4 where the slow dissociation kinetic data points fall on a straight line which extrapolates to the % duplex left value of 50%, and a fraction of the total starting duplex molecules of ( 50/100)=0.5, the actual fraction in the equimolar mixture. Note that in this illustration, at t=20 m about one-half of the slow dissociation oligomer duplex remains, while less than 0.01 of the rapidly dissociating oligomer duplexes remain. After about 20 m then, observed changes in the fraction of oligomer duplexes remaining are, in effect, due only to the dissociation of the slowly dissociating oligomer duplexes, and therefore these later data points then must fall on one straight line. (e) When the slow re-association kinetic data points fall on a straight line which intercepts the ordinate at the percent duplex remaining value of Z, then the t.5d of the slow oligomer duplex fraction is equal to the time value associated with the straight line where the percent duplex remaining is equal to (Z/2). For the Table 4 illustration Z=50%, and for the straight line representing the slow dissociating oligomer duplexes, Z/2 or 25% represents a dissociation t.5d=20 m for the slow oligomer duplex fraction (SF), which is equal to the actual t.5d value of 20 m for the slow dissociating oligomer duplex.

Determination of the Dissociation Kinetic Profile t.5d and % SF Values for Hybridized P³² Oligomer Duplex Molecules.

The determination of the dissociation kinetic profile of hybridized P³² oligomer duplex molecules will be described in terms of the presently preferred method for practicing the invention. This method, the HA method for determining the corrected extent of P³² oligomer hybridization, was described earlier in the context of determining the kinetics of hybridization of P³² oligomer molecules with complementary nucleic acid molecules. This same HA method and rationale for determining the corrected extent of P³² oligomer hybridization, is used to determine the dissociation kinetics of hybridized P³² oligomer duplex molecules, and to fractionate the P³² oligomer duplexes for further analysis. The basic hybridized P³² oligomer duplex dissociation kinetics determination process involves the following. Herein, the dissociation kinetic analysis or determination will be termed DK analysis or DK determination.

(a) Produce hybridized P³² oligomer duplex molecules by mixing a greater or lesser amount of complementary nucleic acid molecules, and a greater or lesser molar amount of single strand P³² oligomer molecules into a volume of hybridization solution which has the desired ionic strength, pH, and composition. In the final hybridization mixture, it is preferred that the molarity of the unlabeled complementary nucleic acids and the P³² oligomer molecules be known. The relative molar amounts present will depend upon the purpose of the DK analysis. The ionic strength, pH, and composition of the hybridization solution also depend on the intended purpose and use of the DK analysis results. Place the hybridization mixture in a sealed container.

(b) Ensure the single strandedness of the P³² oligomer and complementary molecules by heating the solution to a temperature well above the dissociation temperature of the intended duplexes. Incubate at the high temperature long enough to ensure the complete dissociation of any duplexes present.

(c) At this point the hybridization incubation can be done by the earlier described annealing process where the temperature of the hybridization solution is allowed to decrease slowly from the high denaturation temperature, or the hybridization incubation can be done at one temperature for a time long enough to ensure the desired completeness of hybridization. Each method can result in a different quality of P³² oligomer·complement duplex, and the method used will depend on the purpose and use of the DK analysis results. The annealing process is a simple preferred method for producing high quality P³² oligomer complement duplexes which have optimal complementary perfection. This can also be done by the constant incubation temperature method, after determining certain duplex stability information. The time duration of the hybridization incubation may be long or short depending on the purpose and use of the DK analysis results. For determining the DK profile for all of the hybridizable P³² oligomer molecules, the hybridization incubation time must ensure the maximum hybridization of the P³² oligomer molecules with the complement, so that the P³² oligomer FH and/or FD value can be determined. Note that for some purposes the DK FD value will not equal the FH value for the P³² oligomer molecules. At the end of the hybridization period, it is generally preferred to allow the hybridization mixture to equilibrate to room temperature, i.e., about 20 to 25° C.

(d) Dilute an aliquot of the hybridized P³² oligomer duplex molecules into the desired HA fractionation solution in order to determine either or both of the P³² oligomer FH and/or DK FD value. Depending on the purpose and use of the DK analysis results, a different HA fractionation solution may be used for the FH determination than for the FD determination. Often the FH and DK FD values can be determined in the same HA fractionation solution.

(e) Determine the P³² oligomer FH and/or FD values by the earlier described HA method for determining the corrected hybridization extent for a P³² oligomer. As discussed above, this may require the use of two different HA fractionation solutions, one for the FH determination and one for the FD determination, and may also require the use of two different temperatures for the HA fractionation, one for the FH, and one for the DK FD determination.

(f) To conduct the DK analysis, the HA fractionation solution and temperature conditions used for the determination of the DK FD value is used. For this HA fractionation condition the FD value may or may not equal the FH value for the P³² oligomer. This will depend on the purpose and use of the DK analysis results.

(g) Dilute an aliquot of the hybridization mixture into the desired DK analysis solution, which is also at room temperature. The dilution should be large enough to ensure that the ionic strength, pH, or composition, of the dilution solution does not differ significantly from the ionic strength, pH, or composition of the pure HA fractionation solution. This is particularly important for the ionic strength, as it was discovered during the course of this work that the dissociation kinetics of the P³² oligomer duplexes are very sensitive to ionic strength. This is contrary to prior art teaching, as was discussed earlier. Here, the P³² oligomer duplex DK t.5d value is very sensitive to small ionic strength differences in the compared DK solutions. Because of this, care should be taken to ensure that the ionic strengths of compared DK solutions are as similar as possible. This also means that when a new lot of a particular DK solution is produced, great care should be taken to ensure the identicality of the new lot with the old. For precise, reproducible and intercomparable determination of P³² oligomer duplex molecule population t.5d values, each new lot should be compared to a gold standard lot (i.e., a high quality reference lot), and the previous lot.

(h) The dilution of the hybridized P³² duplex sample into the DK solution should also be large enough so that during the duration of the DK step, the P³² oligomer single strand molecules cannot significantly re-hybridize during the DK step. This dilution extent can be determine empirically. Alternatively, as discussed earlier an amount of unlabeled analyzed oligomer can be added to the DK solution so that no significant hybridization can occur between a single strand P³² oligomer molecule and a complement molecule during the DK step or the subsequent HA analysis.

(i) The DK solution containing the P³² oligomer duplex molecules is then put in a temperature incubator, and equilibrated to the desired DK analysis temperature, as quickly as possible. The rapidity with which the DK solution must be equilibrated to the desired DK analysis temperature depends on the desired time of DK analysis incubation before the first DK analysis data point is taken. It is preferred that the equilibration time be one tenth or less of the first time point duration. The equilibration time should be measured empirically.

(j) The analyzed DK analysis sample solution containing the P³² oligomer duplexes can be: In the form of a single incubated solution, from which an aliquot is removed at each desired time point; or in the form of identical multiple separate incubated DK analysis sample solutions, which have the same zero time of incubation in the same incubator, and one or more separate samples are removed from the incubator at each time point; or in the form of identical multiple separate DK analysis sample solutions which have different zero times of incubation and a different sample is removed from the incubator at the same or different desired incubation time. The method used for the DK analysis data reported herein, is the second described method, and for simplicity this method will be emphasized. Each of the other methods is a viable method, and each has advantages and disadvantages which will be apparent to one of skill in the art.

(k) For the DK analysis sample solution incubation method used here, it is desirable to use a circulating water temperature bath in order to ensure that all of the separate samples are exposed to the same temperature. In addition, it is highly desirable that the circulating temperature bath have excellent temperature stability and control characteristics at the desired incubation temperature. For the P³² oligomer duplex DK analysis reported herein, the closed circulating water bath used as an incubator, had a ±0.01° C. temperature stability.

(l) For each desired time point the corrected hybridization extent or % duplex remaining, is determined as described earlier.

(m) The corrected hybridization extent for each time point is then normalized to the DK analysis FD value. Here (the normalized corrected hybridization extent value)=(the corrected hybridization extent value)÷(the FD value). This normalized value represents the fraction of the starting P³² oligomer duplex molecules which are not dissociated at time t=x of incubation.

(n) The dissociation kinetic data points are then graphed by plotting (the log of the value for the fraction of P³² oligomer duplex molecules not dissociated) versus (the time of incubation t). Herein for simplicity, the fraction of P³² oligomer duplex molecules not dissociated is termed the fraction of duplex left or the % of duplex left, and the DK analysis results for any particular P³² oligomer duplex analysis results are plotted in terms of (log percent duplex left) vs (time t in minutes). When possible the t.5d value for a P³² oligomer duplex population is determined as described earlier.

(o) The P³² oligomer duplex dissociation kinetic profile represented on the graph is then analyzed. The DK analysis results can also be analyzed without graphing, as will be apparent to one of skill in the art. One of skill in the art will also recognize that a variety of other non-HA methods exist in the prior art for the determination of the kinetics of complete strand separation of oligomer duplexes.

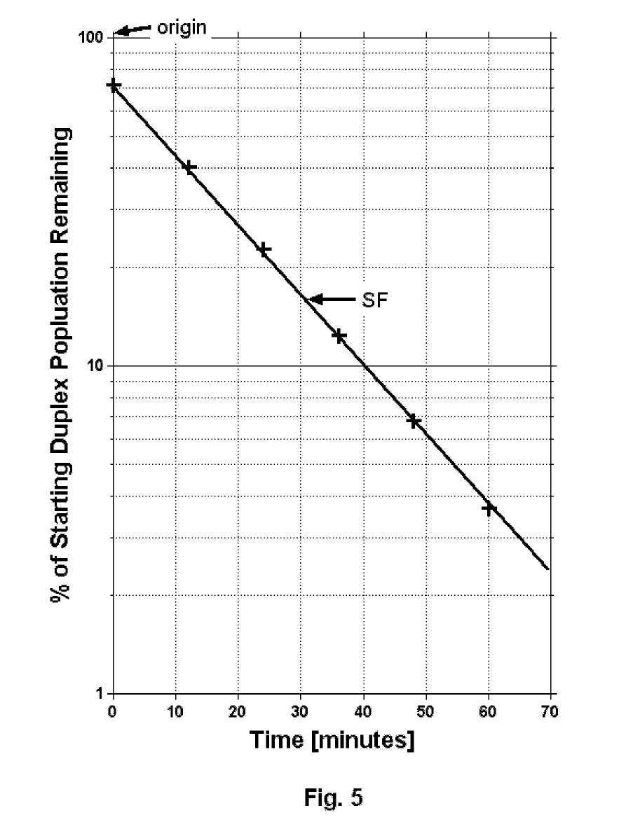

A P³² oligomer duplex molecule population DK profile was determined for hybridized P³² dA₃₅ oligomer·dT₃₅ oligomer duplexes in 0.09 MPB, 0.135 M total Na⁺, 0.01M LLS, pH6.8, DK analysis solution at 50° C. This DK profile is presented in FIG. 5. Herein, the said DK analysis solution is termed the Homopolymer Dissociation Buffer, or HDB. Both the dT₃₅ and dA₃₅ oligomers represent the purified N oligomer fraction. The hybridization reaction was done in HDB by placing the sealed vial containing the hybridization solution in a 62° C. circulating water bath and incubating at 62° C. for 10 m. The water bath was then turned off. The bath cooled slowly to room temperature overnight. The initial rate of cooling was 1° C. per 10 m in the first hour. Herein, this method of hybridization will be termed the annealing or stringent hybridization method. The HDB hybridization solution had a volume of 54 microliters and contained 7.9 picomoles of unlabeled dT₃₅ oligomer and 1.9 picomoles (Pm) of P³² dA₃₅ oligomer. The FD value for this analysis was 98.7%.

For the DK analysis a 5 microliter aliquot of the hybridization solution was diluted into 50 ml of HDB. The final concentration of unlabeled dT35 in the HDB is 1.5×10-11M. At this low dT35 oligomer concentration and the 50° C. DK analysis temperature, no significant hybridization can occur between dissociated single strand P³² dA₃₅ oligomer and the unlabeled dT₃₅ oligomer during the analysis. As an indication of this the slow portion of the DK profile is essentially linear below 10% duplex left.

As discussed earlier, when the measured DK profile for a homogeneous population of oligomer duplex molecules is presented in a graph which plots (the log of the percent oligomer duplex molecules left) vs (t), the DK profile has the following characteristics. (a) The measured DK data points will all fall on the same straight line. (b) The straight line formed by the DK data points will pass through the origin of the graph, that is the 100% duplex left, t=0, point on the graph. If an oligomer duplex population DK profile does not have both these properties, then the analyzed population of oligomer duplexes is not a homogeneous population of oligomer duplex molecules, and different oligomer duplex molecules in the analyzed oligomer duplex population have different DK kinetics and different physical chemical properties.

The oligomer dA₃₅·dT₃₅ duplex molecule population analyzed in FIG. 5 produces a DK profile which has the following characteristics. (a) The measured DK data points all fall on a straight line. (b) The straight line formed by the DK data points does not pass through the origin of the graph, but passes through a % duplex left value of about 72%. Therefore, the DK profile of Table 5 directly indicates that the analyses P³² dA₃₅·dT₃₅ duplex molecule population is heterogeneous and composed of at least two P³² oligomer duplex fractions. One fraction is represented by the straight line portion of the DK profile. The P³² dA₃₅ oligomer duplex molecule population represented by the straight line has much slower dissociation kinetics than the other fraction. Herein, this P³² dA₃₅ oligomer duplex molecule population is termed the slow fraction, or SF. The SF has a t.5d=14.2 m. The SF straight line intercepts the ordinate at a % duplex left value of 72% at zero time of dissociation. Thus, the SF represents 72% of the total P³² dA₃₅ oligomer molecule duplex population which was analyzed. Note that here, the FD=FH for the P³² dA₃₅ oligomer prep. The rapidly dissociating P³² dA₃₅ oligomer duplex molecule fraction consists of (100%−72%) or 28% of the analyzed P³² dA₃₅ oligomer duplex molecules. Most, if not essentially all of this 28% P³² dA₃₅ oligomer duplex molecule population dissociates much faster than does the SF. Herein, this 28% fraction is termed the fast fraction or FF. As indicated by the straight line on Table 5, the dissociation of the FF is complete by the time the first DK time point is measured.

Note that denaturing PAGE analysis indicated that the analyzed P³² dA₃₅ oligomer had a nucleotide length of N=35 before the DK analysis, and that after the DK analysis the FF and SF P³² dA₃₅ oligomer molecules also had a nucleotide length of N=35. There was no indication of an N−1 or N+1 fraction in any of the analyzed P³² dA₃₅ oligomer molecule populations as measured by denaturing PAGE analysis under conditions where the N−1 was known to be detectable.

Note that under the HDB 50° C. DK analysis conditions (dT₃₅)₂·(P³² dA₃₅) triplex molecules are known to be unstable. In addition, the annealing process is known to produce dA₃₅·dT₃₅ oligomer duplexes in which all 35 bases in one strand are base paired with a base in the complementary strand. This will be discussed below.

Similar DK analysis profiles were obtained for each of over 60 separately synthesized and purified N oligomer preparations. These stringent P³² oligomer duplex DK analysis results are summarized in Table 3. In Table 3, oligomers 1-33 represent homopolymer sequences. Oligomer (1) represents a dA₁₅ oligomer, while oligomers (2)-(4) represent dA₃₄ or dT₃₄ oligomers. All but one of the oligomers (5)-(33) represents either dA₃₅ or dT₃₅ N oligomer preps, while the exception represents the oligomer dC₃₅. Oligomers (34)-(63) represent different virus, prokaryote and eukaryote biological nucleotide sequences. Only six of the 63 different purified N oligomer preparations were associated with SF values of 90% or more.

TABLE 3
DK Analysis of a Variety of Stringent P³² Oligomer Duplex Preparations

| | | | Oligomer | DK Analysis | |
|---|---|---|---|---|---|
| | Purified N | | Nucleotide | Measured % Duplex^{(c)} | |
| | Oligomer Type^{(a)} | | Length | In | |
| | HS | BS | (N) | FF^{(b)} | SF |
| (1) | x | | 15 | 12 | 88 |
| (2) | x | | 34 | 25 | 75 |
| (3) | x | | 34 | 35 | 65 |
| (4) | x | | 34 | 22 | 78 |
| (5) | x | | 35 | 20 | 80 |
| (6) | x | | 35 | 30 | 70 |
| (7) | x | | 35 | 35 | 65 |
| (8) | x | | 35 | 35 | 65 |
| (9) | x | | 35 | 30 | 70 |
| (10) | x | | 35 | 30 | 70 |
| (11) | x | | 35 | 20 | 80 |
| (12) | x | | 35 | 10 | 90 |
| (13) | x | | 35 | 15 | 85 |
| (14) | x | | 35 | 18 | 82 |
| (15) | x | | 35 | 35 | 65 |
| (16) | x | | 35 | 28 | 72 |
| (17) | x | | 35 | 24 | 76 |
| (18) | x | | 35 | 28 | 72 |
| (19) | x | | 35 | 19 | 81 |
| (20) | x | | 35 | 22 | 78 |
| (21) | x | | 35 | 73 | 27 |
| (22) | x | | 35 | 89 | 11 |
| (23) | x | | 35 | 50 | 50 |
| (24) | x | | 35 | 20 | 80 |
| (25) | x | | 35 | 42 | 58 |
| (26) | x | | 35 | 18 | 82 |
| (27) | x | | 35 | 7 | 93 |
| (28) | x | | 35 | 7 | 93 |
| (29) | x | | 35 | 5 | 95 |
| (30) | x | | 35 | 22 | 78 |
| (31) | x | | 35 | 8 | 92 |
| (32) | x | | 35 | 34 | 66 |
| (33) | x | | 35 | 40 | 60 |
| (34) | | x | 22 | 20 | 80 |
| (35) | | x | 24 | 20 | 80 |
| (36) | | x | 24 | 10 | 90 |
| (37) | | x | 24 | 25 | 75 |
| (38) | | x | 24 | 20 | 80 |
| (39) | x | 24 | 20 | 80 | |
| (40) | x | 24 | 20 | 80 | |
| (41) | x | 24 | 20 | 80 | |
| (42) | x | 29 | 40 | 60 | |
| (43) | x | 29 | 20 | 80 | |
| (44) | x | 29 | 25 | 75 | |
| (45) | x | 29 | 48 | 52 | |
| (46) | x | 29 | 44 | 56 | |
| (47) | x | 29 | 15 | 85 | |
| (48) | x | 47 | 30 | 70 | |
| (49) | x | 47 | 30 | 70 | |
| (50) | x | 47 | 67 | 33 | |
| (51) | x | 47 | 34 | 66 | |
| (52) | x | 47 | 38 | 62 | |
| (53) | x | 47 | 49 | 51 | |
| (54) | x | 50 | 51 | 49 | |
| (55) | x | 50 | >98 | <2 | |
| (56) | x | 55 | 33 | 67 | |
| (57) | x | 55 | 57 | 43 | |
| (58) | x | 60 | 50 | 50 | |
| (59) | x | 64 | 65 | 35 | |
| (60) | x | 67 | 40 | 60 | |
| (61) | x | 89 | 79 | 21 | |
| (62) | x | 89 | 66 | 34 | |
| (63) | x | 89 | >90 | <10 | |

^{(a)}Each number represents a separately synthesized oligomer. Each oligomer was purified for the intended N oligomer fraction. Each different analysis represents a DK analysis of the purified N oligomer fraction. HS = Homopolymer Sequence BS = Biological Sequence
^{(b)}Value for FF represents the percent of total duplex identified as FF.
^{(c)}For all P³² analysed oligomer duplexes the P³² oligomer and unlabeled complementary oligomer molecules have the same N value.

Only one of these, the N=24 oligomer (36) represents a biological sequence. All of the other SF>90% oligomers were either dA₃₅ or dT₃₅ oligomers. Each of these oligomers had been subjected to extra purification effort. Other dT₃₅ and dA₃₅ oligomers were represented by the producers to have undergone similar extra purification procedures but had significantly lower SF values. The purified N oligomers were produced by multiple sources, which included three diagnostic companies, ten commercial oligomer producers, and several research labs and other commercial sources which produce oligos for internal use and for use in an oligomer application product.

For a particular stringently hybridized P³² oligomer duplex preparation. The measured % SF value is essentially the same for replicate determinations done in the same day or different days. For a particular N oligomer preparation, separately produced stringent P³² oligomer duplexes have essentially the same % SF values, even when different aliquots of the complementary oligomer are labeled and used to produce the stringent P³² oligomer duplexes. For a particular oligomer lot, separately produced P³² oligomer preparations will produce stringent P³² oligomer duplexes which have essentially the same % SF values. Further, stringently produced P³² oligomer duplexes which are DK analyzed at different temperatures have essentially the same % SF value.

For a particular P³² oligomer duplex preparation which is produced under non-stringent hybridization conditions, the measured % SF value may not be the same for replicate determinations done on the same or different days. Herein, a P³² oligomer duplex prep which is produced under non-stringent hybridization conditions is termed a non-stringent P³² oligomer duplex prep.

The measured % SF value for a particular P³² oligomer is reproducible when the stringent annealing process is used to produce the analyzed P³² oligomer duplexes. The following experimental results illustrate this. (a) Prepare and characterize a P³² dT₃₅ oligomer prep. Said P³² dT₃₅ oligomer has a measured FH value of 99.2% and a ZT value of 0.9% in the 0.09M P, 0.01% LLS (0.135M Na⁺) HA analysis and DK analysis solution. Herein, this solution has been termed the Homopolymer Dissociation Buffer, or HDB. (b) The P³² dT₃₅ oligomer is mixed with an excess of unlabeled dA₃₅ oligomer in HDB. The unlabeled dA₃₅ concentration in the 0.106 ml HDB solution is 2.9×10⁻⁸M. (c) The sealed hybridization mixture is annealed as described earlier, starting at 62° C. and incubating overnight. Herein, such annealed P³² oligomer duplexes are termed stringent duplexes. (d) An aliquot (5 microliters) of the annealing mixture was then diluted into 50 ml of 25° C. HDB and the FH and FD determined. Here HDB serves as both the HA analysis separation solution and the hybridization solution. In this dilution the dA₃₅ oligomer is at a concentration of 2.9×10⁻¹³M. The measured FH and FD value for this dilution is 99.2%. (e) Multiple separate 4 ml aliquots of the dilution were then placed in sealed tubes for DK analysis. To each tube was added 4.5 Pm of unlabeled dT₃₅ oligomer to prevent re-hybridization of dissociated P³² dT₃₅ during the DK analysis procedure. The final unlabeled dT₃₅ oligomer concentration is about 10⁻⁸M. (f) Each aliquot is then heated at 50° C. for a specified time and analyzed as discussed earlier to determine the normalized % P³² dT₃₅ duplex remaining value for each time point. (g) The DK data points are then plotted as described earlier to produce the DK profile for the P³² dT₃₅ oligomer duplex molecule preparation. (h) The % SF value and t.5d value is then determined from the DK profile. For these stringent P³² dT₃₅ oligomer duplexes the % SF value equaled 80%±roughly 2% on the same day the stringent P³² dT₃₅ duplexes were first diluted into the HDB solution. The measured 50° C. HDB t.5d value for these stringent P³² dT₃₅·dA₃₅ oligomer duplexes was 11.8 m.

The above-described stringent P³² dT₃₅ oligomer duplex DK analysis was repeated one day later using the same preparations of P³² dT₃₅ oligomer and dA₃₅ oligomer which had been pretreated at 42° C. for 25 m with 0.1M NaOH. Upon annealing and DK analysis of the base treated P³² dT₃₅ duplexes the FH and FD value was 99.1%, and the measured value for the % SF was about 82%. For these stringent P³² dT₃₅·dA₃₅ duplexes the 50° C. HDB t.5d value was 11 m.

The above-described first stringent P³² dT₃₅ oligomer duplex DK analysis was repeated two days later using the same P³² dT₃₅ oligomer and dA₃₅ oligomer preps used originally. The base treatment was not done. At the same time, the same P³² dT₃₅ oligomer was hybridized to unlabeled dA₃₄ oligomer and stringent P³² dT₃₅·dA₃₄ duplexes were produced and analyzed as described above. Here the stringent P³² dT₃₅·dA₃₅ duplexes had a % SF value of about 80% while the stringent P³² dT₃₅·dA₃₄ duplexes also had a % SF value of about 80%. The measured t.5d value was 12 m for the P³² dT₃₅·dA₃₅ duplexes and 9 m for the P³² dT₃₅·dT₃₄ duplexes.

Similar results have been obtained for stringently produced P³² dA₃₅·dT₃₅ duplexes as well as stringently produced biological sequence P³² oligomer duplexes. Overall the reproducibility of measured % SF values for stringently produced P³² oligomer duplexes is much better than the reproducibility of the t.5d values for the same stringently produced P³² oligomer duplexes. As discussed earlier the t.5d values are very sensitive to differences in temperature and salt concentration. As discussed, significant differences in t.5d can be associated with different preparations of HDB.

The effect of a non-stringent hybridization process for producing P³² dT₃₅·dA₃₅ duplexes is illustrated by the following. (a) Use the same P³² dT₃₅ oligomer prep and unlabeled dA₃₅ oligomer prep as used for the above-described illustrations, to produce a similar hybridization mixture in HDB. (b) Instead of annealing the hybridization mixture, simply incubate at 25° C. to hybridize. The excess dA₃₅ oligomer molar concentration for the hybridization mixture is about 1.1×10⁻⁷M in the HDB. Under the 25° C., HDB conditions the kₐ for the P³² dT₃₅+excess dA₃₅ is about 4×10⁶ M⁻¹ sec⁻¹, and the time for one-half hybridization is about 3 seconds. (c) At 20 m of hybridization a small aliquot of the hybridization mixture is diluted into 50 ml of 25° C. HDB. The remaining hybridization stock mixture remained at 25° C. (d) The measured FH and FD value is 96% and the ZT value is 1.3%. (e) DK analysis is done on the dilution as described above and the % SF value is measured. (f) Steps c-e are repeated for the hybridization mixture stock at 3 hours of incubation at 25° C., at 23.2 hours and 76 hours of incubation at 25° C. (g) The measured % SF and t.5d values observed for each time point are presented in Table 4 for these non-stringently hybridized P³² dT₃₅·dA₃₅ duplexes. These results indicate that at 20 m of hybridization the % SF value is only 50% for the early non-stringent P³² dT₃₅·dA₃₅ duplexes, relative to the stringent % SF value for the P³² dT₃₅·dA₃₅ duplexes of 80%. Over 72 hours the non-stringent P³² dT₃₅·dA₃₅ duplex % SF value increases to 70%. It appears that over time the non-stringent P³² dT₃₅·dA₃₅ duplexes incubated at 25° C. rearrange to form higher quality duplexes. With enough time, it is likely that the non-stringent hybridized P³² dT35·dA₃₅ duplex preparation would have a % SF value of 80%.

TABLE 4
% SF and t.5d Values for Non-Stringently Hybridized P32 dT₃₅.dA₃₅
Duplexes

| | | 50° C. |
|---|---|---|
| 25° C. Hybridization | % | HDB^{(a)} |
| Time (Hours) | SF | t.5d (m) |
| 0.33 h | 49% | 15 m |
| 3 h | 61% | 14.8 m |
| 23.2 h | 68% | 15 m |
| 76 h | 70% | 15.5 m |

^{(a)}In a different HDB preparation the measured t.5d value for P32 dT₃₅.dA₃₅ duplexes produced with the same P³² dT₃₅ oligomer prep used here, was about 11-12 m.

Note that although the % SF value for the non-stringent P³² dT₃₅·dA₃₅ duplexes significantly increases with time, the t.5d values are about the same.

For a particular stringent or non-stringent P³² oligomer duplex preparation, the t.5d value of the SF is essentially the same for replicate determinations on the same and different days, when the temperature and DK analysis solution composition are essentially the same for each replicate. Similarly, for particular stringent or non-stringent P³² oligomer duplex preparations which are separately produced from the same P³² oligomer prep, or different P³² oligomer preps from the same oligomer lot, or from different P³² preps from different lots of the same oligomer, the t.5d value of the SF fraction is essentially the same when the DK analysis temperature and DK analysis solution composition are essentially the same for each compared DK analysis. As discussed, the SF fraction t.5d value is very sensitive to differences in the temperature of DK analysis and in the cation composition of the DK analysis solution used. This is illustrated in Tables 5 and 6.

TABLE 5
Effect of Salt Concentration on the Measured t.5d Value for a Particular Oligomer Duplex

| P³² Oligomer Duplex | | | | ^{(c)}DK Analysis | Measured P³² | | |
|---|---|---|---|---|---|---|---|
| Nucleotide Length (N) | | | ^{(b)}DK Analysis | Solution Cation | Duplex t.5d | Fold Change In | |
| and Type | | | Temperature (° C.) | Concentration | (m) | Cation Conc. | t.5d |
| (1) | N = 24 | DNA | 58° C. | 0.135 M Na⁺ | 83 m | 1.12 | 1.6 |
| | ^{(a)}BS 1 | | 58° C. | 0.12 M Na⁺ | 53 m | | |
| | PM | | 60° C. | 0.135 M Na⁺ | 16 m | 10 | 2.1 |
| | | | 60° C. | 0.01 M Mg⁺⁺ | 67 m | | |
| | | | 60° C. | 0.001 M Mg¹ | 32 m | | |
| (2) | N = 24 | DNA | 53° C. | 0.135 M Na⁺ | 43 m | 1.12 | 1.6 |
| | BS 2 | | 53° C. | 0.12 M Na⁺ | 27 m | | |
| | MM | | 60° C. | 1 M Na⁺ | 61 m | 2.04 | 3.3 |
| | | | 60° C. | 0.49 M Na⁺ | 18 m | | |
| (3) | N = 29 | DNA | 55° C. | 0.143 M Na⁺ | 22 m | 1 | 1 |
| | BS 3 | | 55° C. | 0.143 M Na⁺ | 61 m | 1.14 | 2.8 |
| | PM | | | 0.02 M Li⁺ | | | |
| | | | 55° C. | 0.143 M Na⁺ | 185 m | 1.34 | 8.4 |
| | | | | 0.05 M Li⁺ | | | |

^{(a)}Biological Sequence = BS; PM = Perfect Match; MM = Mismatched
^{(b)}Circulating water bath had a temperature stability of ±0.01° C.
^{(c)}DK analysis solution pH ≈ 6.8.

Table 5 presents the effect of differences in the DK analysis solution cation concentration on the P³² oligomer duplex t.5d value. It is clear that small differences in the DK dissociation solution cause significant differences in the measured t.5d value for a P³² oligomer duplex. Cation concentration differences as small as 10 to 15 percent can result in a 1.6 fold difference in the P³² oligomer duplex t.5d value at lower cation concentrations.

Table 6 presents the effect of differences in temperature on the measured t.5d value for P³² oligomer duplexes. The temperature effect on the t.5d value is quantitated in Table 6 in terms of the fold change in the t.5d value caused by a one degree change in temperature, at a constant salt concentration. The t.5d fold change is equal to, (the measured t.5d value at a lower temperature)÷(the measured t.5d value at the compared higher temperature). The t.5d fold change per ° C. is herein termed the t.5d fold change/° C. or t.5d FC/° C. or just the FC/° C.

TABLE 6
Effect of DK Analysis Temperature on the Measured t.5d Value for a
Particular Oligomer Duplex

| | | | DK Analysis | DK Analysis | | | |
|---|---|---|---|---|---|---|---|
| P³² Oligomer Duplex Nucleotide | | | Solution Cation | Temperature | Measured P³² | | (Fold Change In t.5d) |
| Length (N) and Type^{(a)} | | | Concentration | (° C.) | Duplex t.5d (m) | | ° C. |
| (1) | N = 15 | PM DNA | 2.04 M Na⁻ | 30° C. | 14.6 | m | 1.7 |
| | HS | dA₁₅.dT₁₅ | 2.04 M Na⁻ | 31.7° C. | 5.9 | m | |
| (2) | N = 22 | RNA.DNA | 0.375 M Na⁺ | 45.9° C. | 90 | m | 2.07 (45.9° C.-48° C.) |
| | 2 OMe | PM | 0.375 M Na⁺ | 47° C. | 37.5 | m | |
| | BS 4 | | 0.375 M Na⁺ | 48° C. | 19.5 | m | |
| (3) | N = 24 | DNA | 0.135 M Na⁺ | 53° C. | 43 | m | 2.38 |
| | BS 2 | | 0.135 M Na⁺ | 55° C. | 7.6 | m | |
| | 1 MM equivalent | | 0.49 M Na⁻ | 58° C. | 97 | m | 2.32 |
| | due to linker | | 0.49 M Na⁻ | 60° C. | 18 | m | |
| | between nucleotide | | 1 M Na⁺ | 60° C. | 61 | m | 2.26 (60° C.-63° C.) |
| | phosphates 15 and | | | 62° C. | 12 | m | |
| | 16. | | | 63° C. | 5.3 | m | |
| (4A) | N = 24 | DNA | 0.135 M Na⁺ | 56° C. | 530 | m | 2.45 (56° C.-60° C.) |
| | BS 1 | PM | 0.135 M Na⁺ | 58° C. | 88 | m | |
| | | | 0.135 M Na⁺ | 60° C. | 15 | m | |
| | | | 1 M Na⁺ | 65° C. | 59 | m | 2.26 |
| | | | 1 M Na⁺ | 67° C. | 11.5 | m | |
| | | | 0.135 M Na⁺ | 58° C. | 87 | m | 2.33 |
| | | | 0.135 M Na⁺ | 60° C. | 16 | m | |
| (4B) | N = 24 | DNA | 0.135 M Na⁺ | 52° C. | 150 | m | 2.29 |
| | BS 1 | 1 MM | 0.135 M Na⁺ | 54.5° C. | 19 | m | |
| (4C) | N = 24 | DNA | 0.135 M Na⁺ | 54.7° C. | 140 | m | 2.32 |
| | BS-1 | | 0.135 M Na⁺ | 57° C. | 20 | m | |
| | 1 MM equivalent | | 0.135 M Na⁺ | 57° C. | 20 | m | |
| | due to linker | | 0.135 M Na⁺ | 57° C. | 19.8 | m | |
| | between | | 0.135 M Na⁺ | 57° C. | 20 | m | |
| | nucleotide | | 0.135 M Na⁺ | 57° C. | 21 | m | |
| | phosphates | | 0.135 M Na⁺ | 57° C. | 21 | m | |
| | 11 and 12. | | | | | | |
| (4D) | N = 24 | DNA | 0.135 M Na⁺ | 46.5° C. | 115 | m | 2.34 |
| | BS 1 | 2 MM | 0.135 M Na⁺ | 48.1° C. | 29.5 | m | |
| | | | 1 M Na⁺ | 53° C. | 130 | m | 2.24 |
| | | | | 55° C. | 26 | m | |
| (5) | N = 29 | Chimeric RNA | 0.135 M Na⁺ | 51° C. | 220 | m | 2.69 (51° C.-55° C.) |
| | BS 3 | and DNA | | 52° C. | 84 | m | |
| | | Oligomer | | 53° C. | 29 | m | |
| | | PM | | 54° C. | 11.4 | m | |
| | | | | 55° C. | 4.2 | m | |
| (6) | N = 35 | DNA | 0.15 M Na⁻ | 77° C. | 90 | m | 2.54 |
| | HS | PM | | 79° C. | 16.4 | m | |
| | dC₃₅.dG₃₅ | | | | | | |
| (7) | N = 34 | DNA | 0.135 M Na⁺ | 48° C. | 93 | m | 3.32 |
| | HS | PM | 0.135 M Na⁺ | 50° C. | 7.5 | m | |
| | dT₃₅.dA₃₄ | | | | | | |
| (8) | N = 35 | DNA | 0.135 M Na⁺ | 48° C. | 125 | m | 3.33 |
| | HS | PM | 0.135 M Na⁺ | 50° C. | 11.3 | m | |
| | dT₃₅.dA₃₅ | | 0.135 M Na⁺ | 48° C. | 145 | m | 3.38 |
| | | | 0.135 M Na⁺ | 50° C. | 12.7 | m | |
| (9) | N = 35 | DNA | 0.135 M Na⁺ | 45° C. | 125 | m | 2.9 |
| | HS 5 | 1 MM (A/G) | 0.135 M Na⁺ | 47° C. | 15.8 | m | |
| | (dT₃₄.A₁).dA₃₅ | | 0.135 M Na⁺ | 48° C. | 5.2 | m | |
| (10) | N = 35 | DNA | 0.135 M Na⁺ | 45° C. | 275 | m | 3.5 |
| | HS 5 | 1 MM | 0.135 M Na⁺ | 47° C. | 22.3 | m | |
| | (dT₃₄.G₁).dA₃₅ | | | | | | |
| (11) | N = 35 | DNA | 0.135 M Na⁺ | 45° C. | 72 | m | 3.36 |
| | 9 | | | | | | |
| | (dT₃₄.G₁).dA₃₅ | 1 MM | 0.135 M Na⁺ | 48° C. | 1.9 | m | |
| (12) | N = 35 | DNA | 0.135 M Na⁺ | 45° C. | 71 | m | 3.47 |
| | 18 | | | | | | |
| | (dT₃₄.G₁) | 1 MM | 0.135 M Na⁺ | 48° C. | 1.7 | m | |
| (13) | N = 35 | DNA | 0.135 M Na⁺ | 48° C. | 420 | m | 2.95 |
| | HS | PM | 0.135 M Na⁺ | 51° C. | 16.4 | m | |
| | dT₃₅.dA₃₀₀₋₅₀₀ | | | | | | |
| (14) | N = 47 | DNA | 0.165 M Na⁺ | 71.9° C. | 172 | m | 43 |
| | BS 5 | PM | | 73° C. | 32.2 | m | |
| | | | | 74° C. | 4 | | |
| (15) | N = 50 | DNA | 0.15 M Na⁻ | 67° C. | 168 | m | 42 |
| | BS 6 | PM | 0.15 M Na⁻ | 68.2° C. | 30 | m | |
| (16) | N = 55 | DNA | 0.15 M Na⁻ | 69.1° C. | 89 | m | 5.55 |
| | BS 7 | PM | 0.15 M Na⁻ | 70° C. | 19 | m | |
| | | | 0.15 M Na⁻ | 70.6° C. | 6.8 | m | |
| (17) | N = 67 | DNA | 0.165 M Na⁺ | 75° C. | 120 | m | 63 |
| | BS 9 | PM | 0.165 M Na⁺ | 76° C. | 19 | m | |
| (18) | N = 89 | DNA | 0.165 M Na⁺ | 84° C. | 290 | m | 16.8 |
| | BS 9 | PM | 0.165 M Na⁺ | 84.5° C. | 68 | m | |
| | | | | 85° C. | 17.3 | m | |
| (19) | P³² N = 21 BS.3′ | | 0.12 M Na⁻ | 52° C. | 74.2 | m | 2.04 |
| | Biotin + N = 51 | | 0.12 M Na⁻ | 54° C. | 17.8 | m | |
| | oligomer | | | | | | |
| | consisting of | | | | | | |
| | complementary BS | | | | | | |
| | N = 21 at 5′ end and | | | | | | |
| | dA₃₀.NH₂ at the 3′ | | | | | | |
| | end. | | | | | | |
| | BS Duplex is PM | | | | | | |
| (20) | As (19) except | | 0.12 M Na⁻ | 46° C. | 38.4 | m | 2.06 |
| | complementary BS | | 0.12 M Na⁻ | 48° C. | 9 | m | |
| | has a one base | | | | | | |
| | deletion at position | | | | | | |
| | #9. BS Duplex | | | | | | |
| | then has a bulge caused | | | | | | |
| | by an unpaired | | | | | | |
| | base. | | | | | | |

^{(a)}BS = Biological Sequences: HS = Homopolymer Sequence: PM = Perfectly Base Pair Matched Duplex MM = Imperfectly Base Pair Matched Duplex or Mismatched Duplex. 1 MM designates one base pair mismatch in duplex and 2 MM designates two base pair mismatches in duplex.
^{(b)}Circulating water bath had a temperature stability of ±0.01° C.
^{(c)}DK Analysis Solution pH = 6.8.

The t.5d FC/° C. is equal to, (the measured t.5d fold change observed for a temperature difference of X° C.)^{1/x}, where X is equal to the difference in temperature of DK analysis in ° C., or in other words the X root of the observed fold change value. More simply, the (FC/° C.)=(FC)^{1/x}. The largest oligomer duplex FC/° C. value found in the prior art literature was for an N=20 fluorescent labeled duplex, and the FC/° C. value measured in 1 M Na⁺ was about 1.66 FC/° C. Prior art FC/° C. values for N=19 perfect match and mismatch DNA oligomers measured in about 1M Na⁺ equal about 1.3 FC/PC. Other prior art reported FC/° C. values for shorter oligomers range from about 1.2 FC/° C. to 1.45 FC/° C. in salt concentrations ranging from 0.012 M Na⁺ to 1 M Na⁺. The lowest FC/° C. value reported in Table 6 is 1.7 FC/° C. for an N=15 oligomer. The FC/° C. values reported in Table 6 range from 1.7 for an N=15 oligomer duplex, to 16.8 for an N=89 oligomer duplex. These FC/° C. values increase with oligomer nucleotide length, and the increase is roughly proportional to the increase in N value for both biological and homopolymer sequences. Further, the presence of mismatched base pairs in the analyzed duplex does not significantly affect the FC/° C. value for the particular N value. The effect of the temperature on the t.5d value is large even for short oligomer duplexes. Table 6 (3)-(4D) presents the FC/° C. values for two different biological sequence N=24 oligomer duplexes and mismatched versions of these duplexes. As is indicated in Table 6 the FC/° C. value for all of these N=24 oligomer duplexes is around 2.3 FC/° C. This occurs: (i) In various high and low cation concentrations; (ii) At temperatures ranging from 46.5° C. to 67° C.; (iii) For perfect match DNA oligomer and chimeric oligomer duplexes, (iv) For duplexes containing one or two mismatches or a linker which acts as a mismatch. Similar general characteristics have been observed for all of the oligomer duplexes analyzed. Table 6 provides ample evidence that the effect of the temperature differences on the t.5d of analyzed oligomer duplexes is very large, even at high salt concentrations, and is much larger than prior art reported oligomer FC/° C. values. This has practical as well as other implications. These will be discussed below.

From a practical point of view the large FC/° C. values associated with even N=24 oligomer duplexes requires the use of a temperature incubator which is very stable in order to: Minimize the variability in the DK analysis data points obtained for a single DK profile determination for one P³² oligomer duplex sample; and to minimize the variability between measured t.5d values for replicate DK analyses and compared DK analyses for different P³² oligomer preps. The circulating temperature bath used to produce the DK profiles discussed herein was a Lauda MS circulating water bath with a reported temperature setting resolution of ±0.1° C. and a temperature control stability of ±0.01° C. For this circulating water bath then, when the water bath is on and the desired temperature is reached, the temperature remains stable to ±0.01° C. However, the absolute temperature of the temperature equilibrated water bath may vary by ±0.1° C. since the temperature setting resolution is ±0.1° C. The circulating water bath used for all of the t.5d determinations herein was this Lauda MS water bath.

The effect of the large FC/° C. values on the precision of DK analysis measured t.5d values can be measured by doing replicate DK analysis t.5d determinations on the same P³² oligomer duplex sample. Precision is a measure of the reproducibility or repeatability of the measured P³² oligomer duplex t.5d values. For simplicity, the measurement of the t.5d values for P³²dT₃₅·dA₃₅ stringent oligomer duplexes in 0.135 M Na⁺ at 50° C., will be primarily discussed. Under these conditions, the t.5d value generally equals about 12 m. Before such a t.5d determination the temperature bath is turned on, set to 50° C., and then allowed to equilibrate to temperature. Once the bath is at temperature equilibrium, the bath temperature varies by ±0.01° C. over time. Therefore, for the different time points in a DK analysis, the maximum temperature difference between particular time points is ±0.01° C. The FC/° C. value for the P³²dT₃₅·dA₃₅ oligomer duplexes in 0.135 M Na⁺ is about 3.3 FC/° C. The maximum temperature difference related deviation between particular data points in the same assay is equal to (3.3)^{0.02} or ±1.024 fold. Thus, the maximum within assay data point variation due to a bath temperature difference within the assay, is about 2.4%.

By the same logic, absent turning on of the water bath and pre-equilibrating it, the maximum between replicate assay data point variation due to a bath temperature difference for the separate assays done at different times, is also about 2.4%. Note that for this first water bath use where the bath is turned on and equilibrated to temperature, the absolute temperature of the bath is known to ±0.1° C. In the event that the bath is turned off and re-equilibrated, the absolute temperature is again known to ±0.1° C. Further, for this second water bath use, the actual equilibration temperature may differ from the actual temperature of the first water bath use by as much as 0.2° C. For a replicate P³²dT₃₅·dA₃₅ duplex t.5d value measured with a re-equilibrated water bath, the DK analysis temperature may deviate from that of the first use DK analysis by 0.2° C. For such a situation, the maximum temperature difference related deviation between the P³²dT₃₅·dA₃₅ duplex first use t.5d value and the second use t.5d value, is equal to (3.3)^{0.2} or ±1.27 fold. Here, the maximum between assay P³²dT₃₅·dA₃₅ duplex t.5d value variation due to a between assay bath temperature difference is about 27%, and the measured t.5d values can range from about 10.6 m to 13.6 m. Standard practice is to turn the water bath off at the end of the day, and at times to re-equilibrate during one work day. Table 7 presents results concerning the reproducibility of P³² dT₃₅·dA₃₅ duplex t.5d values over time and multiple re-equilibrations. Table 7 also presents results obtained with two different preparations of 0.135 M Na⁺ dissociation kinetic solution.

TABLE 7
Reproducibility of Stringent P³²dT₃₅.dA₃₅ Oligomer Duplex DK Analysis
t.5d Values Measured At 50° C. in One Preparation of 0.135 M Na⁺ HDB

| HDB Solution | P³²dT₃₅ | Time Span of | DK Analysis Result | |
|---|---|---|---|---|
| Lot | Preparation | Measurements | Replicate | t.5d Value |
| A | A | First Day | 1a | 11.7 m |
| | | First Day | 1b | 11.7 m |
| | | First Day | 1c | 11.3 m |
| | | First Day | 1d | 11.3 m |
| | | Fourth Day | 4a | 12.3 m |
| | | Fourth Day | 4b | 12.3 m |
| | | Fifth Day | 5a | 12.4 m |
| | | Fifth Day | 5b | 12.4 m |
| | | Sixth Day | 6a | 11.8 m |
| | | Sixth Day | 6b | 11.8 m |
| | | Sixth Day | 6c | 12 m |
| | | Sixth Day | 6d | 12 m |
| B | B | Day 1 | 1 | 13.8 m |
| | | Day 2 | 2 | 14.2 m |
| | | Day 3 | 3a | 12 m |
| | | Day 3 | 3b | 15 m |
| | | Day 14 | 14a | 15 m |
| | | Day 14 | 14b | 15.5 m |
| | | Day 14 | 14c | 14.8 m |
| | | Day 14 | 14d | 14.8 m |
| | | Day 14 | 14e | 15 m |
| | | Day 17 | 17 | 15 m |

Note that the above described DK profile t.5d and % SF results for synthesized purified N oligomer preps, are improved physical chemical property and functional characteristics results, relative to prior art produced physical chemical property results or functional characteristics results. Therefore, the above described measurements of the DK profile t.5d and % SF and % FF values for the synthesized and purified N oligomer preps is a practice of the present invention.

Interpretation of Chemically Synthesized P³² Oligomer Duplex Preparation DK Analysis Results.

For a P³² oligomer duplex molecule prep DK analysis profile the % SF value provides a quantitative parameter for characterizing the homogeneity or the heterogeneity of the P³² oligomer duplex molecule prep analyzed. The interpretation of such a DK analysis % SF value is influenced by the following factors. (i) The relative nucleotide length or N value of the P³² oligomer and its unlabeled complementary oligomer. (ii) Whether the DK analyzed P³² oligomer duplex is stringent or non-stringent. (iii) The P³² oligomer FD and FH values. The effect of these factors on the interpretation of the % SF value for a P³² oligomer prep is discussed below.

First will be discussed a DK analysis situation where: The analyzed P³² oligomer analyzed and its unlabeled complementary oligomer are both chemically synthesized oligomers; the P³² oligomer prep FH and FD values are the same and equal 100%; the N values of the P³² oligomer and unlabeled complementary oligomer are the same and are intended to form a P³² oligomer duplex molecule which contains N base pairs; the P³² oligomer duplexes are stringent or non-stringent duplexes. Herein, the unlabeled complementary oligomer used is termed the UC oligomer.

For this first situation, when the % SF value is less than 100% for a stringent homopolymer P³² oligomer duplex prep, the P³² oligomer single strand molecule population can be known to be heterogeneous, and the magnitude of the % SF value is a quantitative measure of the degree of heterogeneity which exists in the P³² oligomer molecule population. Here, the fraction of the analyzed P³² oligomer molecule population, which is homogeneous, is equal to the (% SF÷100), while the fraction of the analyzed P³² oligomer molecule population, which is heterogeneous, is equal to 1−(% SF÷100).

For this first situation, when the % SF value is less than 100% for a non-stringent P³² oligomer duplex prep, it cannot be known whether the P³² oligomer single strand molecule population is homogeneous or heterogeneous. In addition, it cannot be known whether the UC oligomer single strand molecule population is homogeneous or heterogeneous. Here, it can be known that the P³² oligomer duplex molecule population is heterogeneous to 1−(% SF÷100) extent. Further, for biological sequence oligomers it can be known that one or both the P³² oligomer molecules and/or the UC oligomer molecules are heterogeneous. For homopolymer sequences however, it cannot be known whether the P³² oligomer and/or UC oligomer molecule populations are homogeneous or heterogeneous.

Second will be discussed a DK analysis situation which is identical to the first described stringent P³² oligomer duplex situation, except that the P³² oligomer FD value does not equal to the FH value. Here, it will be assumed that the P³² oligomer FH and FD values are 100% and 95% respectively. This means that 100% of the P³² oligomer molecules can be shown to be capable of hybridizing, but only 95% of these P³² oligomer molecules are detected as being hybridized under the DK analysis HA conditions.

For this second situation, when the % SF value is 100% for the DK analyzed stringent P³² oligomer duplex prep, the P³² oligomer duplex molecule population analyzed can be known to be homogeneous. However, the total P³² oligomer single strand molecule prep cannot be known to be homogeneous. This occurs because the P³² oligomer molecules associated with the P³² oligomer duplex molecules which are DK analyzed, represent only 95% of the total P³² oligomer molecules which are present in the total P³² oligomer molecule population. In this situation where the P³² oligomer FD value is less than its FH value, the FD value is lower because the conditions used for the DK analysis result in a fraction of the P³² oligomer single strands not being in a stable duplex form at the start of the DK analysis. As a consequence only a fraction, in this case 95%, of the total P³² oligomer molecules are in a duplex form and are DK analyzed. For a P³² oligomer prep, the FD value will be lower than the FH value when one or both of the following scenarios exist. (a) The P³² oligomer molecule population is heterogeneous and is composed of a homogeneous sub-population of P³² oligomer molecules and a heterogeneous sub-population of P³² oligomer molecules. Here, the heterogeneous sub-population of P³² oligomer molecules comprises 5% of the total molecules in the P³² oligomer prep.

Under non-stringent hybridization conditions, both the homogeneous and heterogeneous sub-populations of P³² oligomer molecules can stably hybridize to the UC oligomer. This results in a measured 100% FH value. However, under more stringent DK analysis conditions, the heterogeneous P³² oligomer molecule sub-population cannot form stable duplexes while the homogeneous P³² oligomer molecule sub-population can form stable duplexes. This results in a measured P³² oligomer prep FD value, which is less than the FH value. Here, the FD value is 95%. For this scenario, a P³² oligomer FD value which is significantly lower than the P³² oligomer FH value indicates that the P³² oligomer prep is heterogeneous. (b) The P³² oligomer molecule population is homogeneous, and under non-stringent hybridization conditions all of the P³² oligomer molecules can form stable duplexes. Here, the P³² oligomer FH value is 100%. However, the P³² oligomer duplex DK analysis is performed under more stringent conditions and under these more stringent DK analysis conditions the homogenous population of P³² oligomer molecules forms only a homogeneous population of P³² oligomer duplex molecules. In addition, under these more stringent DK analysis conditions, at the start of the DK analysis a fraction of the homogeneous P³² oligomer molecules is in a single strand state and a fraction of the homogeneous P³² oligomer molecules is in the duplex state. The more stringent DK analysis condition causes the dissociation of a fraction of the homogeneous P³² oligomer duplexes and creates an equilibrium state or an approach to equilibrium state for the P³² oligomer duplexes and single strand molecules. Here, a DK analysis will produce a 100% SF for the P³² oligomer duplexes analyzed and the P³² oligomer duplexes can be known to be homogeneous. For this scenario a P³² oligomer FD value which is significantly lower than the P³² oligomer FH value does not indicate that the P³² oligomer prep is heterogeneous. The above described (a) and (b) scenarios for a P³² oligomer can be distinguished by isolating the P³² oligomer molecules which are dissociated at the zero time of the DK analysis and doing a DK analysis on them under the same DK analysis conditions as used for the initial DK analysis. This will be discussed later.

Third will be discussed a DK analysis situation which is identical to the first described stringent P³² oligomer duplex situation, except that the P³² oligomer N value is shorter than the UC oligomer N value. Here, when the P³² oligomer DK analysis % SF value is 100%, then it can be known that the P³² oligomer molecule prep is homogeneous.

Fourth will be discussed a DK analysis situation which is identical to the first described stringent P³² oligomer duplex situation, except that the P³² oligomer molecule N value is larger than the UC oligomer N value. Here, when the P³² oligomer DK analysis % SF value is 100%, then it can be known that the P³² oligomer duplex molecules analyzed are homogeneous for their base paired portions. However, it cannot be known that the P³² oligomer single strand molecule population is homogeneous for the portion of the P³² oligomer molecule, which is associated with the P³² oligomer duplex molecule and is single stranded. This portion may or may not be heterogeneous. The homogeneity of this single strand portion of the P³² oligomer molecules can be determined by utilizing a UC oligomer of the same nucleotide length N as the P³² oligomer and which forms a P³² oligomer duplex which is N base pairs long.

Fifth will be discussed a DK analysis situation which is identical to the first described stringent P³² oligomer duplex situation, except that the P³² oligomer·UC oligomer duplex which is DK analyzed contains two single strand regions. Here, when the P³² oligomer duplex DK analysis % SF value equals 100%, then it can be known that the P³² oligomer duplex molecules analyzed are homogeneous in the base paired portion of the P³² oligomer duplexes. However, as discussed above in the fourth described situation, it cannot be known that the P³² oligomer single strand molecule population is homogeneous for the portion of the P³² oligomer molecule which is associated with the P³² oligomer duplex and is single stranded. This portion may or may not be homogeneous. The homogeneity of this single strand portion of the P³² oligomer can be determined by utilizing a UC oligomer of the same nucleotide length N as the P³² oligomer and which forms a P³² oligomer duplex which is N base pairs long.

It is clear from the above discussion that it is necessary to utilize stringently produced P³² oligomer duplexes in the DK analysis in order to be able to interpret the state of homogeneity of the P³² oligomer molecule preparations.

To this point the discussion on the interpretation of P³² oligomer duplex DK analysis % SF values have concerned the use of chemically synthesized UC oligomer and chemically synthesized P³² oligomer for the DK analysis. A conclusion of this discussion is that for such a DK analysis system, it is necessary to utilize stringently produced P³² oligomer duplexes in order to be able to clearly interpret the P³² oligomer duplex DK analysis % SF values obtained. Table 3 presents the stringent P³² oligomer duplex DK analysis % SF values for over 60 separate chemically synthesized P³² oligomer preps. None of these % SF values equaled 100% and all of them had values of 95% or lower, 57 had values below 90%, 41 had values below 80%, 27 below 70%, and 16 below 60%. Many of the Table 3% SF values represent studies where each complementary strand of a particular chemically synthesized oligomer duplex underwent P³² oligomer DK analysis. In all of these cases, both complementary oligomer preps were significantly heterogeneous. Thus, for the DK analysis of any particular chemically synthesized P³² oligomer duplex it is highly likely, if not essentially certain, that both the P³² oligomer prep and the UC oligomer prep are significantly heterogeneous. Further, it is also highly likely, but not necessarily certain, that both the P³² oligomer prep and the UC oligomer prep are comprised of a significant proportion of SF fraction oligomer molecules, which represent homogeneous oligomer molecule populations. Because of the presence of oligomer molecule homogeneity and heterogeneity in virtually all chemically synthesized oligomer preps it is necessary to utilize stringently produced P³² oligomer duplexes for the DK analysis, in order to be able to rationally interpret the meaning of a P³² oligomer DK analysis produced % SF value. Note that if the P³² oligomer prep or the UC oligomer prep used to produce the stringent P³² oligomer duplexes does not contain a SF related homogenous oligomer molecule population, no SF will be observed for the resulting P³² oligomer duplex DK analysis. Table 3 (56) appears to be such a situation.

Only a few of the P³² N oligomer preparations used to obtain the DK analysis results of Table 3 contained detectable N−1 P³² oligomer molecules. Further, the P³² oligomer preps which did contain detectable N−1 P³² oligomer, contained very small amounts. For such P³² oligomer preps the amount of N−1 P³² oligomer present was far less than the % FF observed for the stringent P³² oligomer duplexes produced using the P³² oligomer prep. Denaturing page analysis indicated a lower limit of detection of less than one part N−1 P³² dA₃₄ oligomer mixed with 19 parts of N P³² dA₃₅ oligomer. In this context Table 3 (15) represents the DK analysis of a stringent P³² dA₃₅-dT₃₅ duplex produced form a P³² dA₃₅ oligomer prep with an FD value equal to 99% and which contained no detectable N−1 P³² dA₃₄ oligomer. In addition, it was shown that the P³² dA₃₅ oligomer molecules are not damaged by the DK analysis process. The P³² dA oligomer molecules associated with the SF and the FF were shown to have an N=35 value both before and after the DK analysis, and were also shown to contain no detectable P³² dA₃₄ N−1 oligomer molecules. This Table 3 (15) P³² dA₃₅ oligomer prep was shown to contain about 35% FF and 65% SF. Table 3 (11) represents the DK analysis of a stringent P³² dT₃₅·dA₃₅ duplex produced from a P³² dT₃₅ oligomer prep with an FD value equal to 99%, and which contained no detectable N−1 P³² dT₃₄ oligomer molecules. In addition, it was shown that the P³² dT₃₅ oligomer molecules are not damaged by the DK analysis process. The P³² dT₃₅ oligomer molecules associated with the SF and the FF were shown to have an N=35 value both before and after the DK analysis, and were also shown to contain no detectable P³² dT₃₄ N−1 oligomer molecules. This Table 3 (15) P³² dT₃₅ oligomer prep contained about 20% FF and 80% SF.

Note that the effect of the DK analysis process on the intactness and integrity of the analyzed P³² oligomer molecules was checked many times. No effect of the DK analysis process on the intactness and/or integrity of the analyzed P³² oligomer molecules was detected. In addition, pretreatment of the P³² N oligomer prep with 0.1 NaOH for various time periods at 25° C. to 50° C. had essentially no effect on the P³² oligomer values for FH or FD, % SF, t.5d, or nucleotide length. This indicates that the chemically synthesized N oligomer molecules were completely deprotected during the purification process and that apurinic sites are also not detectable in the purified N oligomer preps. It was also demonstrated that exposure of the oligomer molecules to the radioactive P³² during and after labeling, did not detectably damage the oligomer molecules. Neither prolonged exposure during the P³² labeling nor prolonged storage of the P³² oligomer molecules resulted in detectable damage. Purified N oligomer preps representing a variety of biological nucleotide sequences have also been evaluated as described above, and these oligomers have essentially identical general characteristics as those described above for the homopolymer N oligomers.

As discussed earlier, prior art recognizes that prior art chemically synthesized oligomer preparations contain the intended N oligomer as well as a variety of N−X oligomers and has developed methods for obtaining oligomer preparations which are 95% or more pure for the intended N oligomer molecules. Prior art believes and represents that a small amount, 5% or so of N−X oligomers, often are present in the highly purified N oligomer. Prior art further believes that because of the purification process most of the N−X oligomers present are N−1 oligomers. Prior art believes that the homopolymer N−1 oligomer molecules in a purified N oligomer prep are identical to the N oligomer molecules, except for a deleted nucleotide which is present in the N oligomer and absent in the N−1 oligomer. As discussed earlier, prior art believes and practices the following concerning the chemically synthesized highly purified N oligomer preparations. (a) Oligomer molecules, which have the intended nucleotide length N, also have the intended nucleotide sequence. (b) Highly purified N oligomer molecules, which have the intended N value have essentially the same physical chemical properties. (c) Tacitly prior art then believes that in a purified N oligomer molecule preparation, all or essentially all N oligomer molecules, which are present, are essentially identical to one another and have essentially identical physical properties. (d) Tacitly then, prior art believes and practices that the highly purified N oligomer molecule populations in the purified N oligomer preps are essentially homogeneous populations of N oligomer molecules, except for the small fraction of N−1 oligomer which may be present in the purified oligomer prep. (e) Prior art further tacitly believes and practices that the homogeneous population of N oligomer molecules which are present in the purified N oligomer prep, also have the intended physical chemical properties.

The results summarized in Table 3 and the above discussion indicate that the above described prior art assumptions (c) and (d) are invalid for all 63 chemically synthesized purified N oligomer preps examined. These purified N oligomer preps were chemically synthesized and purified by a variety of commercial and research sources in the United States and Europe. Therefore, it is likely that the prior art assumptions (c) and (d) are generally, if not universally invalid for prior art oligomer synthesis and purification practice.

Prior art synthesized oligomers and purified N oligomer molecule preps are almost always designed and produced to have certain intended functional properties. The intended N value and nucleotide sequence and homogeneity determine the intended physical chemical properties of the oligomer prep, and the intended physical chemical properties of the oligomer prep determine the intended functional properties of the oligomer prep. Prior art chemical synthesis production of oligomer practice does not utilize methods for determining the following for prior art produced and purified N oligomer preps. (i) The actual functional properties of the purified oligomers. (ii) Whether the actual functional properties of the purified oligomer prep equal the intended functional properties for the oligomer. (iii) The actual homogeneity of the purified oligomers. (iv) Whether the actual homogeneity of the purified oligomers is the same as the intended oligomer homogeneity. (v) The actual values for the oligomers key functional characteristics such as the kₐ, FD, FH, % SF, and t.5d values. (vi) Whether the actual functional characteristic values are the same as the intended functional characteristic values. (vii) Whether the intended and/or actual oligomer functional characteristics are optimal for the intended oligomer application functional effectiveness. The discussion for this section is pertinent for items (i), (iii), (iv), and (v). Items (ii), (vi), and (vii) require the determination of the intended functional characteristics of the oligomer, and an evaluation as to whether the functional properties of the oligomer which are intrinsic to the intended physical chemical properties of the oligomer are desired functional properties. This issue will be discussed in a later section.

Note that for a stringent oligomer duplex DK profile, the SF is regarded as a homogeneous population of oligomer molecules, which represent the intended oligomer molecules.

Isolation and Characterization of P³² Oligomer Fractions Which are Enriched for SF P32 Oligomer or FF P32 Oligomer.

As discussed, stringent P32 oligomer FF duplexes dissociate much faster than stringent P32 oligomer SF duplexes in a DK analysis assay. In a typical DK analysis assay designed to determine the % SF of a P32 oligomer duplex prep, the P32 oligomer FF duplexes are essentially completely dissociated by the first time point. Generally, the average FF duplex dissociates hundreds of times faster than the SF duplexes. Because of this disparity in the FF and SF t.5d values, it is possible to readily fractionate stringent or non-stringent P32 oligomer duplexes into a FF, which is enriched for the P32 oligomer molecules associated with P32 oligomer FF duplexes in the total P32 oligomer duplex prep, and a SF which is enriched for the P32 oligomer molecules associated with the P32 oligomer SF duplexes in the total P32 oligomer duplex prep. The FF and SF can be recovered and evaluated.

This fractionation procedure utilizes the earlier described presently preferred HA method for separating hybridized from non-hybridized P³² oligomer. To illustrate this process the fractionation of a stringent P³² dT₃₅·dA₃₅ duplex molecule preparation is described following. (a) Prepare a stringent P³² dT₃₅·dA₃₅ duplex molecule preparation in HDB as described earlier. (b) Determine the FH, FD, % SF and t.5d values for the P³² dT₃₅ oligomer and the prepared stringent P³² dT₃₅·dA₃₅ oligomer duplex preparation under the chosen fractionation conditions, and under the standard 50° C. HDB P³² dT₃₅·dA₃₅ duplex DK analysis conditions. The fractionation conditions can vary according to the purpose of the fractionation. Here, the DK fractionation temperature condition used is 50° C. in HDB and the HA separation condition is 52° C. in HDB. For this P³² dT₃₅ oligomer prep the FH and FD values equaled 99.4%, and the 50° C., HDB DK analysis t.5d=15.8 m, and the 50° C. HDB % SF value was about 80%. (c) Dilute the stringent P³² dT₃₅·dA₃₅ duplex preparation into 4 ml of HDB and incubate the dilution at 50° C. for 15 m. (d) Then load the 4 ml aliquot onto the 52° C. HA column and incubate for 1 m, and then quickly pass the aliquot thru the HA column and then quickly wash the HA column with 4 ml of 52° C. HDB wash solution. This first 8 ml of HDB is collected and is termed the unbound fraction one or UF1. UF1 contains almost all of the P³² single strand oligomer, which was put on the column. (e) The HA column was then washed with about 40 ml of 52° C. HDB, which was collected in three scintillation vials. These fractions are termed UF2, UF3, and UF4. About 1-2% of the total P³² single strand oligomer is present in these fractions. (f) After UF4 slowly pass 4 ml of 0.3 M P through the HA column bed to recover the still hybridized P³² dT₃₅·dA₃₅ oligomer duplexes. This fraction was collected and is termed the bound fraction one or BF1. (g) The HA column bed is then dissolved in 6 M HCl and collected. This is termed the HCl fraction or HF. About 1% of the total (BF+HF) P³² CPM are present in the HF. (h) The P³² CPM present in each fraction collected was determined by Cerenkov counting in a scintillation counter. The use of such a CPM detection method allows the P³² dT₃₅ oligomer molecules to be used and re-analyzed again. (i) The UF1 sample contained about 42.3% of the total CPM analyzed. The P³² dT₃₅ oligomer present in UF1 is enriched for FF P³² dT₃₅ oligomer molecules. (j) The BF1 sample comprised 57% of the total input P³² CPM and is enriched for the SF P³² oligomer molecules. (k) The UF1 and BF1 P³² dT₃₅ oligomer molecules are saved and used for further analyses. Examples of such analyses are illustrated below. Note that after the fractionation procedure no degraded N−X P³² dT₃₅ oligomer molecules were detected in the isolated UF or BF P³² dT₃₅ oligomer fractions.

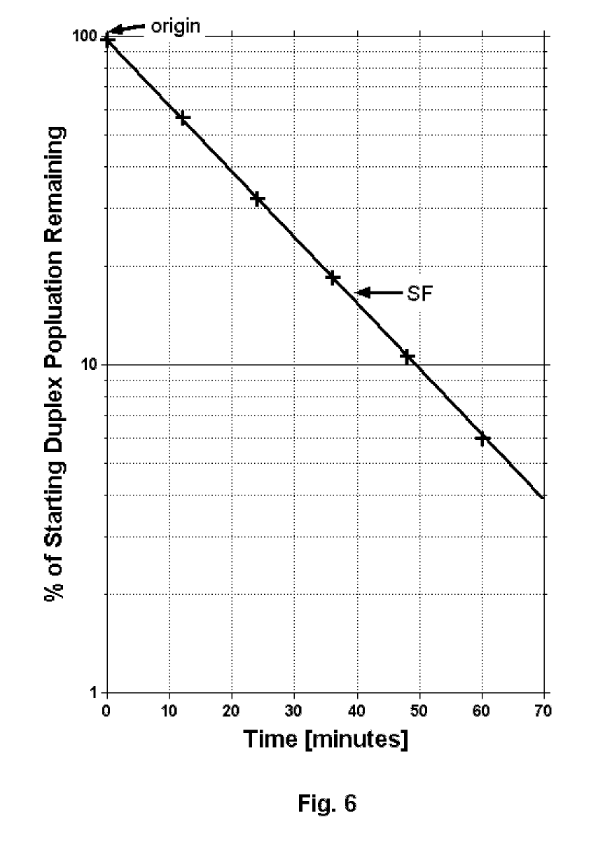

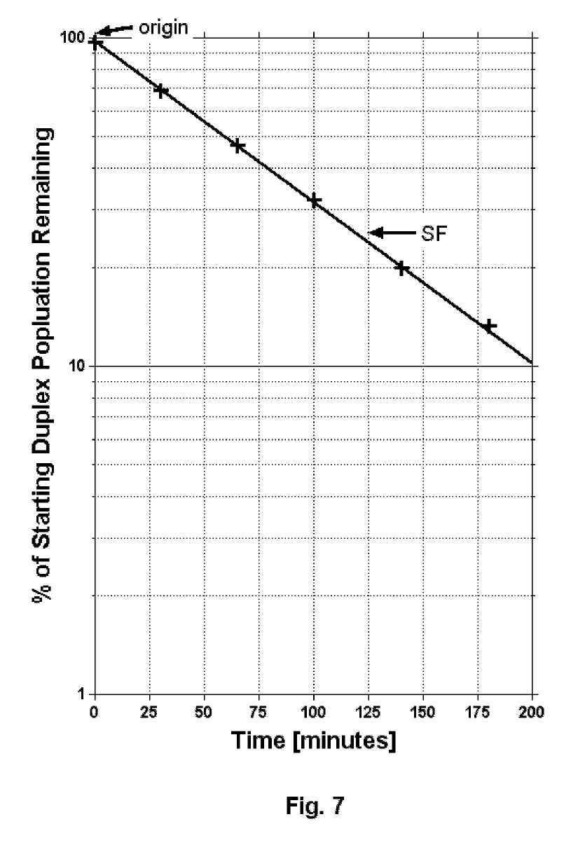

The fractionation procedure was designed to greatly enrich the BF fraction for SF P³² dT₃₅ oligomer molecules. The unfractionated stringent P³² dT₃₅·dA₃₅ duplex prep contained about 80% SF and 20% FF when analyzed at 50° C. in HDB, and the SF t.5d value was 15.8 m. The unfractionated stringent P³² dT₃₅ oligomer had an FH value of 99.4%. The isolated BF was checked in 50° C., HDB to determine how much of the isolated P³² dT₃₅ oligomer was hybridized and it was determined that 99.4% of the BF1 isolated P³² dT₃₅ oligomer molecules were in a hybridized form. The isolated BF stringent P³² dT₃₅·dA₃₅ duplexes were characterized by the earlier described DK analysis methods in HDB at both 50° C. and 48.8° C. The resulting DK profiles are presented in FIG. 6 (50° C.) and FIG. 7 (48.8° C.). Both BF DK profiles indicate that the BF is greatly enriched for the SF P³² dT₃₅·dA₃₅ oligomer duplexes. The unfractionated stringent P³² dT₃₅·dA₃₅ duplex prep contained about 80% SF P³² oligomer duplexes. The DK profiles of FIGS. 6 and 7 indicate a % SF value of around 97%. The 50° C. t.5d for the BF SF is about 15 m, while the 50° C. t.5d for the unfractionated stringent P³² dT₃₅·dA₃₅ duplex prep which was determined a week earlier was about 15.8 m. The 48.8° C. BF t.5d value equaled 62 m.

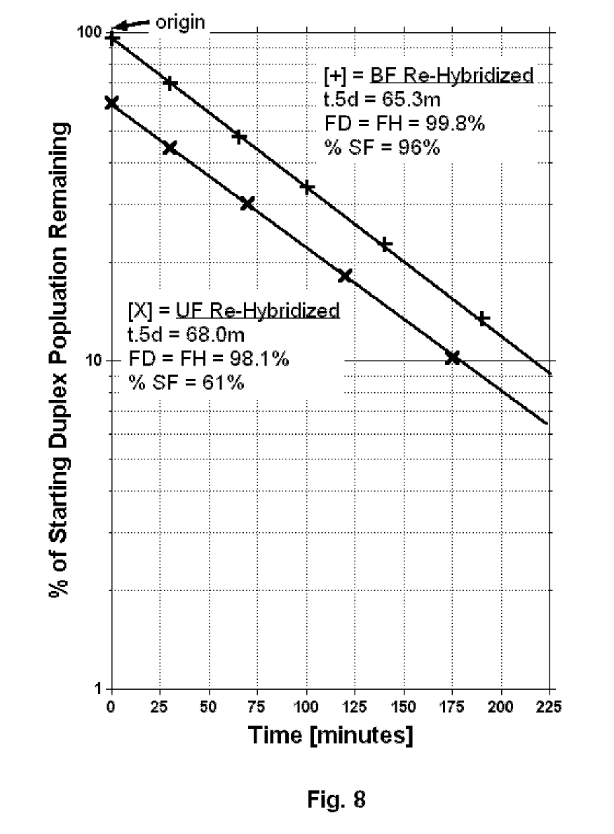

The 48.8° C. DK profile was done to slow the dissociation kinetics in order to obtain a better idea as to the relative dissociation kinetic difference of the SF and the FF. FIG. 8 presents the DK profiles for both the UF and BF stringent P³² dT₃₅·dA₃₅ duplexes at 48.8° C. in HDB. To produce these DK profiles excess UC dA₃₅ was added to the UF and BF and re-annealed to reform UF and BF stringent P³² dT₃₅·dA₃₅ oligomer duplexes. These duplexes were then DK analyzed as described earlier. The re-hybridized BF DK profile indicated an SF value of about 96% and a SF t.5d value of about 65 m. These values are quite similar to those of FIG. 7 for non-rehybridized BF duplexes. Note that the Table 8 BF DK profile demonstrates that the annealing method used works well to produce stringent oligomer duplexes of the highest quality. Note, further that the UC dA₃₅ oligomer prep used for the initial stringent hybridization annealing step and the re-hybridization annealing step was in large molar excess relative to the P³² dT₃₅ oligomer, and was known to contain 70% SF oligomer.

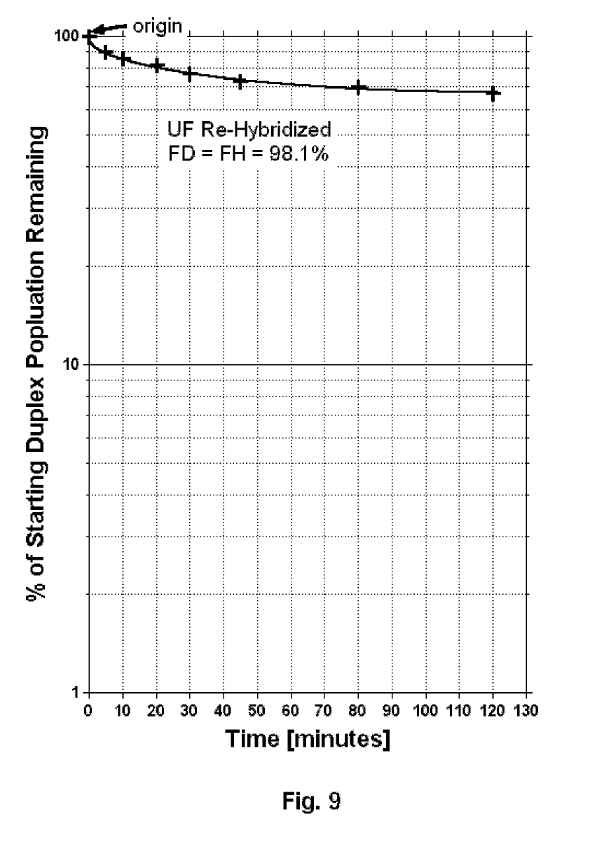

The re-hybridized DK profile of FIG. 8 indicates that the UF is enriched for the FF P³² dT₃₅ oligomer fraction. The unfractionated stringent P³² dT₃₅·dA₃₅ duplexes contained about 20% FF, while the UF contains about 39% FF. The 48.80 UF t.5d value for the SF is about 68 m, which is quite similar to the SF t.5d values of the BF. The UF DK profile of FIG. 8 also indicates clearly that the relative dissociation kinetics of the FF and SF are very different. In order to obtain a more detailed FF DK profile, a FF DK analysis was done at 45° C. in HDB, and this DK profile is presented in FIG. 9. Note that at 45° C. the t.5d of the SF is roughly 5500 m. Because of this, the duplex dissociation observed in FIG. 9 is essentially all due to the dissociation of the FF P³² dT₃₅·dA₃₅ oligomer duplexes.

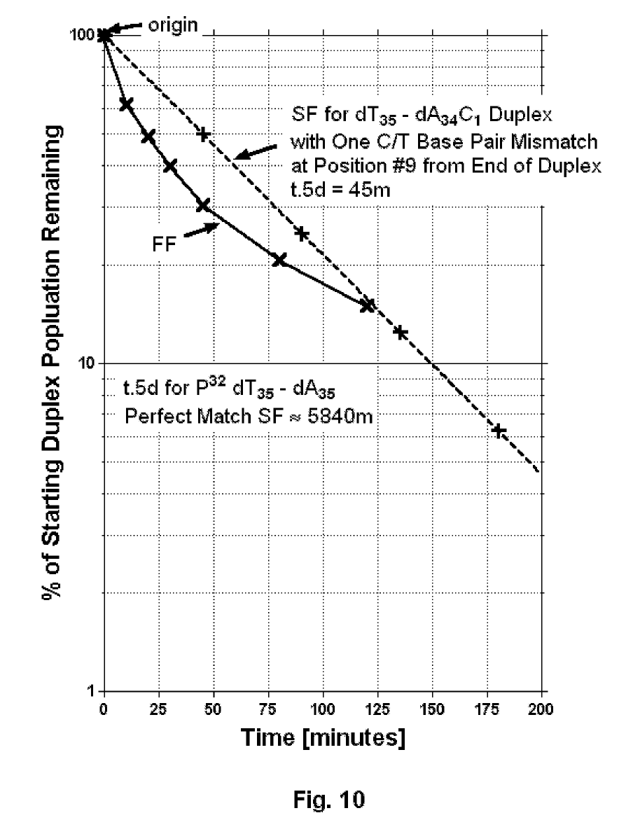

Further, this allows the use of the 45° C. UF DK profile data points to construct a normalized DK profile for just the FF P³² dT₃₅·dA₃₅ oligomer duplexes. This can be done because the total UF stringent duplex prep is known to contain 61% SF and 39% FF P³² dT₃₅ oligomer duplexes (see FIG. 8), and the % SF is the same at 45° C. as it is at 48.8° C. FIG. 10 presents this normalized DK profile for just the FF of the stringent UF P³² dT₃₅·dA₃₅ duplexes.

The curved nature of the re-hybridized FF stringent P³² dT₃₅·dA₃₅ duplex prep DK profile indicates that the FF stringent P³² dT₃₅·dA₃₅ duplex molecule population analyses is composed of P³² dT₃₅·dA₃₅ duplexes molecules which have different t.5d values. Therefore, the P³² dT₃₅ oligomer molecule population which is associated with the stringent FF P³² dT₃₅·dA₃₅ duplex molecule population analyzed, is a heterogeneous population of P³² dT₃₅ oligomer molecules which have detectably different physical chemical properties. Note again that no detectable N−X or N+X P³² oligomers were present in the isolated UF P³² dT₃₅ oligomer prep or in the isolated BF P³² dT₃₅ oligomer prep, or in the unfractionated P³² dT₃₅ oligomer prep. As a result, the unfractionated P³² dT₃₅ oligomer molecule population can be known to be composed of the following fractions. (a) An 80% fraction population (i.e., the SF) which is composed of P³² dT₃₅ oligomer molecules which have essentially the same physical chemical properties, and which presumably represent the intended N oligomer molecules. (b) A 20% fraction (i.e., the FF) which is composed of numerous sub-populations of P³² dT₃₅ oligomer molecules and the different sub-populations of P³² dT₃₅ oligomer molecules have different physical chemical properties. Because of (a) and (b) it can be concluded that the unfractionated P³² dT₃₅ molecule population is composed of a large homogeneous sub-population of P³² dT₃₅ oligomer molecules, and a smaller heterogeneous sub-population of P³² dT₃₅ oligomer molecules. It can be concluded then, that the unfractionated P³² dT₃₅ oligomer prep is heterogeneous, and that the chemically synthesized and N purified dT₃₅ oligomer preparation used to produce the P³² dT₃₅ prep is also heterogeneous. Further, it can be concluded that about one out of every five dT₃₅ molecules in the N purified oligomer prep is different.

The stringent P³² dT₃₅·dA₃₅ oligomer duplexes which make up the FF are clearly much less temperature stable than the stringent SF P³² dT₃₅·dA₃₅ oligomer duplexes. This indicates that the FF associated P³² dT₃₅ oligomers are damaged somehow relative to the SF associated dT₃₅ oligomer molecules. It is reasonable to assume that the basis for this damage is a chemical difference between the FF P³² dT₃₅ and SF P³² dT₃₅ oligomer molecules. The cause of such a chemical difference is not known. It has been established that the damage is not due to the following. (a) The presence of depurinated bases in the oligomer. (b) Incomplete deprotection of the oligomer. (c) A reduction in oligomer size or other damage caused by the analysis procedure, which includes exposure to high temperature for prolonged periods. (d) The presence of deleted bases in the N=35 P³² dT₃₅ FF oligomers. (e) Exposure to UV or fluorescent light during gel purification and other processes. (f) Radiation damage due to exposure to P³². Here, it was shown that allowing the labeled oligomer to incubate overnight in the small volume P³² labeling reaction had no effect on the oligomer characteristics, relative to a standard 40 m labeling period.

The above described basic method of isolating and characterization of oligomer preps has been applied to multiple dA₃₅ oligomer preps and multiple dT₃₅ oligomer preps as well as about 10 different biological sequence oligomer preps which had N values ranging from 22 to 89. In each case the stringent FF P³² oligomer duplexes dissociated very much faster than the stringent SF P³² oligomer duplexes, indicating that the P³² oligomer molecules associated with the FF are damaged relative to the P³² oligomer molecules associated with the SF. This indicates that the % FF values for all of the 63 stringent P³² oligomer duplex preps listed in Table 3 represent oligomer fractions which are damaged relative to the SF oligomer molecules. Since these 63 purified oligomer preps represent multiple commercial and research lab suppliers, and the oligomer synthesis and purification procedures are essentially state of the art for the prior art, it seems reasonable to conclude that the great majority, if not all, prior art purified N oligomer preparations are significantly heterogeneous, and contain significant quantities of “damaged” oligomer molecules which have an N value which is the same as the “undamaged” oligomer molecules in the oligomer preparation. As a result, the prior art practice and belief that for a purified oligomer preparation, the population of oligomer molecules which have the intended N value is a homogenous population of N oligomer molecules, is invalid for all or nearly all chemically synthesized and purified N oligomer preps.

Note that the above described isolation and characterization of oligomer fractions which are enriched for SF oligomer or FF oligomer, determines physical chemical property results and functional characteristics results which can be known to be improved relative to such prior art produced results. Therefore, the isolation and characterization of oligomer fractions, which are enriched for SF or FF oligomers, is a practice of the present invention.

Estimating the Extent of Damage Associated with the N Oligomer FF Molecules.

For this discussion the oligomer molecule fraction which is associated with the stringent P³² oligomer FF duplexes of a purified oligomer prep is termed the damaged oligomer fraction, and the oligomer molecules present in such a damaged fraction are termed damaged oligomers or damaged N oligomers. The damage associated with damaged oligomer molecules includes, but is not limited, to the following. (i) One or more chemically damaged or modified bases, sugars, or phosphates. (ii) One or more misincorporated bases, which causes a base pair mismatch. (iii) One or more deleted bases.

It is clear that the presence of such damaged oligomers will degrade the performance of the intended oligomer application. In order to properly evaluate the effect of the presence of such damaged oligomers on the intended use, it is necessary to have a measure of the fraction of the N oligomer prep, which consists of damaged oligomers, as well as a measure of the extent of damage associated with the damaged oligomer fraction. The stringent % FF value for a purified oligomer prep is a direct quantitative measure of the fraction of damaged oligomers in a purified N oligomer prep. A method for determining the % FF was described earlier in detail. The obvious heterogeneity of all damaged oligomer fractions examined thus far indicates that obtaining a precise extent of damage value for a damaged oligomer fraction is very complex, even for homopolymer oligomers. However, it is possible to fairly simply characterize the extent of damage in a relative semi-quantitative manner. The relative measure of oligomer damage used here involves the following. (a) Prepare a stringent P³² oligomer of interest mismatched duplex prep, which consists of the P³² oligomer of interest and a UC oligomer which contains one or more mismatched bases at a known position or positions in the oligomer nucleotide sequence. Then determine the DK profile for the mismatched stringent P³² oligomer duplex, and the t.5d value for the SF mismatched duplexes. Such a P³² oligomer mismatched SF duplex t.5d value is here termed a mismatched t.5d value, or a MM t.5d value. (b) Prepare a stringent P³² oligomer of interest perfect match duplex prep, which consists of the P³² oligomer of interest and a perfectly complementary UC oligomer. Then determine the DK profile for the perfect match stringent P³² oligomer duplexes, and the t.5d value for the SF perfect match oligomer duplexes. Such a P³² oligomer perfect match SF duplex t.5d value is here termed a perfect match SF duplex t.5d value, or a PM t.5d value. (c) Prepare a stringent P³² oligomer of interest perfect match oligomer duplex prep and isolate the P³² oligomer of interest oligomer molecules which are associated with the stringent P³² oligomer FF duplexes. Stringently re-hybridize these isolated P³² oligomer FF molecules with the same UC oligomer used to make them, to produce a re-hybridized stringent P³² oligomer FF duplex prep. Determine the DK profile for these P³² oligomer FF duplexes and obtain one or more measures of the t.5d value or values associated with this P³² FF duplex profile. Such t.5d values are here termed FF duplex profile t.5d values or FF t.5d values. (d) Compare the DK profiles and the t.5d values for the PM, MM, and FF stringent P³² oligomer duplexes. Then determine whether the FF t.5d value or values is greater than, less than, or equal to, the MM t.5d value. Further, determine the fraction of the FF DK profile, which is greater than, less than, or equal to the MM t.5d value.

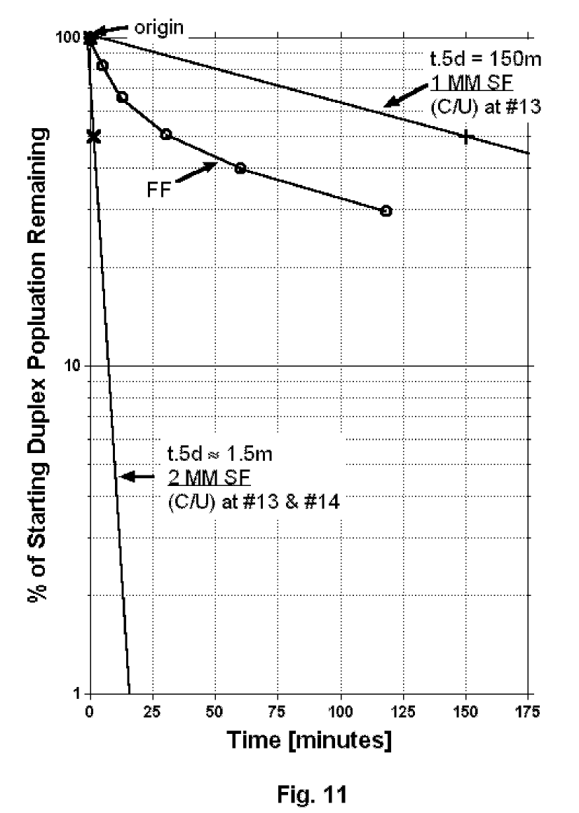

FIGS. 10 and 11 present the DK profiles used to estimate the extent of damage associated with the stringent FF P³² oligomers for a P³² dT₃₅ prep and a P³² N=24 biological sequence oligomer prep. The t.5d values presented in these figures represent the DK profile t.5d values for stringent SF duplexes at the specified analysis temperature and solution conditions.

The stringent FF P³² duplex DK profiles were measured under the conditions specified in each figure. For practical reasons, the stringent P³² oligomer SF duplex DK profiles for the mismatch and perfect match P³² oligomer duplexes were measured under different DK analysis temperatures in HDB. The t.5d values for these other temperature conditions were then adjusted to reflect the temperature condition specified in the figure. This process can be illustrated for the stringent SF P³² dT₃₅·dA₃₅ oligomer duplexes as follows. (i) Measure for the P³² dT₃₅·dA₃₅ oligomer duplex prep of interest, the stringent SF P³² dT₃₅·dA₃₅ oligomer duplex DK profile at 48.8° C. in HDB, and then determine the 48.8° C. t.5d value. The 48.8° C. t.5d value for the perfect match P³² dT₃₅·dA₃₅ SF oligomer duplexes in HDB was about 63 m. (ii) Convert the 48.8° C. measured t.5d value to the 45° C. t.5d value by using the relationship (45° C. t.5d value)=(48.8° C. t.5d value) (3.3)^{x}, where the 3.3 represents the measured P³² dT₃₅·dA₃₅ SF duplex associated (t.5d fold change/° C.) value discussed earlier, and X is equal to (the reference temperature−the adjusted to temperature). Here, the reference temperature is 48.8° C., the adjusted to temperature is 45° C., and X=3.8° C. Here then, (the 45° C. t.5d value)=(63 m) (3.3)^{3.9}=5884 m. Note that in adjusting from a 45° C. reference temperature to a 48.8° C. adjusted to temperature, X=3.8° C. The adjusted t.5d value is obtained using the (fold change in t.5d/° C.) value of (3.33 fold change in t.5d/° C.), measured for P³² dT₃₅·dA₃₅ SF duplexes in HDB (see Table 6 (8)). The (fold change in t.5d/° C.) value for a homogeneous oligomer duplex population is equal to the (fold change in k_{d}/° C.) value which is associated with the classic plot of (log k_{d} versus 1/T) for the oligomer duplex population. Such a (log k_{d} versus 1/T) plot is expected to be linear over a broad temperature range, and has been reported to be linear over a 20° C. range for oligomer duplexes. For FIGS. 10 and 11 the temperature range over which a t.5d value was adjusted, was 4° C. or less.

FIG. 10 presents the extent of damage analysis for isolated P³² dT₃₅ oligomer molecules, which are associated with stringent FF P³² dT₃₅·dA₃₅ oligomer duplexes. For simplicity in this discussion, these P³² dT₃₅ oligomer molecules will be termed FF P³² dT₃₅ oligomers or FF P³² oligomers, and the stringent FF P³² dT₃₅·dA₃₅ oligomer duplexes will be termed FF P³² dT₃₅ duplexes or FF duplexes. The FF P³² dT₃₅ duplex DK profile is curved and clearly represents the DK profile of a heterogeneous duplex molecule population. The DK profile clearly indicates that certain sub-populations of FF P³² dT₃₅ duplexes dissociate much faster than others, and therefore have much smaller t.5d values than others. Overall, the DK profile indicates that the FF P³² dT₃₅ duplex molecule population can be divided into at least three main sub-populations. Here, these sub-populations will be termed the fast, intermediate, and slow sub-populations. Note that each sub-population is very likely composed of a heterogeneous population of FF duplex molecules. The fast FF duplex fraction is comprised of roughly 25% of the total FF duplex molecule population, and a rough estimate indicates that the t.5d value for this sub-population is at most 3-5 m. The FF P³² dT₃₅ oligomer molecules associated with this fast fraction sub-population are damaged more extensively than the intermediate or slow sub-population FF P³² dT₃₅ oligomer molecules. The extent of damage associated with these fast FF dT₃₅ oligomer molecules is roughly similar to that expected for stringent P³² dT₃₅·(dA₃₃X₂) two mismatch duplex molecules. The intermediate fraction sub-population represents roughly 30 to 40% of the total FF duplex molecule population, and has an estimated t.5d value of very roughly 15 m to 20 m. The extent of damage associated the FF P³² dT₃₅ oligomer molecules in this intermediate sub-population, is roughly intermediate between that observed for a one and two mismatch situation. The slowest sub-population of FF duplex molecules represents roughly 30 to 40% of the total FF duplex molecule population. The t.5d value for this sub-population of FF duplexes is roughly equivalent to the one mismatch t.5d value. This indicates that the extent of damage for the sub-population of FF P³² dT₃₅ oligomer molecules, which are associated with this slow fraction, is roughly equivalent to that for a one mismatch duplex.

Essentially all of the FF P³² dT₃₅ oligomer molecules are damaged to a significant extent. A rough estimate of the average extent of damage per N=35 FF P³² dT₃₅ oligomer molecule, is equivalent to about 1.5 (C/T) mismatches per FF molecule. Note that the (C/T), (U/C) (A/A), (T/T), and (C/A) mismatches significantly destabilize an oligomer duplex while (G/T) and (G/A) mismatches are much less destabilizing. Note in addition that the effect of a (C/T) mismatch in the number 9 position of a dT₃₅·dA₃₄·C₁ duplex is almost as effective in destabilizing the duplex, as a (C/T) mismatch at position 18 which is directly in the middle of the N=35 dT₃₅ duplex. The measured t.5d value for a P³² dT₃₅ duplex with a position 9 (C/T) mismatch is only 1.1 times slower than for a position 18 (C/T) mismatch duplex (see table 8).

The stringent FF P³² dT₃₅ oligomer molecule sub-population comprises about 0.2 of the total P³² dT₃₅ oligomer prep. Thus, one out of five P³² dT₃₅ oligomer molecules is an FF P³² dT₃₅ oligomer molecule. If the average FF P³² dT₃₅ oligomer molecule is associated with 1.5 bad bases per molecule, then there are 30 bad bases present per 100 dT₃₅ oligomer prep molecules or 30 bad bases per 3500 total bases in the 100 dT₃₅ molecules. This is an error rate of about 0.9 base per 100 T nucleotides. Here, a bad base can be a chemically modified or damaged nucleotide or, normal nucleotide, which is not thymidine nucleotide.

Similar results were obtained for a P³² biological sequence oligomer prep. FIG. 11 presents the extent of damage analysis for isolated N=24 Biological Sequence (BS) P³² oligomer molecules which are associated with stringent FF N=24 BS P³² oligomer duplexes. For simplicity, these N=24 BS P³² oligomer molecules will be termed FF P³² BS oligomer molecules or FF P³² oligomers, and the stringent FF P³² BS oligomer duplexes will be termed FF P³² BS oligomer duplexes or FF P³² oligomer duplexes or FF duplexes. The FF P³² oligomer duplex DK profile of Table 12 is curved and clearly represents the DK profile of a heterogeneous oligomer duplex population. The DK profile clearly indicates that certain sub-populations of FF P³² oligomer duplexes dissociate much faster than others, and therefore have much smaller t.5d values than others. Overall the profile indicates the total FF P³² dT₃₅ oligomer duplex molecule population can be roughly divided into two main sub-populations, one which dissociates fast, and one which disassociates slow. Each sub-population appears to represent 30% to 50% of the total. Note that each of these FF P³² oligomer duplex sub-populations is very likely heterogeneous. The estimated t.5d value for the fast sub-population is roughly 5 to 10 m, a value, which is near that for the two mismatch duplex t.5d value. The estimated t.5d value for the slow sub-population is roughly equivalent to that of the one mismatch duplexes. Clearly, the fast sub-population associated FF P³² oligomer molecules are significantly more damaged than the slow sub-population associated FF P³² oligomer molecules.

Essentially, all of the N=24 BS FF P³² oligomer molecules are damaged to a significant extent. A rough estimate of the average extent of damage per FF N=24 BS P³² oligomer molecule is that the average damage is equivalent to about 1.5 (C/U) mismatches per oligomer molecule. Here, the U represents deoxyU. As discussed, the (C/U) duplex strongly destabilizes the duplex. Note further that the (C/U) mismatch is in the middle of the duplex at position 13, where the mismatch will have the maximum duplex destabilization effect. The stringent FF N=24 BS P³² oligomer molecule sub-population comprises about 0.18 of the N=24 BS P³² oligomer prep. If there are 1.5 damaged or bad bases per average FF P³² oligomer molecule, then there are 27 bad bases per 100 total BS P³² oligomer molecules, or 27 bad bases per 2400 total bases in the 100 N=24 oligomer molecules. Here, a bad base can be a chemically modified or damaged nucleotide, or a normal nucleotide, which is inserted in the wrong position.

Note that the above-described methods can be used to further fractionate and characterize the FF in order to obtain more precise extent of damage estimates.

Table 3 summarizes the stringent P³² oligomer duplex analyses of 63 different chemically synthesized and purified N oligomer preparations. These oligomer preparations were obtained from about 12 different sources, including a variety of commercial oligomer sources. The methods used for the synthesis and purification of these N oligomers were state of the art methods then and now. Table 3 lists for each of these purified N oligomer preps the fraction of stringent P³² oligomer duplex molecules, which are in the FF. Such measured FF values varied from nearly 95% to about 5%. For each of the 63 different DK analysis profiles, the FF t.5d was clearly very much faster than the t.5d of the corresponding SF. For many of these FF duplexes it can be roughly estimated that the FF duplex dissociate roughly 100 times faster than their corresponding stringent SF duplexes. This was the case for all analyses, which could be estimated. A 100 fold faster t.5d value is consistent with an extent of damage in the FF associated P³² oligomer molecules which is equivalent to one mismatched base or so. Overall then, all of the estimates of extent of damage in the purified N oligomer prep are consistent with an extent of damage of roughly one mismatch or more per FF oligomer molecule.

As discussed earlier, prior art generally believes and practices that prior art synthesized and purified N oligomer molecule populations which contain only oligomer molecules which have the intended N value, are homogeneous populations of oligomer molecules. Prior art further believes and practices that prior art synthesized oligomer preps can be highly purified for oligomer molecules which have the intended N value, and that the highly purified N oligomer prep can be shown to be composed of 95% or more oligomer molecules with the intended N value, and 5% or less oligomer which has an N−1 value, and no detectable N−X oligomer, where X=2 or more. Thus, prior art also believes and practices that the most highly purified N oligomer preps can be slightly heterogeneous, and that the heterogeneity is largely caused by the presence of about 5% or less N−1 oligomer. It has been demonstrated by the prior art that the population of N−1 oligomers in a purified biological sequence N oligomer prep is itself a heterogeneous population of N−1 molecules. Such an N−1 oligomer population has been shown to be composed of many different sub-populations of N−1 molecules, and each sub-population contains an internal deleted base at a particular position in the oligomer sequence. When such an N−1 molecule forms a stringent duplex, the resulting duplex contains an internal unpaired base and the duplex has the dissociation characteristics of a mismatched duplex, and will dissociate very rapidly, relative to a perfectly base pair matched duplex. Such an internal deletion duplex will also dissociate much more rapidly than a perfect matched duplex composed of an N oligomer and an N−1 oligomer which does not contain a internal deletion. No internal mismatch deletion effect will be observed for a homopolymer duplex composed of an N−1 oligomer and an N oligomer.

The above indicates that for a highly purified biological sequence N oligomer prep which contains N−1 oligomers, the N−1 oligomers will form stringent N−1 duplexes which contain an internal unpaired base. The effect of the N−1 duplex unpaired base on the duplex t.5d value is similar to that of a mismatched base on the duplex t.5d value. Stringent N−1 oligomer duplexes will be part of the FF in DK analysis fractionation.

For a highly purified homopolymer sequence N oligomer prep which contains dT₃₄ or dA₃₄ oligomers, the oligomers do not form stringent dT₃₄·dA₃₄ duplexes which have an internal unpaired base. The resulting duplexes have t.5d values, which are close to those of the stringent dT₃₅·dA₃₅ oligomer duplexes, which have perfect end to end base pair matching. For P³² labeled stringent dT₃₅·dA₃₅, dT₃₅·dA₃₄, and dT₃₄·dA₃₄ oligomer duplexes, the 50° C. HDB t.5d values are respectively about 15 m, about 11 m, and about 7.5 m. For a DK analysis of stringent duplexes for a P³² dT₃₅ oligomer prep, neither the P³² dT₃₅·dA₃₄, or P³² dT₃₄·dA₃₄ duplexes present will dissociate fast enough to be included in the homopolymer oligomer molecule FFs which are listed in Table 3. Note also that the vast majority of P³² biological and homopolymer oligomer preps analyzed, contained no detectable N−1 P³² oligomer. Note also that for almost all of the analyzed oligomer preps, the measured size of the stringent FF is much greater than the small amount of N−1 oligomers which the prior art believes is present in purified N oligomer preps.

Prior art generally believes and practices that prior art synthesized and purified N oligomer molecule populations, which contain only oligomer molecules which have the intended N value, are highly homogeneous populations of molecules. Anecdotal evidence indicates that the prior art believes such N molecule populations to be 97-99% homogeneous, and believes also that the major source of heterogeneity in highly purified N oligomer preparation is associated with the N−1 oligomer molecule population which is present in a N oligomer preparation. Prior art is aware that “bad” nucleotides can be present in N oligomer molecules, but generally believes that the rate of occurrence of such bad bases is low, and causes little damage or heterogeneity in the N oligomer molecule population. These prior art beliefs occur because the prior art methods used to characterize the purified N oligomer preparations have not detected significant heterogeneity in the N oligomer molecule population. The results of Table 3, and the above described determination of the extent of base damage associated with a significant fraction of the N oligomers in a purified N oligomer prep, show the invalidity of the prior art beliefs and practices concerning the homogeneity of the purified N oligomer molecules and their contribution to the heterogeneity of a purified N oligomer preparation. These results indicate the following. (a) All purified homopolymer and biological N oligomer preparations which contained no detectable N−1 oligomer molecules are significantly heterogeneous, and a significant fraction of the N oligomer molecules in the purified N oligomer preparations possess internally located bad or damaged bases. (b) All purified N oligomer preparations which contained a detectable N−1 oligomer fraction, are heterogeneous to a much greater extent than can be accounted for by the presence of the N−1 oligomer fraction. (c) All 63 purified N oligomer preparation analyzed were significant heterogeneous.

The reasons for the occurrence of the bad bases in the N oligomer molecules is not known. Such damage could be associated with many different oligomer synthesis factors including, but not limited to, the following. (i) The purity and homogeneity of the protected synthesis precursors. (ii) The stability of the synthesis precursors. (iii) Damage due to chemical side reactions during normal synthesis. (iv) Damage due to various aspects of the instrument efficiency and the synthesis process. (v) Damage due to the processing and purification of oligomers. (vi) Damage due to various aspects of the storage and use of the oligomers. (vii) Various other reasons. One or more of these (i)-(vii) items may contributed to the oligomer damage.

It is unlikely that such base damage can be prevented if it is not possible to detect the presence of the damaged bases in the oligomers. Prior art has not been able to detect such base damage and therefore could not determine the cause of the damage nor devise methods for preventing or minimizing the damage. The above described invention associated methods for the functional characterization make it possible to detect the presence of the base damage and can be used to develop approaches for eliminating or minimizing such damage, or simply making the base damage reproducible. The use of these improved functional characterization methods to detect and quantitate the presence of such base damage in oligomers or other nucleic acids, and the use of such methods to improve the quality of the synthesis oligomers or other nucleic acids, provide improved oligomer characterization results, relative to prior art oligomer characterization results, and are practices of the present invention.

The Determination of Whether for a Synthesized, Purified, N Oligomer Molecule Population the Measured Physical Chemical Properties are Equal to the Intended Physical Chemical Properties.

Chemically synthesized oligomers are designed to have an intended nucleotide length N, an intended nucleotide sequence, and an intended nucleotide composition. These intended oligomer properties then determine the intended physical chemical properties of the intended oligomer molecules. Further, the preparation of intended oligomer molecules is intended to be a homogeneous population of oligomer molecules which all have the same physical chemical properties. The intended physical chemical properties of the oligomer molecules determine the intended functional characteristics of the intended oligomer molecules. Such functional characteristics include, but are not limited to, the following. (i) The hybridization ability of the oligomers. (ii) The hybridization specificity of the oligomer molecules. (iii) The hybridization kinetics of the oligomer molecules. (iv) The stability of the hybridized oligomer duplex molecules. (v) The kinetics of dissociation of the hybridized duplex molecules.

A previous section describes the determination of improved results for the functional characterization of prior art synthesized and purified N oligomer molecule preparations which are, relative to the prior art produced functional characterization results, known to be significantly improved. However, while these functional characterization results can be known to be improved, they cannot be known to be the intended functional characterization results. In other words, the measured physical chemical properties associated with the synthesized and purified N oligomer preparation molecules, cannot be known to be identical to the intended physical chemical properties for the intended oligomer. This occurs because purified reference oligomer molecule preps, which are known to be essentially identical to the intended oligomer, are not used or available. In other words, purified reference “gold standard” oligomers are not used or available as references.

For the design of chemically synthesized oligomers the design context for the intended oligomer molecules nucleotide type (i.e., RNA or DNA), nucleotide sequence, nucleotide length, and nucleotide composition which is almost always used, is a naturally occurring biological nucleic acid molecule. The intended oligomer molecule is then equivalent to a biologically produced RNA or DNA oligomer molecule which has the intended nucleotide length, nucleotide sequence, and nucleotide composition. The physical chemical properties of such an intended biologically produced oligomer are the intended physical chemical properties of the chemical synthesis produced and purified N oligomer molecules. Further, the functional characteristics of such an intended biologically produced oligomer are the intended functional characteristics of the chemical synthesis produced and purified N oligomer molecules.

Therefore, it can be determined whether the measured physical chemical properties of the chemically synthesized, purified N oligomer molecules are the same as the intended oligomer molecule physical chemical properties by the following process. (a) Prepare essentially homogeneous preparations of biologically produced RNA or DNA oligomer molecules which have the intended nucleotide length, nucleotide sequence, and nucleotide composition. (b) Prepare essentially homogeneous preparations of biologically produced RNA or DNA molecules, which have the intended complementary nucleotide length, nucleotide sequence, nucleotide composition, and degree of nucleotide sequence complementarity. (c) Use the above-described methods to determine the physical chemical properties and functional homogeneity and functional characteristics for the intended biologically produced oligomer molecules. (d) Use the same methods to determine the physical chemical properties and functional homogeneity and functional characteristics of the chemically synthesized, purified N oligomer molecule population. (e) Compare the measured improved results for the physical-chemical properties and functional homogeneity and functional characteristics of the biologically produced and chemically produced N oligomer molecule preps. When the measured functional characteristic values for the chemically synthesized stringent N oligomer prep SF are equal to the functional characteristic value for the biological N oligomer prep SF, the intended and actual functional properties and characteristics can be considered to be equal.

A variety of prior art methods are available for producing biologically produced RNA or DNA duplex or single strand molecules which have a specified nucleotide length, nucleotide sequence, and nucleotide composition (4). Herein biologically produced DNA or RNA refers to RNA or DNA preparations, which are produced in vivo or in vitro by biological nucleic acid polymerases. The use of biological polymerases to produce such RNA or DNA molecules ensures that the produced oligomer molecules contain very few misincorporated wrong bases or damaged bases. The incidence of damaged or bad bases in biologically produced DNA RNA molecules is known to be very much lower than the incidence in chemically synthesized and highly purified N oligomer molecules. Because of this the intended biologically produced RNA or DNA oligomer preps are much more homogeneous than are synthesized oligomer preps and can be considered to be essentially homogeneous.

One of skill in the art will be aware of a variety of prior art methods for producing the intended biologically produced, homogeneous, RNA or DNA oligomer preps, and will also be aware that logistically, such long oligomer molecule preps are easier to produce than such short oligomer preps.

Note that the above described determination of whether the improved measured functional homogeneity and functional characteristics for a chemically synthesized SF N oligomer are the same as those of the reference oligomer SF N oligomer, represents the practice of the present invention to obtain further improved results concerning the functional homogeneity and functional characteristics of the chemically synthesized SF N oligomer molecules.

Measurement of the Effect of Mismatched, Unpaired, or Damaged Nucleotides on the Oligomer Functional Characteristics.

Nucleic acid molecules often exist as double stranded or duplex molecules. The duplex molecule can be converted to two single strand molecules by a variety of means including, but not limited to, treatment with temperature, salt, pH, solvent type, and others. For a duplex molecule the component single strand molecules are held together by a combination of different non-covalent attractive forces. This non-covalent glue consists primarily of hydrogen bonding and different types of non-covalent attractive forces. The strength of the non-covalent attractive forces or glue, which holds the individual double strands together, is influenced by a variety of well known factors. These factors include, but are not limited to, the following. (i) The nucleotide sequence of each single strand molecule. (ii) The type of nucleotides present in each single strand molecule, i.e., whether they are RNA or DNA or modified or other nucleotides or mixtures of such. (iii) The integrity of the nucleotides present in each single strand molecule, i.e., whether the nucleotides are damaged or undamaged. (iv) The degree and perfection of base pair matching which is present in the duplex region, i.e., whether each base in one strand is paired with a base in the other strand, and whether a base in one strand is paired with a base in the other strand which is perfectly complementary, partially complementary, or not complementary at all. (v) The type and concentration of ions which are present in the duplex containing solution. (vi) The solvent type and concentration in the duplex containing solution. (vii) The pH of the duplex containing solution. (viii) The presence of other organic or non-organic additives in the duplex containing solution.

Prior art commonly characterizes a particular nucleic acid duplex prep by determining a measure of the thermal stability of the duplex molecule population under controlled solution conditions. A variety of such prior art methods are available. The most commonly used prior art duplex thermal stability determination method is the earlier discussed Optical Method, or OM. Here, the OM measure of the duplex thermal stability is the temperature at which one half the DNA being analyzed is in the single strand state, and one half is in the double strand state. This OM thermal stability measure is termed the Tm for the duplex under the OM measurement conditions. As discussed earlier, the duplex Tm value is concentration dependent, and the duplex Tm value represents the temperature at which the number of duplex molecules dissociating into single strands in time period X, is equal to the number of duplexes formed by hybridization during the same time period. As discussed, the oligomer duplex prep equilibrium constant can be determined at the Tm if the total oligomer single strand concentration is known for the OM analysis. It is well known by the prior art that a duplex prep OM Tm value reflects the temperature at which the above described equilibrium occurs, and that the Tm value is an indirect measure of the duplex temperature stability.

Prior art often uses such duplex Tm values for predicting and optimizing certain functional characteristics of oligomer applications of many kinds. These applications include, but are not limited to the following. (a) Nucleic acid hybridization based assays, which are utilized to directly detect infectious agent RNA or DNA of all kinds, or cellular RNA or DNA of all kinds, or other RNA or DNA, or other nucleic acids of all kinds. Here, prior art often adjusts the measured Tm value for the duplex of interest to the Tm expected for the duplex under the application conditions. These applications almost always require the formation of a stable nucleic acid duplex at a temperature which is significantly below the application condition Tm value for the hybridized duplex of interest. Examples of such applications are DNA probe tests for infectious organisms, gene expression tests, and mismatch detection or SNP tests. (b) Nucleic acid hybridization based oligomer applications, which are utilized to indirectly detect infectious agent RNA or DNA of all kinds, or cellular RNA or DNA of all kinds, or other RNA or DNA or other nucleic acids of all kinds. These applications often involve the formation of a nucleic acid duplex at temperatures, which are equal to or near the Tm of the duplex of interest. Here, prior art often adjusts the measured Tm value for the duplex of interest to the Tm expected for the duplex under application conditions. However, such a prior art adjustment is generally problematic because prior art has no valid reference for what the Tm of the duplex would be under the application conditions. The application conditions often contain components, which make it impossible to measure an OM duplex Tm under the application conditions.

An example of this is the widely used PCR method. For a PCR assay the primer oligomer molecule is complementary to, and hybridizes with the primer site in the target DNA molecule to be amplified, in order to form a primer·target duplex which can be utilized by the DNA polymerase to begin DNA synthesis of the amplicon. Such synthesis starts from the 3′ end of the duplex primer molecule. Each primer is generally present in the PCR mix at around 10⁻⁶M concentration, and the primer is in great molar excess to the target at all times during the PCR process. The PCR amplification mix contains a DNA polymerase, a target DNA template, monovalent cation salt, divalent cation salt, precursor nucleotide triphosphate salts, a buffer, primers and other additives. Many of these components can affect the Tm of the primer-target duplex, and also make it impossible to determine an optical Tm value for the primer·target duplex under the application conditions.

For both the above described prior art applications for which prior art uses OM measured Tm values determined under standard conditions to predict and optimize for certain oligomer functional characteristics, the oligomer·target duplex t.5d under the application conditions is a key functional characteristics which has a great influence on the effectiveness of the oligomer application use. For both the described applications, the duplex t.5d value or values for both the oligomer duplex or duplexes intended to be detected, and the oligomer duplexes not intended to be detected, are important for the successful and/or optimum application performance. Here, the ability to differentially and specifically detect the analyte of interest is determined by the absolute and relative t.5d values associated with the intended oligomer·target duplexes and the unintended and unwanted oligomer·target duplexes under the application conditions. Such absolute and relative t.5d values are rarely, if ever, determined and directly taken into consideration for prior art oligomer application's such as DNA probe assays, gene expression assays, SNP detection assays, PCR assays in general, and many other applications. The determinations of such absolute and/or relative oligomer·target duplex t.5d values would provide, relative to the prior art, an improved basis for evaluating and interpreting and improving, existing prior art oligomer applications, and an improved basis for designing and optimizing new oligomer applications.

Note that the oligomer FH value, FD value, and association constant (kₐ) value, are also important functional characteristics of an oligomer application. The overall performance of an oligomer application is dependent on the interaction between these various functional characteristics. Clearly the determination and consideration of the absolute and relative FH, FD, kₐ, and t.5d values associated with both the intended and unintended or unwanted oligomer·target duplexes of an oligomer application, provides an even greater improvement in the ability to evaluate, interpret, and improve existing prior art oligomer applications, and an even greater improved basis for designing, evaluating, and optimizing, new oligomer applications.

Purified N oligomer molecules are intended to have functional characteristics which are associated with oligomer molecules of the intended nucleotide sequence, nucleotide length, and nucleotide composition. Further, purified N oligomer molecule populations are intended to be composed of a homogeneous or essentially homogeneous population of oligomer molecules which all have the same physical chemical characteristics and functional characteristics. As discussed earlier, all 63 chemically synthesized and purified N oligomer molecule preps evaluated thus far contain a significant fraction of N oligomer molecules, which possess damaged nucleotides. As shown in Table 3, different purified N oligomer preps contained from about 5% to over 98% damaged oligomer and the “average” N oligomer prep contains about 25% bad oligomer. Here, such damage to the oligomer indicates that the damaged N oligomer does not have the intended nucleotide sequence and/or nucleotide composition, and/or nucleotide integrity. Such a damaged N oligomer does not hybridize with the intended target to form the intended oligomer·target duplex molecule. A duplex molecule, which contains a damaged N oligomer, may contain one or more of the following damaged duplex regions. (i) A mismatched base pair. (ii) An unpaired base. (iii) A base pair involving a damaged or modified nucleotide or base. As discussed in the earlier section on the estimation of the extent of damage present in the damaged oligomers, stringent oligomer duplex molecules which are associated with damaged oligomers, dissociate much faster than do stringent P³² oligomer duplexes which are not associated with damaged oligomers. Every prior art purified N oligomer prep evaluated thus far, has contained a significant fraction of damaged N oligomers. Further, the stringent P³² N oligomer duplex molecule prep for each of the evaluated prior art N oligomer preps, was composed of a heterogeneous population of stringent P³² N oligomer duplexes. For all of these prior art purified N oligomer preps the heterogeneous stringent P³² N oligomer duplex molecule population contained a significant N oligomer duplex fraction which dissociated very rapidly (i.e., the FF), relative to a second N oligomer duplex fraction (i.e., the SF). It appears that for a typical stringent P³² N oligomer prep, the FF dissociates about 100 times faster than the SF.

As discussed earlier, it appears that virtually all prior art chemically synthesized and purified N oligomer populations contain a significant population of unintended and unwanted N oligomer molecules, and a significant population of what appears to be intended N oligomer molecules. The N oligomer molecule population in each of these prior art produced and purified N oligomer preps, is therefore heterogeneous, and all of the N oligomer molecules in such a prep do not have the same physical chemical properties or functional characteristics. As has been discussed, the oligomer functional characteristic FH and kₐ values are relatively insensitive to significant extents of oligomer damage. For example the presence of 10% or so mismatched base pairs in an oligomer duplex molecule, results in no change in the FH value, and a roughly twofold reduction in the kₐ value for the oligomer, relative to an undamaged oligomer. For this mismatched oligomer the FD value can be greatly affected or not, depending on the conditions chosen for the DK analysis. In contrast, the oligomer associated t.5d value is virtually always very significantly decreased by the presence of oligomer damage. Thus, significant N oligomer damage will have little effect on the functional characteristics FH and kₐ for an N oligomer application, but will have a very large effect on the functional characteristic t.5d value for the N oligomer application.

The presence of significant amounts of damaged N oligomer molecules in an N oligomer application generally has little effect on the FH and kₐ functional characteristic values for the oligomer application. Thus, the presence of differing amounts of such damaged N oligomer molecules in different oligomer lots will generally not cause significant differences in the N oligomer application functional characteristic FH or kₐ values due to oligomer lot differences. In contrast, the presence of significant amounts of damaged N oligomer molecules in an N oligomer application can greatly affect the t.5d functional characteristic value for a significant fraction of the N oligomer duplexes in the oligomer application, and cause a significant degradation in the oligomer application performance characteristics. Here, the greater the fraction of damaged oligomer molecules present, the greater the degradation of the oligomer application performance characteristics. Further, the presence of significantly different amounts of damaged N oligomer in different lots or preps of purified N oligomers, can result in significant differences in the oligomer application performance at different times. A discussion of the effect of a known degree of N oligomer damage on an N oligomer duplex SF t.5d value is presented below.

It is useful to discuss the results of a standard prior art OM method Tm determination for N=24, biological sequence, purified N oligomer duplex molecules which have, no intended mismatches (a), one intended (C/U) mismatch at position #13 (b), and two intended (C/U) mismatches at positions #13 and #14 (c). Note that in 1M NaCl and at a total single strand oligomer concentration of 4×10⁻⁶M, the measured Tms are 76.7° C. (a), 70.7° C. (b), and 64.7° C. (c). Significant destabilization of a duplex is caused by (C/U), (A/A), (T/T), (C/T), and (C/A) mismatches, while (G/T) and (G/A) mismatches only slightly destabilize a duplex. Here, the mismatch is a (dC/dU) mismatch (27). One (C/U) mismatch then, causes a 6° C. decrease in the OM measured Tm.

Such prior art OM analyses tacitly believe and practice that each analyzed N oligomer duplex prep is composed of an essentially homogeneous population of N oligomer duplex molecules which have the intended nucleotide sequence, the intended nucleotide composition, the intended nucleotide integrity, and therefore the intended same physical chemical properties and functional characteristics. The use of such prior art measured OM Tm values for predicting the functional characteristics of the N oligomer molecules for different oligomer application conditions, depends on the validity of this tacit prior art belief and practice. Note that the optical melting profiles of the Perfect Match (PM) duplexes (a), and the Single Mismatch (MM) duplexes (b) overlap significantly, as do the single MM (b) and double (C) duplex melting profiles. The PM (a) and double MM (c) melting profiles also overlap to a lesser extent.

As discussed, prior art purified N oligomer preps are not composed of an essentially homogeneous population of N oligomer molecules. All such prior art N oligomer preps checked thus far except one have been composed of a significant fraction of damaged N oligomer molecules and a significant fraction of apparently undamaged N oligomer molecules. The exception N oligomer prep appeared totally heterogeneous. It is likely that all or virtually all prior art purified N oligomer preps are similarly heterogeneous and as a consequence, stringent P³² N oligomer duplex preparations prepared using these prior art heterogeneous N oligomer preps, are also heterogeneous, and composed of stringent P³² N oligomer duplex molecules which are associated with damaged N oligomers and stringent P³² N oligomer duplex molecules which are not associated with damaged oligomers. Further, the damage associated stringent P³² N oligomer duplex molecule population appears to dissociate about 100 times faster than the undamaged stringent P³² N oligomer duplex molecule population.

Table 8 presents DK profile results for a variety of different stringent N oligomer duplex molecule preparations. For each separate stringent P³² N oligomer duplex preparation the stringent % FF value, the stringent % SF value, and stringent SF t.5d value were determined as described earlier, and the results presented in Table 8. Many of these stringent P³² N oligomer duplexes are associated with intended and known quantitative levels of nucleotide mismatches at known positions in the oligomer duplex molecules, or known quantitative levels of unpaired nucleotides which are external to the duplex region or internal to the duplex region. These mismatched (MM) and unpaired nucleotides represent damage, which can actually be present in an N oligomer prep.

TABLE 8
Determination of Stringent P³² N Oligomer Duplex Prep DK Profile and %
FF and % SF and T.5d Values for Oligomer Duplex Molecules Associated with Unpaired or Mispaired Bases

| | | Complementary | Mismatched or Damaged | Values Determined For | | |
|---|---|---|---|---|---|---|
| P³² Oligomer | | Oligomer | Base and Type | % FF | % SF | SF t.5d (m) |
| (1) | dT₃₅ | dA₃₅ | None | 18 | 82 | ^{(a)}5230 (45° C.) |
| | dT₃₅ | dA₃₄ | One Unpaired T at End of dT₃₅ | 18 | 82 | 3456 (45° C.) |
| | | 9 | | | | |
| | dT₃₅ | dA₃₄.C₁ | One Mispaired (C/T) at Position #9 | 19 | 81 | 40 (45° C.) |
| (2) | (a) dT₃₅ | dA₃₅ | None | 30 | 70 | ^{(a)}4813 (45° C.) |
| | 5 | | | | | |
| | (b) dT₃₄.A₁ | dA₃₅ | One Mispaired (A/A) at Position #5 | 20 | 80 | 180 (45° C.) |
| | 5 | | | | | |
| | (c) dT₃₄.G₁ | dA₃₅ | One Mispaired (A/G) at Position #5 | 19 | 81 | 212 (45° C.) |
| (3) | (a) dT₃₅ | dA₃₅ | None | 12 | 88 | 4814 (45° C.) |
| | | 9 | | | | |
| | dT₃₅ | dA₃₄.C₁ | One Mispaired (C/T) at Position #9 | 12 | 88 | 48 (45° C.) |
| | | 18 | | | | |
| | dT₃₅ | dA₃₄.C₁ | One Mispaired (C/T) at Position #18 | 12 | 88 | 48 (45° C.) |
| (4) | dA₃₅ | dT₃₅ | None | 32 | 68 | 4617 (45° C.) |
| | | 9 | | | | |
| | dA₃₅ | dT₃₄.G₁ | One Mispaired (G/A) at Position #9 | 28 | 72 | 72 (45° C.) |
| | | 18 | | | | |
| | dA₃₅ | dT₃₄.G₁ | One Mispaired (G/A) at Position #18 | 30 | 70 | 75 (45° C.) |
| (5) | (a) dA₃₄ | dT₃₄ | None | 23 | 77 | ^{(a)}2857 (45° C.) |
| | dA₃₄ | dT₃₅ | One Unpaired T at End of dT₃₅ | 24 | 76 | 4227 (45° C.) |
| | (b) dT₃₄ | dA₃₄ | None | 19 | 81 | 2935 (45° C.) |
| | dT₃₄ | dA₃₅ | One Unpaired A at End of dA₃₅ | 23 | 77 | 4227 (45° C.) |
| (6) | dT₁₇.ABS.dT₁₇ | dA₃₅ | One Internal Unpaired A at Position | 20 | 80 | ^{(a)}23 (45° C.) |
| | | | #18 (ABS = Abasic Site) | | | |
| (7) | (a) N = 24 | N = 24 BS PM | None | 15 | 85 | ^{(a)}78,459 (50° C.) |
| | BS | | | | | |
| | (b) N = 24 | N = 24 BS PM | A linker is placed between | 20 | 80 | 7019 (50° C.) |
| | BS | | phosphates 13 and 14 in the modified | | | |
| | Modified | | N = 24 oligomer, and creates a bulge | | | |
| | (c) N = 24 | N = 24 BS PM | One (C/U) Mismatch is Present at | 22 | 78 | 794 (50° C.) |
| | BS 1 | | Position #13 | | | |
| | Mismatch | | | | | |
| | (d) N = 24 | N = 24 BS PM | Two Adjacent (C/U) Mismatches | 20 | 80 | 144 (50° C.) |
| | BS 2 | | Present at Positions 13 and 14 | | | |
| | Mismatches | | | | | |
| (8) | (a) N = 21 | N = 51 Composed | 30 unpaired As at 3' end BS region is | 18 | 82 | ^{(b)}5348 (46° C.) |
| | DNA | of BS N = 21 and | designed to be perfectly matched | | | (0.12 M Na⁺) |
| | BS with | dA₃₀.NH₂ at the | | | | |
| | 3′ | 3′ End. BS is PM | | | | |
| | Biotin | | | | | |
| | DNA | | | | | |
| | (b) As (a) | As (a) except that | One base deletion in the | 16 | 84 | 38.4 (46° C.) |
| | | BS = 20 and | complementary strand which results | | | (0.12 M Na⁺) |
| | | contains a one | in a duplex bulge, and 30 unpaired | | | |
| | | base deletion at | As at 3′ end | | | |
| | | Position #9 | | | | |

All t.5d values measured at or adjusted to the DK analysis condition of:
^{(a)}HDB (0.135 M Na⁺) and 45° C. or 50° C.
^{(b)}0.12 M Na⁺ and 46° C.

The MM nucleotide represents a damaged oligomer situation where a normal nucleotide is incorporated into an oligomer at the wrong position. The unpaired nucleotides represent a situation where a deletion has occurred in an N oligomer molecule, or a depurinated nucleotide exists in an N oligomer molecule. A situation which directly represents a modified or damaged nucleotide is not included in the study. However, the literature suggests that the MM and unpaired nucleotide situations serve as a reasonable surrogate for the modified or damaged base situation, and that generally similar results will be obtained. Generally, the presence of modified nucleotides in the N oligomer duplex will result in a decrease in the duplex stability. However, the presence of certain modified nucleotides in the N oligomer duplex will increase the stability of the duplex. While such a modified oligomer duplex was not included in Table 8, the methods used here can readily be used to analyze the effect of damage on such N oligomer duplexes. The t.5d results presented in Table 8 for a particular oligomer duplex type were obtained using the HDB (0.135 M Na⁺) DK analysis solution, except for the duplex represented in Table 8 (8) where a 0.12 M Na⁺ DK solution was used. The t.5d values for a particular duplex type were normalized to one temperature for easy comparison. This normalization was done as described earlier. A general summary of the Table 8 results is presented in Table 9.

For each of the oligomer duplex types represented in Table 8, the values for the functional characteristics FH, FD, kₐ, and t.5d (or k_{d}), were determined for an oligomer of interest under the DK analysis conditions. In addition, a quantitative value for the amount of damage oligomer (i.e., the % FF), and apparently undamaged oligomer (i.e., the % SF), is presented for the oligomer of interest. The values for the oligomer functional characteristics FD, kₐ, and t.5d (or k_{d}), are very likely to be different for different oligomer application conditions than for the Table 8 DK analysis conditions.

TABLE 9
General Summary of Effect of Oligomer Damage on the Oligomer Duplex
t.5d Value

| | | | Extent of Decrease in t.5d | |
|---|---|---|---|---|
| | | | Value Caused By Damage | |
| Stringent Oligomer | | | (Undamaged t.5d | |
| Duplex | Known Duplex Damage | | Damaged t.5d) | |
| (1) dT₃₅.dA₃₅ | (a) | One unpaired base caused by the presence of an | (a) | ˜212 Fold |
| | | abasic (A/—) site at Position 18. | | |
| | (b) | One mismatched (C/T) base pair at Positions 9 | (b) | ˜100 Fold |
| | | or 18. | | |
| | (c) | One mismatched (G/A) base pair at Positions 9 | (c) | ˜63 Fold |
| | | or 18. | | |
| | (d) | One mismatched (A/A) base pair at Position 5. | (d) | ˜27 Fold |
| | (e) | One mismatched (G/A) base pair at Position 5. | (e) | ˜23 Fold |
| | (f) | Damaged duplex consists of otherwise | (f) | ˜1.7 Fold |
| | | undamaged truncated N − 1 dT₃₄ and dA₃₄. | | |
| | (g) | Damaged duplex consists of otherwise | (g) | ˜1.1 Fold |
| | | undamaged N dT₃₅ or dA₃₅ oligomer and | | |
| | | otherwise undamaged truncated N − 1 dA₃₄ or | | |
| | | dT₃₄ oligomer. The damage consists of one | | |
| | | unpaired nucleotide at the end of the duplex. | | |
| (2) N = 24 | (a) | Two adjacent (C/U) mismatches at Positions 13 | (a) | ˜545 Fold |
| Nucleotide pair DNA | | and 14. | | |
| duplex representing an | (b) | One (C/U) mismatch at Position 13. | (b) | ˜99 Fold |
| ˜50% GC biological | (c) | In one oligomer a linker is inserted in the | (c) | ˜11 Fold |
| DNA sequence | | oligomer phosphate backbone between | | |
| | | Positions 13 and 14 resulting in an N = 24 | | |
| | | oligomer with the linker inserted. This causes | | |
| | | the duplex to have a bulge at Positions 13 and | | |
| | | 14. | | |
| (3) BS DNA N = 21 | (a) | A one base deletion exists in the duplex region | (a) | ˜139 Fold |
| nucleotide pair duplex | | of the long oligomer at Position 9 resulting in | | |
| region and attached to one | | an unpaired (T/—) site. | | |
| strand of the duplex at the | | Note that the 3′ biotin and NH2 groups and | | |
| 3′ end is A30.NH2. A | | their associated linkers have only a small effect | | |
| biotin is attached to the | | on the duplex t.5d values. | | |
| N = 21 oligomer 3′ end | | | | |

However, the quantitative values for these oligomer functional characteristics can be readily determined for a wide variety of different oligomer application conditions. As an example, the quantitative values for the functional characteristics FH, FD, kₐ, t.5d (i.e., k_{d}) can be determined for a PCR, or other oligomer primer of interest, under the actual PCR application conditions, i.e., in a fully constituted PCR reaction solution, at the PCR cycle temperatures(s) of interest. Prior art oligomer functional characteristic determination methods cannot do this.

A variety of different types of oligomer damage can be present in a prior art purified N oligomer preparation. Such damage can be associated with the following. (i) Incorporation of the wrong nucleotide at a site. Such damage is represented by the mismatched nucleotides in Table 8. (ii) One or more deleted nucleotides in the oligomer. Such damage is represented by a deletion in Table 8. (iii) The presence of an abasic site in the oligomer. Such damage is represented by an abasic site in an oligomer in Table 8. (iv) A truncated N−X oligomer. Such damage is represented by N−1 oligomers in Table 8. (v) The presence of unnatural but intended chemical modification in the oligomer. Such damage is represented by the linker molecules, and biotin or NH₂ groups which are associated with Table 8 oligomers. (vi) The presence of unintended chemical modification in the oligomer. Such damage is not directly represented in Table 8. Table 9 provides a summary of the effect of these various damage types on an SF oligomer duplex t.5d value.

The overall pattern of the qualitative effect of different damage types generally agrees with prior art findings. These include but are not limited to, the following. (a) Different MM base pairs affect the duplex thermal stability to different extents. (b) The same MM at different positions can affect the duplex thermal stability to differing extents, and MMs close to either duplex end affect the duplex thermal stability the least. (c) Unpaired bases at the duplex end tend to stabilize the duplex. (d) Short duplexes are generally less stable than long duplexes. (e) The presence of abasic sites in the duplex have more effect on the duplex thermal stability than MMs. (f) The presence of a deleted nucleotide in the duplex region may be more destabilizing than MMs.

The SF t.5d values reported in Tables 3 and 8 differ from and are improved over, prior art reported oligomer duplex prep t.5d values. These differences and improvements include, but are not limited to, the following.

(i) The SF t.5d values differ from prior art oligomer duplex t.5d values in that the fraction of the analyzed oligomer which is represented by the SF t.5d value is known, and this fraction is almost always, if not always, equal to significantly less than one. In other words, the determination of these SF t.5d values does not assume that the analyzed oligomer molecule prep is homogeneous, but determines the degree of heterogeneity of the analyzed oligomer prep and takes this into consideration in the determination of the t.5d value for the oligomer prep. Because of this, the measured SF t.5d value for an oligomer prep can be known to represent an essentially homogeneous population of oligomer duplex molecules which are highly likely to have the intended oligomer physical chemical properties. In contrast, prior art DK analysis practice tacitly assumes that the prior art N oligomer molecule preparations are homogeneous or essentially homogeneous, and that the reported t.5d value represents all or essentially all of the oligomer molecules in the oligomer prep. In other words, prior art assumes the reported t.5d value associated with the oligomer prep represents an oligomer prep which is homogeneous, and does not determine the heterogeneity of the oligomer prep, and does not take into consideration the heterogeneity of the oligomer prep when interpreting the t.5d value for the prep. Because, as described in Tables 3 and 8, it is known that prior art N oligomer preps are often, if not always, significantly heterogeneous, it cannot be known whether a prior art reported oligomer t.5d value accurately represents the analyzed oligomer prep. Clearly then, the Table 3 and Table 8 presented N oligomer t.5d values as well as any other oligomer or other t.5d value which is obtained by taking into consideration the FH and/or FD, and the % SF and % FF values, is significantly improved relative to prior art reported oligomer or other t.5d values. Therefore, these Table 8 and Table 3 and other t.5d value determinations represent a practice of the invention, and represent practicing the invention to obtain improved oligomer and other functional homogeneity and functional characteristic measurements.

(ii) The effect of the presence of MM nucleotides and/or unpaired nucleotides in the stringent N oligomer duplex region on the SF t.5d value, is presented in Table 8 for oligomer duplex regions which range from 21 to 35 nucleotide pairs in length. Such an effect is expressed in terms of the fold change decrease or increase in the oligomer duplex SF t.5d value caused by the presence of MM or unpaired nucleotides in the stringent oligomer duplex regions. The quantitative value for the fold change caused by the presence of the MMs or the unpaired nucleotides, is equal to the ratio of, (the SF t.5d value of the oligomer duplex containing no MM or unpaired nucleotides measured under DK analysis condition X)÷(the SF t.5d value of the MM or unpaired nucleotide containing duplex measured under DK analysis condition X). The Table 8 reported magnitude of the SF fold change effect of even the least destabilizing nucleotide mismatches, is significantly greater than has been reported for prior art MM duplex t.5d fold change values. Note that, as discussed above, the prior art MM duplex t.5d fold change values cannot be known to represent the SF t.5d values of the oligomer preps, and also cannot be known to represent the t.5d value for the entire oligomer duplex preps. In addition, all or virtually all of the prior art reported MM duplex related t.5d values are associated with oligomer duplex regions which range from 2 to 19 base pairs in length. The above described absolute and relative improved functional characteristic values for MM and PM oligomer basis can be utilized to produce mismatch detection (i.e., SNP or mutation detection) or base damage detection oligomer applications which are greatly improved relative to such prior art oligomer applications. The determination of the improved absolute and relative PM and MM oligomer functional characteristic values presented in Table 8, and the use of such improved values for producing improved MM, PM, and damage detection oligomer applications constitute a practice of the current invention.

Consequence of the Invalidity of Tacit Prior Art Oligomer Duplex OM Analyses Method Assumption that the OM Analyzed Duplex Population is Homogeneous.

As discussed earlier, prior art oligomer duplex OM analysis practice tacitly assumes that the prior art analyzed oligomer duplex molecule population is composed of a homogeneous or essentially homogeneous population of oligomer duplex molecules. The validity of this tacit prior art assumption is required in order for the prior art use of the OM measured Tm values to be correct, and is further required for the validity or correctness of the analyzed oligomer duplex equilibrium constant derived from the OM analysis results. This is discussed below in the context of the OM analysis of prior art chemically synthesized and purified complementary N oligomer molecule populations.

A prior art Tm value determination by OM analysis for a particular N oligomer duplex requires the separate chemical synthesis and purification of the N oligomer of interest and a complementary N oligomer. Here, the complementary N oligomer is termed the CC N oligomer or CC oligomer. The above discussions indicate that it is highly probable that both the prior art oligomer of interest prep and the CC N oligomer prep are composed of a significant fraction of damaged N oligomers as well as undamaged N oligomers. Further, the sub-population of damaged N oligomer molecules in each prep is itself heterogeneous and is composed of oligomer molecules which are damaged to different extents. Because of the existence of significant populations of heterogeneous damaged oligomer molecules in each purified N oligomer prep used to produce the analyzed N oligomer duplexes, a prior art OM analyzed N oligomer duplex molecule population is highly likely to be significantly heterogeneous. Therefore, the prior art belief and practice that the prior art OM measured N oligomer duplex Tm values represent Tm values for essentially homogeneous oligomer duplex molecule populations, is highly likely to be invalid. In addition, the equilibrium constants derived from the prior art OM analysis results for the analyzed oligomer duplexes are highly likely to be significantly incorrect, absent some known compensating factor. This is discussed below.

When the prior art OM measured N oligomer duplex Tm value does not represent the Tm of an essentially homogenous N oligomer duplex prep, but does represent the Tm of a significantly heterogeneous N oligomer duplex prep, the following situation exists.

(a) The measured Tm value represents the temperature at which one half of the N oligomer molecules present in the analysis solution are in a dissociated state, and half of the N oligomer molecules are in a duplex form.

(b) The Tm value does not represent the equilibrium temperature for a homogeneous population of N oligomer duplex molecules, but represents the temperature at which half the oligomer molecules present are in a duplex form. When the oligomer duplex population being analyzed is not homogeneous, the measured Tm value represents the temperature at which multiple different equilibria interact in just the right way so that half the oligomer molecules present are in a duplex form.

(c) Because the damaged and undamaged N oligomer duplex molecules have significantly different equilibrium constants, and the damaged oligomer duplex molecule population is associated with many different equilibrium constants, absent knowledge of these equilibrium constants and the fraction of total oligomer associated with each different equilibrium constant, the utility of the standard prior art OM analysis measured Tm value is limited, and the true equilibrium constant for the analyzed oligomer duplex is unknown, and the prior art OM analysis derived equilibrium constant is incorrect.

(d) Because of (c), the desired equilibrium constant for the undamaged or SF N oligomer duplex molecule population cannot be determined from the total N oligomer single strand molecule concentration of the OM analysis. This occurs because in the presence of a heterogeneous N oligomer duplex population, neither the temperature at which half the SF duplex molecules are single stranded, nor the actual molar concentration of single strand SF N oligomer molecules in the OM analysis solution, is known for a prior art OM analysis of an oligomer duplex population.

(e) The above-described situation is further complicated by the existence of damaged N oligomers in both N oligomer preps used to form the analyzed N oligomer duplexes. For the earlier described DK analysis of stringent P³² N oligomer duplexes, it was possible to ensure that each damaged P³² N oligomer molecule could hybridize only to a complementary SF or undamaged N oligomer. This could be done by hybridizing the P³² N oligomer molecules with a significant molar excess of unlabeled complementary N oligomer under the described annealing conditions. This ensures that a stringent SF P³² N oligomer duplex essentially always involves an SF or undamaged complementary N oligomer molecule. This greatly simplifies the interpretation of the DK analysis results. For technical reasons it is not possible to do this for a standard OM Tm determination analysis, where equimolar amounts of each oligomer are present in the OM analysis solution. During a DK analysis when a P³² oligomer duplex molecule dissociates it does so irreversibly, that is the dissociated single strand P³² oligomer molecule does not re-hybridize with another complementary oligomer and dissociate again during the same DK analysis. In contrast, during an OM analysis a particular N oligomer molecule will re-hybridize and dissociate many times per minute during the course of one OM analysis. This creates a situation where a particular oligomer molecule which is associated with a damaged duplex at the start of the OM analysis, may quickly dissociate and re-hybridize to form an undamaged duplex. Alternatively, a particular oligomer molecule may be initially associated with an undamaged duplex and dissociate and re-hybridize to form a damaged duplex. Such a situation greatly complicates the ability to precisely interpret a measured OM analysis Tm value for heterogeneous populations of oligomer duplexes. In this context, the results shown in Table 3 indicate that a prior art purified N oligomer prep comprised of 80% SF oligomer molecules or less, is common. If both the N oligomer preps used for an OM analysis contain 80% SF oligomers, then for an equimolar mixture of the oligomer preps, the maximum fraction of damaged oligomer duplex which can be present at the start of the OM analysis is 0.4 of the total oligomer duplexes formed, and the minimum fraction is 0.2.

The prior art OM analysis Tm determination for oligomer duplexes is intended to measure the temperature at which one half of a homogeneous population of oligomer duplexes is dissociated, and at which the number of oligomer duplex molecules dissociating per time period equals the number of oligomer duplex molecules being formed by hybridization during the same time period. It is well known that if such an equilibrium temperature can be determined for a homogeneous oligomer duplex population, then the quantitative value of the equilibrium constant for the duplex population at that temperature can be readily calculated from the known oligomer concentration for the analysis (15,16). Determining the homogeneous oligomer duplex populations equilibrium constant under different OM analysis conditions allows the quantitative value for the Van't Hoff Transition Enthalpy (DH) of intermolecular oligomer duplex formation, and other thermodynamic parameters, to be determined for the homogeneous oligomer duplex population. The values for these thermodynamic parameters can then be used to predict the functional characteristics of the oligomer molecules for different oligomer application conditions. Prior art routinely practices this process using prior art OM analysis determined oligomer duplex Tm values, which are believed by the prior art to represent the Tm values for an essentially homogeneous oligomer duplex molecule population. As indicated herein the prior art belief in the homogeneity of the prior art OM analyzed duplex preps, is invalid because of the presence of significant heterogeneity of the analyzed prior art oligomer duplex molecule populations. The inability of this prior art OM method to determine the Tm of only the undamaged or SF oligomer duplex fraction which is present in a prior art oligomer duplex preparation significantly limits the utility and interpretation of the prior art measured oligomer duplex molecule population OM measured Tm values.

(f) Prior art at times utilizes OM analysis determined equilibrium constant values to determine either the kₐ or k_{d} value associated with the OM analyzed oligomer duplexes at the measured Tm. For a particular Tm value, (the equilibrium constant value for an analyzed homogeneous oligomer duplex molecule population)=(the kₐ value for the homogenous oligomer molecule population)÷(the k_{d} value for the homogeneous oligomer molecule population). When the equilibrium constant and the kₐ values are measured or known, then the k_{d} value can be determined. When the equilibrium constant and k_{d} values are measured or known, the kₐ value can be determined. The prior art determination of correct and valid kₐ and/or k_{d} values in this way requires measuring and knowing a correct and valid value for the equilibrium constant at the measured Tm. As discussed above, it is very reasonable to believe that essentially all prior art produced oligomer duplex OM analysis Tm values and the equilibrium constant values derived for these Tm values, are associated with heterogeneous populations of analyzed oligomer molecules. Further, prior art does not determine or know whether a particular purified N oligomer prep is homogeneous or not, and also does not determine the degree of homogeneity or heterogeneity of an oligomer prep. Therefore, at best, it cannot be known whether a prior art measured equilibrium constant is correct or incorrect, and therefore it cannot be known whether a kₐ or k_{d} value determined from a prior art equilibrium constant is correct or not. It is likely however, that essentially all such equilibrium constants, kₐs, and k_{d}s are also incorrect, but absent further information it cannot be known how far these values deviate from the correct values.

Prior art at times directly measures the kₐ and k_{d} values at a particular temperature for an analyzed oligomer prep, and uses these values to determine the equilibrium constant for the analyzed oligomer prep at the measurement temperature. Such a prior art determined equilibrium constant value can be correct only if the measured kₐ and k_{d} values for the analyzed oligomer preps represent kₐ and k_{d} values for an oligomer duplex prep, which is essentially homogeneous. Prior art does not measure or take into consideration the actual homogeneity or heterogeneity of the oligomers analyzed to determine the kₐ and k_{d} values. Therefore, it cannot be known whether such values represent homogeneous or heterogeneous oligomer preps. Therefore, equilibrium constants derived from such prior art determined kₐ and k_{d} values cannot be known to be correct or not. Although it is likely that such prior art measured equilibrium constants are associated with heterogeneous oligomer preps and are therefore incorrect, such equilibrium constant values are at best, uninterpretable with regard to whether they represent the entire analyzed oligomer prep. Many such prior art equilibrium constants have been determined for a variety of prior art oligomer preps ranging in N value from 3 to 14 or so. It appears that shorter oligomers tend to be associated with less damage. In addition, certain of the prior art oligomers were enzymatically synthesized and may be associated with only a very small amount of damage. It is possible that the measured equilibrium constants for some of the shorter prior art oligomers are valid and correct. However, prior art did not determine or report on the homogeneity of the entirety of such analyzed short oligomer preps.

Prior art believes and practices that a prior art OM measured Tm value for an oligomer duplex prep represents the Tm value for a homogeneous oligomer duplex molecule population. Further, prior art believes and practices that the oligomer duplex prep equilibrium constant value determined for the Tm represents an equilibrium constant for a homogeneous population of duplex molecules, and therefore represents the correct equilibrium constant value for the analyzed oligomer duplex prep at the Tm. However, as discussed above, such prior art determined equilibrium constant values are highly likely to be incorrect, since the analyzed oligomer duplex preps are highly likely to be heterogeneous. For such a heterogeneous oligomer duplex prep, the more heterogeneous the oligomer duplex prep, the greater the deviation of the prior art determined equilibrium constant value from the true equilibrium constant value for the homogeneous or SF portion of an oligomer duplex prep. This can be illustrated by considering the following hypothetical prior art oligomer duplex OM analysis determination of the Tm and the equilibrium constant.

(a) Equimolar quantities of complementary single strand N oligomer molecules are mixed into the OM analysis solution. The final total single strand oligomer molecule concentration in the OM solution is 4×10⁻⁶M.

(b) Each analyzed complementary oligomer prep has an FH value of 100%, and each oligomer prep used is, unknowingly, composed of 80% SF and 20% FF oligomer molecules. Table 3 indicates that such oligomer preps commonly occur.

(c) To start the OM analysis the OM solution is annealed as discussed earlier so that only the highest quality duplexes will form where possible and the maximum amount of hybridization occurs between the complementary oligomers. Here, the final hybridized total oligomer stringent duplex population will be composed of 80% SF duplexes and 20% FF duplexes and each stringent SF duplex is composed of two essentially undamaged oligomer molecules, and each stringent FF oligomer duplex is composed of two damaged oligomer molecules.

(d) Do the OM analysis and determine the Tm for this oligomer duplex molecule population. At the Tm one half of the oligomer molecules are in a single strand state and one half in a double strand state.

(e) Prior art then determines the analyzed oligomer equilibrium constant for the Tm using the 4×10⁻⁶M total single strand concentration value and the well known relationship, (equilibrium constant value for the analyzed oligomer duplex population)=(4)÷(total single strand oligomer concentration)=(4)÷(4×10⁻⁶M). Here then, the prior art belief is that the analyzed stringent oligomer duplex prep equilibrium constant value is equal to 10⁶ M⁻¹ at the Tm. As discussed this value is incorrect when the analyzed oligomer duplex population is heterogeneous.

(f) Here, for this heterogeneous oligomer prep the Tm value is known to represent the temperature at which one half of the oligomer molecules are in a duplex state and half in a single strand or dissociated state. Therefore, the Tm value for the analyzed oligomer duplex population is accurate.

(g) Here, for this heterogeneous oligomer duplex prep the measured Tm value is not the Tm of the SF oligomer duplex fraction. Because the FF oligomer duplex molecules are far less stable and have t.5d values around 100 times or more smaller than SF oligomer duplex molecule t.5d values, at the Tm the dissociated single strand oligomer molecule population which is present in OM solution is composed of all 20% of the FF oligomer molecules, and the other 30% is composed of dissociated SF oligomer duplex molecules. At the Tm then, ( 30/80) or 0.375 of the total SF oligomer fraction is in the single strand state and 0.625 of the total SF oligomer fraction is in the duplex state.

(h) Since at the prior art oligomer duplex prep OM analysis measured Tm, 0.625 of the SF oligomer molecules are in the duplex form, the true equilibrium constant for the SF duplexes in the OM analysis solution at the measured Tm of the analyzed oligomer prep, is equal to the well known relationship, (2Z)÷(1−Z)² (total SF single strand oligomer concentration in the OM analysis solution), where Z is equal to the fraction of the SF single strand oligomer molecules which are present in a duplex at the measurement temperature, which is here the Tm (16).

(i) Here, Z=0.625 and the total SF single strand oligomer concentration present in the OM analysis solution equals (0.8×4×10⁻⁶M) or 3.2×10⁻⁶M. Therefore, at the measured oligomer prep Tm, (the SF oligomer SF equilibrium constant)=(2×0.625)÷(1−0.625)²(3.2×10⁻⁶M)=2.78×10⁶ M⁻¹.

(j) The ratio of (the true SF equilibrium constant in the OM analysis solution at the measured Tm for the analyzed oligomer prep)÷(the prior art determined equilibrium constant for the analyzed oligomer prep at the Tm), is then equal to (2.78×10⁶ M⁻¹)÷(10⁶ M⁻¹) or 2.78. Thus, the actual equilibrium constant of the SF in the total analyzed oligomer prep is 2.78 fold greater than the prior art practice determined equilibrium constant.

(k) Thus, when a prior art analyzed oligomer duplex prep is associated with significant heterogeneity, the prior art determined equilibrium constant for what the prior art believes is the oligomer duplex fraction of interest at the measured Tm is significantly underestimated, relative to the true SF oligomer duplex of interest equilibrium constant at that temperature. Note that the presence of any degree of heterogeneity associated with a prior art duplex prep will result in such an underestimated equilibrium constant, and the larger the degree of heterogeneity present, the greater the underestimation. This is illustrated in Table 10 using the above described illustrative example with the exception that the degree of heterogeneity associated with the OM analyzed oligomer prep varies from 0 to 49%. Table 10 indicates that the presence of even 5% heterogeneity results in a small but significant underestimate in the prior art determined equilibrium constant.

TABLE 10
Effect of the Heterogeneity of OM Analyzed Oligomer Preps on the
Accuracy of the Prior Art Determined Oligomer Prep Equilibrium Constants

| | | Prior Art Determined | | |
|---|---|---|---|---|
| | | Equilibrium Constant At the | | |
| Fraction In The Total | | Measured Tm For A Total | | Fold Underestimate |
| Analyzed Oligomer | | Analyzed Oligomer | True Equilibrium | of Prior Art |
| Prep | | Concentration of 4 × 10⁻⁶ M. | Constant For | Equilibrium |
| SF | FF | (M⁻¹) | SF (M⁻¹) | Constant for SF |
| 1 | 0 | 10⁶ | 10⁶ | None |
| 0.95 | 0.05 | 10⁶ | 1.23 × 10⁶ | 1.23 |
| 0.90 | 0.1 | 10⁶ | 1.6 × 10⁶ | 1.6 |
| 0.85 | 0.15 | 10⁶ | 2.06 × 10⁶ | 2.06 |
| 0.80 | 0.2 | 10⁶ | 2.77 × 10⁶ | 2.77 |
| 0.75 | 0.25 | 10⁶ | 4.1 × 10⁶ | 4.1 |
| 0.70 | 0.30 | 10⁶ | 6.23 × 10⁶ | 6.23 |
| 0.65 | 0.35 | 10⁶ | 11 × 10⁶ | 11 |
| 0.60 | 0.40 | 10⁶ | 24 × 10⁶ | 24 |
| 0.55 | 0.45 | 10⁶ | 99.8 × 10⁶ | 99.8 |
| 0.53 | 0.47 | 10⁶ | 274 × 10⁶ | 274 |
| 0.51 | 0.49 | 10⁶ | 2450 × 10⁶ | 2450 |

A large prior art literature exists which describes the use of OM analysis determined oligomer duplex Tm values for deriving various thermodynamic parameter values. These thermodynamic parameter values are often used by the prior art for the “rational” design of oligomers for a wide variety of oligomer applications. Such applications include: oligomer primers for PCR and other nucleic acid amplification applications; capture oligomers for microarrays and a variety of different gene expression and pathogen detection applications; oligomers for SNP detection; oligomers for drug use. The validity of these thermodynamic parameter depends of the correct interpretation of the oligomer duplex equilibrium state which actually exists at the Tm. As indicated above, prior art does not know the actual duplex equilibrium state, which actually exists at the prior art measured Tm. Therefore, the accuracy of the thermodynamic parameter values derived from these OM analyses from these OM analyses, is unknown, but highly likely to be incorrect.

Note that even if the prior art belief concerning the OM measured Tm values were valid, the oligomer duplex Tm value has limited utility. For such a valid Tm value, neither the Tm value nor the equilibrium constant derived for the Tm, provides direct quantitative values for the functional characteristics % SF, % FF, FH, kₐ or t.5d (i.e., k_{d}), under the OM analysis conditions or under other oligomer application conditions. In contrast, DK analysis can determine direct quantitative values for the oligomer functional characteristic values FH and t.5d under the analysis conditions and under various oligomer application conditions. In addition, the determination of the quantitative kₐ value for the oligomer can be determined under the analysis conditions or other conditions, during the oligomer FH determination. Overall then, even a valid oligomer Tm value, and the equilibrium constant derived from it, has very limited utility in determining the oligomer functional characteristics under the OM analysis conditions. Further, as discussed earlier an oligomer OM analysis Tm cannot even be determined under many oligomer application conditions.

Effect of an Oligomer's Covalent Association with Chemical and Biological and Other Molecule Groups, Including Ligands and Receptors, on the Oligomer Functional Characteristics.

Many prior art oligomer applications require the production of chemically synthesized and purified oligomer preps whose oligomer molecules are covalently linked to a chemical and/or biological and/or other molecule or groups of molecules. Many of such prior art oligomer prep molecules are covalently linked to chemical and/or biological ligand and/or receptor molecules. Such covalently attached molecule entities are generally covalently associated with the oligomer molecule by a linker molecule, which attaches the group of interest to the oligomer molecule. Such linker molecules can vary greatly in molecular weight and chemical composition. Molecular entities which are commonly covalently attached to oligomer molecules include, but are not limited to, the following. (i) Ligands and haptens such as biotin and Digoxigenin (DIG). (ii) Signal generating molecules such as enzymes and light emitting molecules such as fluors or phosphors. (iii) Receptor type molecules such as antibodies and lectins and streptavidins. (iv) Gold or silver particles and particles containing light emitting molecules. (iv) Chemical functional groups or active groups of many kinds. These include chemically reactive groups used for attachment and chemically reactive groups used to generate a chemically induced signal such as an electrical signal. (v) Other nucleic acid oligomers which are not naturally associated with the oligomer of interest. Here, oligomers dAₙ of a defined length is often associated with an oligomer of interest during oligomer synthesis. No non-nucleic acid linker group is required for such an association. Herein, an oligomer of interest, which is associated with a desired group, such as those described above is termed a functionalized oligomer molecule or is termed an F-oligomer.

Prior art sometimes evaluates the effect of the association of the desired group with the oligomer on the F-oligomer functional characteristics. For such evaluations prior art does not determine or take into consideration the heterogeneity of the oligomer prep of interest. Such prior art evaluations of F-oligomers are generally done by OM analysis determination of an F-oligomer duplex prep Tm, under OM analysis conditions which are not the F-oligomer analysis application conditions. As discussed earlier, such an analysis is limited in its ability to provide meaningful information concerning the actual F-oligomer functional characteristics.

The above described determination of the % SF, % FF, FH, FD, kₐ, and t.5d values can be used to evaluate an F-oligomer preps functional characteristics and thereby obtaining F-oligomer functional characteristic results which are relative to such prior art obtained F-oligomer functional characteristic results, significantly improved in correctness and utility. The generation of such improved F-oligomer functional characteristic results is a practice of the present invention.

Table 11 presents the measured functional characteristics of a variety of F-oligomer preps which are associated with one or more of the groups, biotin, NH₂, dA₂₅, dA₃₅, thiol, and fluor. Note that while the analysis conditions used in Table 11 for the various F-oligomers examined are generally not the application analysis conditions, such functional characterization results can be readily obtained for most oligomer application conditions.

TABLE 11
The Effect of the Attachment of Ligands or Fluors or Functional
Groups to an Oligomer on the Functional Homogeneity and Functional
Characteristics of the Oligomer

| | | | | HDB Stringent Values Determined For | | | |
|---|---|---|---|---|---|---|---|
| | | Complementary | | | | | SF t.5d |
| P³² Oligomer | | Oligomer | Type of Group Added | FH | % FF | % SF | (m) |
| 1) | (a) dT₃₅ | dA₃₅ | None | 99.6% | 18 | 82 | 15.8 (50° C.) |
| | dT₃₅.NH₂ | dA₃₅ | Linker —NH₂ on 3′ end | 99.5% | 22 | 78 | 17.1 (50° C.) |
| | (b) dT₃₅ | dA₃₅ | None | 99.6% | 18 | 82 | 15.8 (50° C.) |
| | dT₃₅.NH₂ | dA₃₅ | Linker —NH₂ on 3′ end | 84.6% | 52 | 48 | 17.2 (50° C.) |
| 2) | dT₃₅.NH₂ | dA₃₅ | Linker —NH₂ on 3′ end | 99% | 35 | 65 | 17.5 (50° C.) |
| 3) | (a) dA₃₅.Biotin | dT₃₅.NH₂ | Linker —B on 3′ end | 97.9% | 42 | 58 | 14 (50° C.) |
| | (B) | | dA₃₅ | | | | |
| | (b) | dT₃₅.NH₂ | Linker NH₂ on 3′ end dT₃₅ | 98.9% | 36 | 64 | 14.3 (50° C.) |
| | dA₃₅.Biotin | | Linker —B on 3′ end | | | | |
| | | | dA₃₅ | | | | |
| | | | Linker NH₂ on 3′ end dT₃₅ | | | | |
| 4) | BS N = 24 | BS.A₂₅.NH₂ | dA₂₅ attached to 3′ end | 97.4% | 14 | 86 | 28 m (53° C.) |
| | | BS is PM | of BS and 3′ linker.NH₂ | | | | |
| | | N = 49 | attached to 3′ end of | | | | |
| | | | dA₂₅ | | | | |
| 5) | (a) BS.A₃₅.F1 | BS N = 47 | Fluor group on 3′ end | 76% | 66 | 34 | 36 (52° C.) |
| | N = 64 | PM to BS | of chimeric RNA.DNA | | | | |
| | Chimeric RNA, | | oligomer | | | | |
| | DNA | | | | | | |
| | (b) BS.A₃₅.F1 | BS.T₃₅ PM | Fluor group on 3′ end | 70.7% | 70 | 30 | 75 (52° C.) |
| | N = 64 | N = 64 | of DNA oligomer | | | | |
| | DNA only | | | | | | |
| 6) | BS.S | BS N = 89 | Thio molecule on 3′ end | 63.6% | 56 | 44 | 95 (47° C.) |
| | N = 29 DNA | DNA | | | | | |
| | | Contains PM | | | | | |
| | | for N = 29 | | | | | |
| | | BS.S | | | | | |
| | | Internally | | | | | |

Alternative Methods for Determining Oligomer Functional Homogeneity and Functional Characteristics.

Thus far the discussion of the determination of improved information concerning the functional homogeneity and improved functional characteristics for an oligomer prep and other aspects of the practice of the present invention have been in terms of a presently preferred analysis system utilizing P³² oligomer, and an HA analysis method, and the use of temperature to induce and control oligomer duplex dissociation. However, multiple alternative analysis systems exist which can be utilized to provide improved information concerning the functional homogeneity and improved functional characteristics for an oligomer prep and practice the various aspects of the present invention. This is discussed below.

Oligomers can be labeled with a wide variety of radioactive or non-radioactive signal generation molecules besides P³². Such commonly used radioactive molecules include, but are not limited to, P³³, I¹²⁵, S³⁵, H³, and C¹⁴. Methods for the direct and indirect labeling of oligomer and other nucleic acid molecules in general are well known and commonly used. Such commonly used non-radioactive molecules include, but are not limited to, a wide variety of enzymes, standard fluorescent molecules and fluorescence quenching molecules, and time resolved related fluorescent molecules and phosphorescent molecules, a wide variety of chemical and biological luminescent molecules, and various metal and non-metal nanoparticles. Such fluorescent and/or chemiluminescent molecules as well as many chromogenic molecules serve as enzyme substrates and direct or indirect signal label which can be used to accurately quantitate the absolute amount of labeled oligomer present in a sample, or which can be used to accurately quantitate the relative amounts of labeled duplex oligomer molecules and labeled non-hybridized or single strand oligomer molecules in a sample, and can be used to practice the present invention and determine the oligomer functional homogeneity and functional characteristics.

While each of the above-mentioned alternative signal molecules mentioned can be used to practice the present invention, some have greater ease of use and practical utility and analytical precision than others. Generally the radioactive signal labels provide the greatest analytical utility. Generally however, the use of radioactivity is discouraged for practical reasons. The most commonly used prior art alternative signal molecule to radioactivity is fluorescence molecules by far, with chemiluminescence a distant second. Prior art oligomer and nucleic acid in general fluorescence labeling practice, generally prefers the direct covalent attachment of one or more fluor molecules to the oligomer or nucleic acid molecule. Prior art oligomer practice often designs and produces fluorescent labeled oligomer molecules with one or more fluor molecules attached to an intended location in the oligomer. Prior art also often designs fluor labeled oligomer molecules which contain a precisely positioned fluor molecule, and in addition a precisely positioned fluorescence quenching molecule. Such an arrangement can result in differences in fluorescent signal amount for duplex and single strand oligomer molecules, and depending on the design the signal associated with an oligomer molecule in a single strand state may be significantly larger or smaller than the fluorescent signal associated with the same oligomer which is duplexed. Many different types of such fluorescent oligomers and their use for detecting and quantitating the extent of oligomer hybridization are present in the prior art. Prior art widely used molecular beacon oligomers and FRET oligomers are examples of such fluor labeled oligomers. TaqMan or 5′ nuclease assays also utilize such fluor labeled oligomers. Other prior art methods such as the Syber green method do not require the covalent attachment of a dye to the oligomer molecules. For such methods the free in solution dye molecules interact with unlabeled oligomer duplexes to give a greater fluorescent signal than the interaction of the dye with unlabeled oligomer single strand. A variety of such prior art non-covalent dye-oligomer duplex methods are available. One skilled in the art will be aware of the many different methods for using fluorescent signal molecules to detect and quantitate single strand and duplex oligomer and other nucleic acid molecules.

A wide variety of alternative prior art methods exist for detecting and quantitating the extent of hybridization of a single strand labeled or unlabeled oligomer or other nucleic acids, and quantitatively determining the absolute or relative amounts of duplex and single strand oligomer in a sample of interest. Such methods include, but are not limited to, the following. (a) Well known optical methods for determining oligomer hybridization and dissociation. The measured signal can be the absorbance of the oligomer nucleic acid itself or a fluorescent signal from dye molecules, which are covalently or non-covalently attached to the oligomer of interest. (b) Methods which rely on size or ionic charge differences between the oligomer duplex form and the single strand oligomer form in order to separate, detect, and quantitate the amount of duplex and single strand oligomer in a sample. Such methods include, but are not limited to: various gel electrophoresis methods including capillary electrophoresis; various non-electrophoresis molecular size exclusion methods such as column chromatography; various methods using mass spectroscopy; various sedimentation methods. (c) Methods which rely on the immobilization of one nucleic acid complementary strand in order to separate, detect, and quantitate the amount of duplex and single strand oligomer in a sample. Such methods include, but are not limited to, various methods such as northern blots and dot blots, microarrays, and bead based systems. (d) Various methods which rely on the specificity of a nuclease for duplex or single strand molecules in order to detect and quantitate the amount of duplex and single strand oligomer in a sample. Such methods include the S-1 nuclease method, other nuclease protection methods, and specific nuclease based mismatch detection methods. (e) Methods which rely on the use of affinity capture columns to separate, detect, and quantitate the amount of duplex and single strand oligomer molecules in a sample.

A variety of prior art methods exist which can be used for inducing or preventing and controlling the dissociation of oligomer and other nucleic acid duplexes at a particular temperature. Such methods include, but are not limited to, the following. (a) The concentration of certain organic and inorganic chemical compounds or polymers in the oligomer duplex analysis solution. Such chemical compounds include formamide, DMSO, trichloroacetate salts, different mono- and di-valent inorganic salts, formaldehyde and similar compounds, alcohols, phenols, urea, chaotropic salts, dye and other intercalating compounds, and reagents, which react specifically with mismatched or unpaired bases. (b) The pH of the duplex analysis solution. (c) Various combinations of a and b and temperature.

Any method which allows the detection and quantitation of the absolute and/or relative amount of the duplex and single strand forms of the oligomer of interest in a sample can be used for obtaining improved information concerning the oligomer functional homogeneity and functional characteristics and for practicing the present invention. Many different prior art methods, including those listed above, can be used for the effective practice of the present invention. While each of such prior art methods can be used for the practice of the invention, each has a different combination of analysis strengths and weaknesses. Practically this means that while a particular prior art method can be used to determine all aspects of an oligomer's functional homogeneity and functional characteristics, certain of the aspects are readily determined and others are possible to determine but are far less practical to determine. One of skill in the art will be able to determine or know the strengths and weaknesses of each method and use the method appropriately for the practice of the invention.

It is highly likely that a variety of fluorescence based methods for the detection and quantitation of duplex and single strand oligomer will be heavily used for the practice of the invention in the future, as will capillary electrophoresis based methods.

A representative example of an existing fluorescence based detection system which can readily be modified to practice the present invention is the use of a temperature controlled real time PCR cycling system which is configured to detect fluorescent signal from the incubated sample. In contrast to a PCR analysis, a sample to be analyzed would be placed into the PCR cycler at a temperature where duplex dissociation is negligible, and then the PCR cycler temperature would be rapidly raised to the desired DK analysis temperature. At this desired constant PCR cycler temperature the DK analysis is done, and the duplex dissociation can be quantitated and monitored by the change in fluorescent signal which occurs as the duplex molecules are converted to single strand molecules. Depending on the fluorescent system chosen, the fluorescence may decrease or increase as the duplex molecules are converted to single strand molecules. As an example, when using fluorescent molecular beacons to monitor the dissociation, the fluorescent signal will decrease as the duplex is converted to single strand (36, 37). In contrast, for other fluorescent systems including other fluorescent energy transfer systems, the fluorescent signal increases as the duplex dissociation proceeds. Existing real-time PCR platform systems such as the commercially available ABI, Roche, Stratagene, BioRad, MJ Research, and others (37), can be modified for DK analysis such systems can also be used to monitor hybridization. Existing fluorescent microtiter plate readers can also be adapted for the practice of the invention and DK analysis.

Producing Improved Oligomer Functional Homogeneity and Functional Characteristic Results for a Variety of Different Purposes: Examples of Various Practices of the Present Invention.

Prior art oligomer preparations are intended for use in a particular oligomer application. The acceptable and optimal functional effectiveness of the oligomer application are directly influenced by the actual functional homogeneity and functional characteristics of the oligomers utilized in the oligomer application. Designing the oligomer used, and evaluating the actual efficacy of an oligomer prep in the oligomer application, and the degree of effectiveness of the oligomer application for its intended use, requires accurate, and as extensive as possible knowledge of the actual functional homogeneity and functional characteristics of an oligomer prep. As discussed above, pertinent functional homogeneity and functional characteristics, which are associated with an oligomer prep and its use in an oligomer application, include the following. (i) The oligomer hybridization kinetic or association constant kₐ. (ii) The oligomer FH and/or FD value. (iii) The functional homogeneity or heterogeneity of the oligomer prep. (iv) The stringent % SF and % FF values associated with the oligomer prep. (v) The t.5d and/or dissociation constant k_{d} value for the oligomer prep SF and FF components. (vi) The equilibrium constant associated with the oligomer prep SF and FF. The equilibrium constant for an oligomer duplex can be determined from the duplex's association and dissociation constants. Knowledge of such oligomer functional homogeneity and functional characteristic values for a standard or model system has great utility and obtaining such values is a practice of the invention. For a particular oligomer application it is preferable to have, or also have, knowledge of such oligomer functional homogeneity and functional characteristic values for the oligomer application conditions. Obtaining such values is a practice of the present invention, and the use of such improved results in the oligomer application is a further practice of the invention, which will provide further improved results for the oligomer application.

Prior art does not determine or know or consider such extensive knowledge for an oligomer under standard or model conditions or the oligomer application conditions. Further, prior art does not determine or know or consider the relationship between the functional homogeneity and functional characteristic values associated with such extensive knowledge, and the effectiveness of the use of the oligomer in the oligomer application. Further, a significant portion of the limited knowledge which prior art has concerning the oligomer functional homogeneity and functional characteristic values, cannot be known to be correct and is very likely to be erroneous. Because of the incomplete and limited and uncertain nature of prior art determined knowledge concerning the oligomer functional homogeneity and functional characteristic values, evaluating the relationship between the prior art known oligomer functional homogeneity and functional characteristic values and the effectiveness of the use of the oligomer on the oligomer application effectiveness, is problematic at best. This occurs because absent accurate and extensive knowledge of the oligomer functional homogeneity and functional characteristic values, there is no way to correlate the oligomer functional homogeneity and functional characteristics to the effectiveness of the oligomer application for its intended use. For example, a PCR assay which is optimized using a heterogeneous oligomer primer prep which has a particular degree of oligomer damage associated with it, may have an apparently degraded PCR assay performance when used in conjunction with a second lot of the same primer, which second lot is by prior art standards of higher quality that the development lot.

Relative to the prior art situation, the practice of invention provides improved oligomer prep functional homogeneity and functional characteristic results and information, and improved oligomer applications, and improved oligomer application results. Such present invention improved aspects include, but are not limited to, the following. (a) Much more extensive and accurate knowledge concerning the oligomer functional homogeneity and the oligomer functional characteristic values. (b) Much more extensive and accurate knowledge concerning the relationship between the oligomer functional homogeneity and functional characteristic values and the effectiveness of the oligomer application for its intended use. (c) Much more extensive and accurate knowledge concerning the relationship between the intended oligomer prep functional homogeneity and functional characteristic values and the intended effectiveness of the oligomer application. (d) Improved oligomer application design and performance. (e) Improved oligomer application results. (f) Improvement for any oligomer application product and/or process and/or result, product. (g) Improvement for any process, product, or any other application, which uses improved oligomer application results. (h) Improvement for any process or product or reagent or protocol which is related to oligomer synthesis and production.

The practice of the invention provides much more extensive and accurate knowledge concerning an oligomer prep's functional homogeneity and functional characteristics and effectiveness in an oligomer application relative to such prior art knowledge. Because of this the present invention provides oligomer functional homogeneity and functional characteristic results, and oligomer applications and oligomer application results which are, relative to prior art oligomer functional homogeneity and functional characteristic results, oligomer applications, and oligomer application results, significantly improved. The production and use of such improved results, and information concerning the functional homogeneity and functional characteristics of an oligomer prep and/or the production or use of such improved oligomer applications, and/or the production and use of such improved oligomer application results for other purposes of any kind, constitute practices of the present invention. In other words, any use of improved oligomer functional homogeneity or functional characteristic results, improved oligomer applications results, or improved oligomer applications to produce further improved results, information, or products or product results, is a practice of the invention.

Specific examples of the practice of the present invention are presented below. Each example will specify a specific purpose for the practice of the invention.

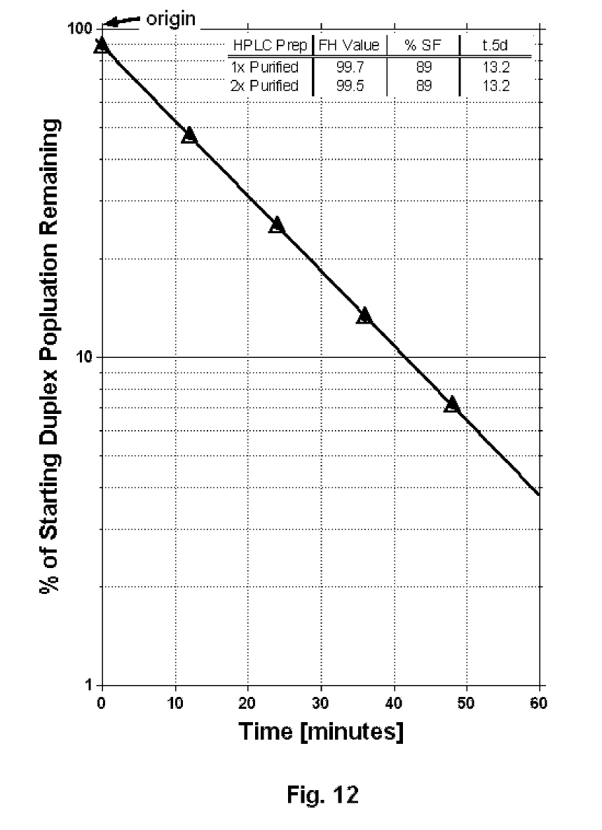

Example One's purpose is to utilize the practice of the invention to obtain improved information concerning whether a second HPLC purification increases the quality of a purified N oligomer prep. This was done as follows. (a) Chemically synthesize a dT₃₅ oligomer preparation. (b) Purify the entirety of the dT₃₅ oligomer synthesis prep on HPLC and save the appropriate purified fraction. This fraction is termed the HPLC 1 fraction or the H1 fraction. (c) Half of the purified H1 dT₃₅ fraction was HPLC purified a second time using the same HPLC method used for the first purification. This second HPLC purified dT₃₅ prep is termed the H2 dT₃₅ prep. (d) Separate aliquots of the H1 and H2 dT₃₅ preps were separately P³² labeled and purified. (e) Each P³² dT₃₅ prep was separately mixed with an excess of the same lot of UC dA₃₅ oligomer and hybridized by annealing as described earlier to form stringent P³² dT₃₅·dA₃₅ duplexes. (f) The measured FH values were 99.7% and 99.5% respectively for the H1 and H2 P³² dT₃₅ oligomer preps. (g) A DK analysis profile was then obtained for each H1 and H2 P³² duplex population. These profiles are presented in FIG. 12. The DK analysis profiles show that the H1 and H2 P³² dT₃₅ oligomer preps have essentially identical FH, stringent % SF, and t.5d functional characteristic values. This indicates that a second HPLC purification step did not result in a higher % SF value, and that a second HPLC purification step did not improve the dT₃₅ oligomer prep, and was, therefore, not necessary.

Example Two's purpose is to determine if scaling up the oligomer amount synthesized affects the quality of a N=55 biological sequence DNA oligomer used for a diagnostic assay application. The oligomer quality parameters evaluated were the oligomer functional characteristics FH, % SF, and t.5d.

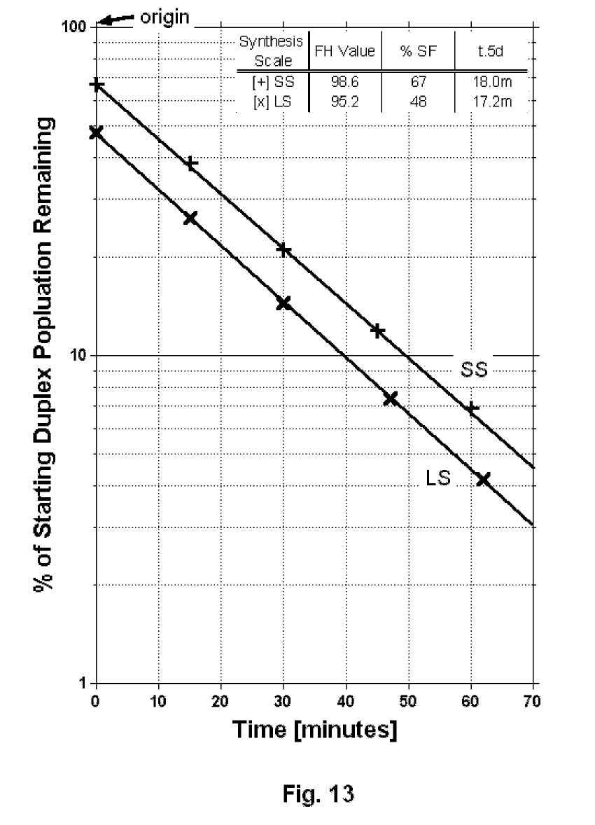

This practice of the invention was done as follows. (a) The same oligomer was produced by a small research scale DNA synthesizer and a much larger scale DNA synthesizer. Each oligomer prep was separately purified to produce an N oligomer prep. Here, the small scale oligomer is termed the SS oligomer and the large scale oligomer prep is termed the LS oligomer prep. (b) Each of the oligomer preps were separately P³² labeled and purified. (c) Each P³² oligomer prep was separately and stringently annealed to the same lot of perfectly complementary UC oligomer. (d) The measured FH values for the P³² oligomers were SS=98.6% and LS=95.2%. (e) A DK analysis profile was then obtained for each stringent P³² oligomer duplex prep. These profiles are presented in FIG. 13. The DK profiles show that the SS and LS t.5d values are essentially the same but that the amount of undamaged SF oligomer was significantly greater for the SS N oligomer prep. This indicates that the quality of the SS N oligomer prep molecules is significantly better than the quality of the LS oligomer prep molecules. Note that according to the prior art characterization quality specs used by the SS and LS oligomer producer the quality of the SS and LS oligomers was the same.

Example Three's purpose is to utilize the practice of the invention to obtain improved information as to whether exposure to standard concentrated acetic acid (80%) under conditions routinely used for oligomer synthesis, significantly damages the SF of the N oligomer prep. The effect of the concentrated 80% acetic acid on the stability of a purified P³² dT₃₅ N oligomer was done as follows. (a) Produce a dT₃₅ P³² N oligomer prep. (b) With an aliquot of the P³² dT₃₅ produce a stringent P³² dT₃₅·dA₃₅ duplex prep in HDB as described. (c) Obtain a DK profile for the stringent P³² dT₃₅·dA₃₅ duplex prep. The P³² dT₃₅ oligomer prep had measured values for the FH of 98.9%, the stringent duplex % SF of 72% and an SF t.5d value of 13.4 m. (c) A separate aliquot of the P³² dT₃₅ oligomer was diluted into concentrated acetic acid and incubated at 25° C. for 651 hours. The final acetic acid concentration was 80%, a concentration often used during prior art oligomer synthesis. Essentially all of the treated P³² dT₃₅ oligomer was then recovered by ethanol precipitation in the presence of glycogen. (d) The treated P³² dT₃₅ oligomer was then used to produce a stringent P³² dT₃₅·dA₃₅ duplex in the same manner as the control. A DK profile was then obtained for the stringent P³² dT₃₅·dA₃₅ duplex prep. The treated P³² dT₃₅ oligomer prep had measured values for the FH of 97.4%, the stringent duplex % SF of 73% and an SF t.5d of 11.6 m. Note that a different batch of HDB DK analysis solution was used for determining the 50° C. HDB t.5d value for the control and treated sample. (e) The control and 651 hour 80% acetic acid P³² dT₃₅ samples showed little or no difference in the measured FH values, the stringent duplex % SF values, or the t.5d values. This indicates that exposure to the concentrated 80% acetic acid for over 27 days at 25° C. had little effect on the functional characteristics or integrity of the treated SF oligomer dT35 molecules. Such results indicate that the damage associated with the P³² dT₃₅ FF oligomers is not caused by exposure to the low pH high concentration acetic acid treatment during the dT₃₅ oligomer synthesis and processing.

Example Four's purpose is to utilize the practice of the invention to obtain improved information as to whether exposure to standard concentrated ammonium hydroxide (29%) under conditions commonly used for oligomer synthesis, significantly damages the SF of an N oligomer prep. This was done as follows by utilizing a dT₃₄·A₁ N oligomer prep, with A at position 5. (a) Prepare a dT₃₄·A₁ N oligomer prep. (b) With an aliquot of this P³² oligomer produce a stringent dT₃₄·A₁·(dA₃₅) duplex prep in HDB. (c) Obtain a 47° C. HDB DK profile for the stringent P³² oligomer duplex prep. The P³² dT₃₅ oligomer prep had measured values for the FH of 97.8%, the stringent duplex % SF of 80%, and the HDB 47° C. t.5d value of 15.8 m. (d) A separate aliquot of the P³² dT₃₄·A₁ oligomer prep was diluted into concentrated ammonium hydroxide and incubated at 25° C. for 717 hours (29.9 days). The final ammonium hydroxide concentration was 29%, a concentration commonly used during the prior art oligomer synthesis process. Essentially all of the treated P³² oligomer was then recovered by ethanol precipitation in the presence of glycogen. (d) The treated P³² dT₃₄·A₁ polymer was then used to produce a stringent P³² dT₃₄·A₁·(dA₃₅) duplex prep. A DK profile was then obtained for the P³² oligomer stringent duplex prep. The treated P³² dT₃₄·A₁ oligomer prep had measured values for the FH of 95.7%, the stringent duplex % SF of 78% and a 47° C. HDB t.5d of 14 m. (e) The control and 717 hour ammonium hydroxide treated dT₃₄·A₁ samples show little or not difference in the measured FH values, the stringent duplex % SF values, or the t.5d values. This indicates that exposure to the concentrated 29% ammonium hydroxide for about 30 days had little effect on the integrity or functional characteristics of the SF oligomer. Such results indicate that the damage associated with P³² dT34·A₁, FF oligomers is not caused by exposure to the high pH ammonium hydroxide treatment during oligomer synthesis.

Examples one thru four represent practices of the invention whose purpose was to evaluate the effect of some aspect of the synthesis chemistry or chemicals, or process, or instrument, on the oligomer quality. Similar type evaluations can be done for every chemical used in the oligomer synthesis and every step of the synthesis process and every step of the post-synthesis treatment and storage and use process.

Example Five's purpose is to utilize the various aspects of the practice of the present invention in order to generate improved functional information values which can be used for improved rational evaluation of existing oligomer applications and the improved rational design and development and optimization and quality control and production and use, of oligomer applications in general. Examples of such oligomer functional characterization values are presented here in FIGS. 4 thru 12, and FIGS. 14 and 15, as well as Tables 1 thru 11. These examples are only a very small fraction of the pertinent such values, which can be produced thru the practice of the present invention.

Example Six's purpose is to practice the present invention by utilizing the information and results produced by some version of example Five in order to obtain oligomer applications results which are improved. An example of this is the improved OM measured oligomer duplex equilibrium constants, which can be obtained through the consideration of improved oligomer functional characteristic result. This is but one of a very large number of oligomer application results which can be improved. The performance of and the results from the vast majority of prior art oligomer applications of all kinds can potentially be improved by the practice of the invention.

Example Seven's purpose is the practice of the invention through the use of improved oligomer applications results in order to produce improved results for an application which utilizes in some way improved oligomer application results of one sort or another. An example of this is the use of multiple improved gene expression or SNP detection oligomer application results, in an application which utilizes such results to obtain an improved desired medical or basic research result or analysis or product. Such applications include, but are not limited to, the following. (a) Systems biology analyses and results. (b) Algorithms for disease prognosis with and without treatment. (c) Data mining and clustering analyses of all kinds. Another example of this is the use of multiple improved OM derived thermodynamic results in applications, which utilize such results for predicting nucleic acid structure and function for various purposes. These cited examples are only two of a large number of situations where improved oligomer application results are utilized in an application to provide improved results for that application.

The Practice of the Invention for the Purpose of Detecting Single Nucleotide Polymorphisms (SNP) in DNA or RNA.

RNA and/or DNA oligomers and/or modified RNA and DNA oligomers play a central role in the prior art methods which are used for the detection of single nucleotide polymorphisms (SNP) or mutations in biological and other nucleic acids. The practice of the present invention allows the production of oligomer and oligomer-target duplex functional homogeneity and functional characteristic information and quantitative values which are improved, relative to prior art produced functional homogeneity and functional characteristic information and quantitative values. This oligomer and oligomer-target duplex improved information and quantitative values can be used to produce single nucleotide polymorphism (SNP) or mutation detection assays and assay results which are improved in rationality of design and/or discrimination and/or simplicity and/or utility. Such invention improved SNP and mutation detection assays will produce SNP and mutation detection qualitative and quantitative results which are improved relative to prior art produced SNP and mutation detection results. In other words, the practice of the invention can be utilized to produce SNP and mutation detection assays and assay results which are improved relative to prior art SNP and mutation detection assay results.

Prior art SNP detection assays are generally either hybridization based or nucleic acid sequencing based assays. A variety of different types of hybridization based SNP assays are utilized by the prior art. Essentially all of these different types of hybridization based SNP assays and the assay results produced by them can be improved by the practice of the invention.

As indicated earlier it is highly likely that the N oligomer molecule population which is present in almost all prior art oligomer preparations is significantly heterogeneous, and contains significant nucleotide sequence damage. The population of damaged N oligomer molecules consists of N oligomer molecules which have the same nucleotide length, and which are similar but not identical in nucleotide sequence. The evidence indicates that the extent of nucleotide sequence damage associated with an average damaged N oligomer molecule is equivalent to the presence of 1-2 strongly destabilizing mutations. In effect this damaged N oligomer molecule population represents a heterogeneous population of N oligomer molecules, all of which contain mutations or damage, and for different oligomer molecules in the damaged population the mutation or damage occurs at different nucleotide sequence positions. This results in a situation where the damaged or mutated N oligomer molecule population is composed of oligomer molecules which have the same nucleotide length and are similar but not identical in nucleotide sequence. Thus an N oligomer molecule prep which is used to detect a specific mutation in a prior art SNP assay, contains a significant, heterogeneous, population of N oligomers which are also mutated or damaged. Further, prior art believes that the oligomer N molecule population contains little significant nucleotide sequence damage, and does not determine or know the quantitative functional homogeneity or functional characteristic values, or the extent of nucleotide sequence damage, associated with the purified N oligomer damaged and heterogeneous fraction.

A purified N oligomer prep which is used in a prior art SNP assay to detect mutations is then, likely to be composed of a significant and homogeneous fraction of N oligomer molecules which have no detectable nucleotide sequence damage, and a significant and heterogeneous fraction of N oligomer molecules which are associated with significant nucleotide sequence damage or mutation. Therefore, prior art SNP assays which utilize purified N oligomers contain a significant fraction of damaged N oligomer molecules which are associated with heterogeneous damage or mutations, and which affect the stability of the hybridized N oligomer-target duplex molecules to different extents. The stability of some of the hybridized damaged N oligomer-target duplexes formed by the N oligomer prep will be affected to the same, or nearly the same, extent as an N oligomer-target duplex containing a centrally located, strongly destabilizing mutation, while the stability of other hybridized damaged N oligomer-target duplexes will be affected to a greater or lesser extent, and the maximum stability will be associated with hybridized undamaged N oligomer-target duplexes. This situation greatly complicates the development and interpretation of prior art SNP assays, and results in prior art hybridization based SNP assays which are significantly sub-optimal in assay rationale, assay design, and assay performance. Knowledge of a purified N oligomer prep quantitative functional homogeneity and functional characteristic values, and the pattern and extent of nucleotide sequence damage associated with the purified N oligomer prep, is essential to optimize and improve prior art hybridization based SNP assays.

Prior art hybridization based SNP assays often utilize crude unpurified oligomer preps for the assay. Such crude oligomer preps almost always contain a significant fraction of truncated or N−X oligomer molecules. It is not unusual for a crude oligo prep to consist of half or more truncated or N−X oligomers. Very often each N−X molecule in a crude prep also contains a deleted base. A deleted base has an effect on hybridized oligomer-target duplex stability which is similar to a strongly destabilizing mutation. In addition, the presence of a N−X oligomer in an otherwise perfectly base pair matched hybridized-oligomer-target duplex, also lowers the stability of a hybridized oligomer-target duplex. Thus, the N−X oligomer molecule population in a crude oligomer prep is composed of a heterogeneous population of short and long N−X molecules, and each of these short and long N−X oligomer molecules may be associated with a deletion. A crude oligomer prep is also highly likely to contain a significant fraction of N oligomer molecules which are heterogeneous in nucleotide sequence. As discussed earlier, these heterogeneous N oligomer molecule populations consist of oligomer molecules which are damaged or mutated in nucleotide sequence. A crude oligomer prep is also likely to contain a significant fraction of N oligomer molecules which have the same nucleotide length and the same nucleotide sequence. This homogeneous population of purified N oligomer molecules will form hybridized N oligomer-target duplexes which do not contain mismatched base pairs and therefore have the maximum N oligomer-target duplex stability.

A crude oligomer prep which is used in a prior art SNP assay to detect mutations is then highly likely to be composed of: a significant and homogeneous fraction of N oligomer molecules which apparently are not associated with nucleotide sequence damage; a significant and heterogeneous fraction of N oligomer molecules which are associated with significant nucleotide sequence damage; a significant and heterogeneous fraction of truncated or N−X oligomer molecules associated with deletions which occur at different positions in different N−X molecules; and N+X oligomer molecules may be present. Prior art SNP assays which utilize unpurified or crude oligomers then, contain a very significant fraction of oligomers which in effect contain heterogeneous mutations which affects the stability of a hybridized oligomer-target duplex to different extents. The stability of some of the hybridized oligomer-target duplexes present in a hybridized crude oligomer-target duplex preparation will be affected to the same extent as a centrally located, strongly destabilizing mutation, while the stability of other oligomer-target duplexes will be affected to a greater extent than this, and the stability of other hybridized oligomer-target duplexes will be affected to a lesser extent than this, while hybridized undamaged N oligomer-target duplexes have the greatest stability. This situation greatly complicates the development and interpretation of prior art SNP assays, and results in prior art hybridization based SNP assays which are significantly sub-optimal in assay rationale, assay design, and assay performance. Knowledge of the crude oligomer preps quantitative functional homogeneity and functional characteristic values and the pattern and extent of nucleotide sequence damage associated with the heterogeneous crude oligomer prep, is essential to improve prior art hybridization SNP assays.

Certain DNA sequencing based SNP detection methods utilize one or more oligomers. These methods can also be improved by the practice of the invention. The rationale for these invention improvements is generally the same as those just discussed for improving hybridization based SNP assays and results by the practice of the invention.

Application of Basic Invention Rationale for Determining the Functional Homogeneity and Functional Characteristics of Triplex or Quadriplex Nucleic Acid Molecules.

Triplex and quadriplex nucleic acid complexes are well known. The basic rationales, methods, and approaches used to practice the invention for single and duplex nucleic acid molecules can be utilized to determine the functional homogeneity and functional characteristic values for: triplex nucleic acid molecules which consist of 3 separate nucleic acid molecules, and quadriplex nucleic acid molecules which consist of 4 separate nucleic acid molecules, as well as the individual nucleic acid molecules which comprise such triplex and quadriplex molecules. The determination of such triplex and quadriplex associated quantitative functional homogeneity and functional characteristic values produces improved values, relative to prior art values, and is a practice of the invention. Overall then, the basic practice of the invention is applicable to situations where dissociable nucleic acid complexes are formed in solution or on a solid surface.

Application of Basic Invention Rationale for Determining the Functional Homogeneity and Functional Characteristics of Nucleic Acid Duplex, Triplex, or Quadriplex Molecules which are in the Presence of Other Small or Large Biological and/or Non-Biological Molecules.

The basic rationales, methods, and approaches which are used to practice the invention for single and duplex nucleic acid molecules can be utilized to determine the effect of the presence of small and large biological and/or non-biological molecules on the quantitative functional homogeneity and functional characteristic values of duplex, triplex or quadriplex nucleic acid complexes. The determination of these values produces improved values relative to the prior art values, and is a practice of the invention.

The Practice of the Invention for the Characterization of Oligomers Which are Immobilized on a Surface.

Many prior art oligomer applications involve oligomer molecules, which are immobilized on a solid surface. Such surface oligomers are either synthesized on the surface of the application device, or are synthesized by standard prior art oligomer synthesis methods and then placed on the surface of the application device. For the synthesized in place immobilized oligomer application the immobilized oligomer molecule population represents a crude oligomer prep, and contains N−X oligomers and can also contain N+X oligomer molecules. Generally the coupling efficiency of in place synthesis is much lower than that of standard oligomer synthesis, and the in place synthesized crude oligomer prep contains far more N−X oligomer molecules than does the standard oligomer synthesis crude oligomer prep. Because of this, the quality of the synthesized in place immobilized oligomer is much lower than the quality of the immobilized oligomer molecules, which were synthesized by standard methods and then immobilized. Herein, oligomers synthesized in place are termed in situ synthesized oligomers or ISS oligomers or oligomer preps, while oligomers synthesized by standard methods and then immobilized on a surface is termed separately synthesized oligomers or SSS oligomers or oligomer preps.

ISS crude oligomer preps with an intended N of 25 or so often contain 90% or so N−X oligomer molecules. Such ISS crude oligomer preps cannot be further purified to eliminate or reduce the large amount of immobilized N−1 oligomer and its associated nucleotide sequence damage from the ISS crude oligomer prep. In contrast, SSS crude oligomer preps can be and often are further purified to greatly reduce the amount of N−1 oligomer present in the purified prep. SSS crude oligomer preps with an intended N of 25 virtually always contain far less N−1 oligomer than a comparable ISS crude oligomer prep. A typical SSS crude oligomer prep with an intended N=25 usually contains about 30% or so N−1 oligomer. In addition, a crude SSS oligomer prep can be further purified so that the purified prep contains 5-10% or less N−1 oligomer. However, SSS crude oligomer preps are rarely further purified before surface immobilization. Even so, the quality of the immobilized SSS oligomers is generally much higher than the immobilized ISS oligomers. For such prior art immobilized ISS and SSS crude oligomer molecule situations, it is known that the immobilized oligomers are heterogeneous, but it is not known how heterogeneous the immobilized oligomer molecule populations are, or the quantitative amounts of each N−X molecule type which is in each oligomer molecule population. Absent further knowledge then, the prior art immobilized oligomer molecule populations are unknown with regard to oligomer homogeneity and degree of homogeneity and the amount and type of each different oligomer molecule type in the immobilized oligomer molecule population.

For the SSS crude oligomer prep, the homogeneity, the degree of homogeneity, and a measure of the amount of different oligomer types present, and a measure of the oligomer functional characteristic values, can be determined as described earlier by the practice of the invention. However, the immobilization of the crude oligomer prep molecules, can and probably does change all these factors and values significantly. Further, the existing evidence indicates that the surface immobilization of a perfectly functionally homogeneous oligomer molecule population would result in an immobilized oligomer molecule population which is functionally significantly heterogeneous. This probably occurs because of the many potentially different ways a single oligomer molecule can interact with the surface and become immobilized, and the microheterogeneity of the surface oligomer immobilization sites. There would be similar concerns for a perfectly homogeneous population of ISS oligomer molecules on the surface. Such surface functional heterogeneity concerns also apply to immobilized homogeneous populations of biologically synthesized oligomer or other nucleic acid molecules.

While the situation concerning surface immobilized oligomer molecule populations is complex, the practice of the invention can be used to obtain information and results concerning the functional homogeneity and functional characteristics of the analyzed immobilized oligomer molecules which, relative to such prior art obtained information and results, is improved. Further, the practice of the invention can also be used to obtain information and results concerning the functional homogeneity and functional characteristics of the complementary oligomer molecules which are used to analyze the immobilized oligomer molecules which, relative to such prior art obtained information and results, is improved. These improved results are obtained by applying the basic rationale and methods described for the practice of the invention to the analysis of the immobilized oligomer molecule system.

Application of Basic Invention Rationale for Determining the Functional Homogeneity and Functional Characteristic Values of Non-Nucleic Acid Chemically and Biologically Synthesized Proteins and Other Molecules.

The basic practice of the invention for determining the functional homogeneity and functional characteristic values of a molecule of interest which interacts with another molecule to form a dissociable Bi-Molecular Complex (herein termed a BMC), is to determine a dissociation kinetic profile under constant temperature and milieu, for the BMC molecule population of interest, in order to determine whether the dissociation kinetic profile has the expected first order shape or form. Earlier, this basic practice of the invention for determining the functional homogeneity of nucleic duplex BMCs was extensively discussed, and illustrated. These earlier discussions, descriptions, and illustrations described in detail the general rationale and other aspects of the basic invention. It will be apparent to one of skill in the art that the general rationale and other aspects of the basic invention can also be used to determine improved functional homogeneity and functional characteristic values for non-nucleic acid BMCs of essentially all kinds. It will also be apparent to one of skill in the art that different methods for measuring the absolute or relative amounts of BMCs and dissociated BMCs are required for different BMC types in order to practice the basic invention. It will further be apparent to one of skill in the art that different prior art methods for determining the relative or absolute amounts of dissociated and non-dissociated BMC molecules in a sample, are available for many different types of non-nucleic acid chemically and biologically synthesized and other molecules. As an example, a variety of methods are available for determining the amounts of protein·protein or protein·hapten BMCs and dissociated protein·protein or protein·hapten BMCs, which are present in a sample. These include antibody·antigen and antibody·hapten BMCs. Overall then, the basic practice of the invention is applicable to essentially all situations where dissociable BMCs are formed. Note that the basic practice of the invention is applicable for BMCs in solution or which are immobilized on a solid surface.

Similarly, the basic practice of the invention is applicable to determining improved quantitative functional homogeneity and functional characteristic values for dissociable molecular complexes consisting of 3 or more separate molecules of the same or different compositions.

Further, the basic practice of the invention can be used to determine the effect of the presence of small and large biological and/or non-biological molecules on the quantitative functional homogeneity and functional characteristic values of dissociable molecular complexes consisting of 2, or 3, or more separate molecules of the same or different composition.

Note that the basic practice of the invention can also be used to determine improved functional homogeneity and functional heterogeneity values for BMC, TMC, and other complexes composed of nucleic acid and non-nucleic acid molecules.

Invention Improved Oligomer Functional Homogeneity and Functional Characteristic Results “Improvement Ripple Effect”: Further Practices of the Present Invention.

The production of invention improved functional homogeneity and/or functional characteristic values for a nucleic acid oligomer prep causes an “improvement ripple effect”, which extends far downstream from the immediate direct use of the improved functional homogeneity and functional characteristic results in an oligomer application. The direct use of invention improved functional homogeneity and functional characteristic results for an application is termed a zero order application.

The use of the invention improved functional homogeneity and functional characteristic results in a zero order application produces zero order application results which are, relative to prior art produced zero order application results, significantly improved, and is a practice of the invention. Examples of such zero order application uses of improved functional homogeneity and functional characteristic results include, but are not limited to, the following. (a) Producing improved DNA and RNA chemical synthesis reagents, protocols, instruments, and processes. (b) Producing improved oligomer duplex equilibrium constants. (c) Producing improved oligomer primers of all kinds for the in vitro enzymatic synthesis of RNA or DNA. (d) Producing improved oligomer capture probes for gene expression analysis. (e) Producing improved oligomer DNA and RNA diagnostic probes of all kinds. (f) Producing improved SNP or mutation detection oligomer probes of all kinds. (g) Producing improved site directed mutagenesis oligomers of all kinds. (h) Producing improved oligomers for gene synthesis. (i) Producing improved oligomer siRNA and miRNA and other regulatory nucleic acid oligomers. (j) Producing improved molecular beacon, FRET, and other fluorescent oligomers of all kinds. (k) Producing improved nucleic acid sequencing oligomers of all kinds. (l) Producing improved oligomer nucleic acid standards of all kinds. (m) One of skill in the art will recognize that these zero order application examples are only a few of the large number of possible zero order applications.

A further downstream improvement ripple effect is the direct use of improved zero order application results in a further application which directly uses zero order application results. Such a further application is herein termed a first order application. The use of invention improved zero order application results in a first order application produces first order application results which are, relative to prior art produced first order application results, significantly improved, and is a practice of the present invention. Examples of such first order application use of improved zero order application results to produce improved first order application results include, but are not limited to, the following. (a) Producing chemically synthesized oligomers of all kinds which are improved in production reproducibility and quality. (b) Producing improved oligomer and oligomer duplex thermodynamic property results. (c) Producing improved oligomer primer dependent RNA and DNA enzymatic synthesis results. (d) Producing improved gene expression analysis assay results. (e) Producing improved oligomer associated biological assay results. (f) Producing improved oligomer based SNP detection assay results. (g) Producing improved oligomer based SNP directed mutagenesis results. (h) Producing oligomer based gene synthesis results. (i) Producing improved siRNA and miRNA and other regulatory nucleic acid assay results. (j) Producing improved results for assays using molecular beacons, FRET, and other fluorescent labeled oligomers. (k) Producing improved oligomer mediated nucleic acid sequencing results. (l) Producing improved results for assays using oligomer standards. (m) One of skill in the art will recognize that these first order application examples are only a few of a great many possible first order applications.

An even further downstream improvement ripple effect is the direct use of improved first order application results in an even further application which directly uses one or more first order application results. Such an application is herein termed a second order application. The use of invention improved first order application results in a second order application produces second order application results which are, relative to prior art produced second order application results, significantly improved, and is a practice of the present invention. Examples of such second order application use of improved first order application results include, but are not limited to, the follow. (i) Producing improved methods for predicting the behavior of oligomers and oligomer duplexes in an oligomer application by using improved thermodynamic property results. (ii) Producing improved results for applications which utilize improved oligomer primary dependent enzymatic synthesis methods. (iii) Producing improved data mining analysis results by using improved gene expression analysis results. (iv) Producing improved data mining analysis results by using improved SNP detection results. (v) Producing improved data mining analysis results by using improved biological assay results. (vi) Producing improved data mining analysis results by using improved siRNA, miRNA or other regulatory nucleic acid assay results. (vii) Producing improved data mining results by using improved oligomer standard assay results. (viii) Producing improved drug or other product candidate identification and validation results by using improved gene expression analysis results and/or improved SNP detection results and/or improved biological assay results and/or improved siRNA, miRNA, or other regulatory nucleic acid assay results and/or improved oligomer standard assay results. (ix) One of skill in the art will recognize that these second order application examples represent only a few of a great many possible second order applications.

Another downstream improvement ripple effect is the use of invention improved second order application results in a still further application which directly uses one or more first and/or second order application results. Such an application is herein termed a third order application. The use of invention improved second order application results, or first and second order applications results in a third order application produces third order application results which are, relative to prior art produced third order applications results, significantly improved, and is a practice of the invention. Examples of such third order applications include, but are not limited to, the following. (a) Producing improved oligomer associated assays of all kinds by using improved methods for predicting the behavior of oligomers and oligomer-target duplexes. (b) Producing improved oligomer primer dependent assays of all kinds by using improved methods for primer dependent enzymatic synthesis. (c) Producing improved systems biology analysis results by using improved data mining analysis results which incorporate improved gene expression analysis results and/or improved SNP detection results and/or improved biological assay results and/or improved siRNA, miRNA, or other regulatory nucleic acid results and/or improved oligomer standard assay results. (d) Producing improved drug or other product selection for further development results by using improved drug candidate identification and validation results and/or improved data mining and/or systems biology results. (e) One of skill in the art will recognize that these third order application examples represent only a few of a great many possible third order applications.

Higher order applications also occur which produce invention improved higher order application results which, relative to prior art higher order application results, are significantly improved and represent practices of the invention.

A large number of such higher order applications which utilize one or more improved lower order application results to produce invention improved higher order application results are possible and include, but are not limited to the following selected examples. (i) Producing improved drugs, bioactive molecule, or other product higher order application development and/or toxicology and/or safety and/or QA-QC and/or pharmacologic and/or pharmacokinetic and/or clinical study candidate selection and evaluation and/or market segment identification and/or effectiveness of use in patents and/or manufacturing results and other improved results, using one or combinations of two or more invention improved lower order application results. A more specific example is the production of improved pharmaceutical of any kind validation and toxicity and/or safety results by using improved data mining and systems biology analysis results. Another more specific example is the production of improved pharmaceutical agent patient prescription and/or drug treatment efficacy evaluation strategies and information by using invention improved toxicology and/or safety and data mining analysis and system biology analysis results and other improved results.

Computer Implementation of Methods for Determining Improved Oligomer Functional Characteristic Values, Thermodynamic Values, Oligomer Selection, and Oligomer Application Optimization

The portions of the invention involving the measurement, determination, and calculation of functional characteristic and functional homogeneity values for particular oligomers under particular conditions of solution composition, pH, temperature, pressure, and electric field strength, facilitate the determination of more accurate thermodynamic (TD) property values (such as changes in the various forms of Activation Energy[Ea], Free Energy[G], Enthalpy[H], and Entropy[S]). One of skill in the art will recognize that a data base consisting of such functional and TD values along with derived equilibrium constant values, for a plurality of different oligomers of different nucleotide or nucleotide analog sequence, nucleotide or nucleotide analog length, and nucleotide composition or nucleotide analog composition, obtained for a plurality of different conditions of solution composition, pH, temperature, pressure, and electrical field strength, can be used to produce software program or non-software program methods for calculating or determining the functional and TD parameter values for other particular oligomers under particular application conditions Persons skilled in the field are familiar with performing the relevant calculations, comparing and correlating and interpreting the resulting values, coding the functions in a suitable programming language, and configuring computers to implement the resulting programs. Thus, the calculational steps will not be repeated here. A large number of programs have been developed for performing similar functions based on the types of oligomer-related information previously available (e.g., from TD results derived from OM determinations). If desired, such software can be modified or extended to perform the present calculations.

Thus, the present invention also concerns such computer software. Such software may be in hard copy (e.g., printing code and/or data) or may be embedded in one or more forms of computer accessible data storage such as random access memory (RAM), read only memory (ROM), magnetic storage media such as computer hard drives, tapes, and floppy disks, optical storage media such as CDs and DVDs and the like, and flash memory devices. The software may be in one or more portions (e.g., modules), which may be in the same physical storage device or in a plurality of different physical storage devices. Likewise, when loaded on a computer, the software may be accessible from a single computer, from any of multiple computers on a LAN or other local network or file transfer connection, or from any of multiple computers over the internet or a WAN or other large scale network. Therefore, the invention also concerns data storage devices and computer systems in which such software is loaded or stored, as well as methods using such software and computer systems to perform the designed functions of the software.

Such functions and methods (and the associated software and computer systems) can, for example, involve in silico design or selection of oligomers for a particular oligomer application and application condition, methods for selecting one or more oligomers for a particular application and application condition, calculation of oligomer functional characteristic values under a particular application condition and parameters derived therefrom and/or TD values for molecular processes such as hybridization and dissociation under a particular application condition, and matching of such values with oligomer application requirements and specifications and conditions.

The various functions can be performed by separate software programs or other methods, or can be embodied in a single software program or other method. As indicated, one useful software function (or program) is the calculation of functional and thermodynamic values (TD) for any particular wanted or unwanted candidate oligomer-target duplex for a particular application and condition. Such calculations can involve what is essentially a look-up table to find corresponding experimentally determined values for the closest matches of oligomer and conditions, and then to interpolate to determine predicted values for the particular oligomer. Alternatively, or as part of the refinement, an algorithm based on such experimentally determined values (and/or underlying functional characteristic values) is used to calculate predicted values. Further, in a basic embodiment, the functional and TD values are calculated for a particular oligomer preparation based on measured functional characteristic values for that preparation.

Another useful software function (or program) provides identification from available nucleic acid sequence data bases or de novo, of candidate effective oligomers (or predicted optimized oligomers) for a particular oligomer application and condition. For this software, the oligomer application and/or application conditions and requirements, and/or oligomer requirements are specified. Such specification can be performed in several ways. For example, specification of the particular application may, within the program or associated databases or tables, calculate or look-up specifications for application conditions and/or oligomer requirements. Alternatively or in addition, such conditions and/or application or oligomer requirements may be input by the user. The software calculates predicted performance for specified oligomers and/or creates and tests oligomers to identify oligomers which are predicted to provide effective or even optimized performance in the application.

Another function (or program) evaluates the effect of damaged oligos in an oligomer preparation. This may be performed separately, but can advantageously be combined with the preceding function. As with the preceding function, the oligomer application is specified, along with application conditions and requirements and/or oligomer requirements. The software calculates the effects of damaged nucleotide sites in the oligomer population on performance of the oligomer prep in the application. Such calculations can use look-up tables or similar data compilations and/or predictive algorithms. Typically, such data compilations and/or predictive algorithms contain or are based on experimental results for the performance of actual characterized oligomer preparations.

As indicated above, the oligomer application, and generally application conditions and requirements and/or oligomer requirements are specified. The particular specifications will depend on the particular application. Thus, one parameter which may be specified is the function for the oligomer in the application, e.g., as a primer, hybridization probe, capture probe, or ligation oligomer,

Specification of at least some of the application conditions are important. Parameters for such conditions can include, for example, hybridization conditions (e.g., temperature, solution composition and conditions, free or immobilized oligomer, oligomer concentration, length of hybridization interval), wash conditions (e.g., temperature, solution conditions, free or immobilized oligomer, flow rate, length of wash interval), and reading conditions (type of signal to be read, temperature, solution conditions).

Further, it is useful to specify requirements for the oligomers involved in the application. Such requirements, may include, for example, whether a high ka is required, the intended oligomer sequence, the intended target sequence, non-intended target sequences, intended oligomer-target duplex stability, oligomer-non-target duplex stability, and the state of the oligomer-target duplex in the application (e.g., stable duplexes such that essentially all duplexes will remain hybridized through the application, or in equilibrium such that a significant fraction of the duplexes will dissociate during the application.

### CONCLUSION

For the purpose of explanation the foregoing discussions used specific nomenclature to provide a thorough understanding of the invention and its many embodiments. However, it will be apparent to one of skill in the art that this nomenclature and description are but one way to describe the invention and its mode of practice. Thus, the foregoing nomenclature and description are presented for the purpose of illustration and description, and they are not intended to be exhaustive or to limit the invention to the precise forms disclosed, and obviously many modifications and variations are possible as a result of the above teachings. The discussions presented were selected and described in order to best explain the present invention and its practical applications, and to thereby enable others skilled in the art to best practice the invention and various embodiments with various modifications, as are suited to the particular use contemplated.

All publications and patents or patent applications cited in this specification are herein incorporated by reference as if each individual publication or patent application were specifically and individually indicated by reference. The citation of any publication for its disclosure prior to the filing date should not be construed as an admission that the present invention is not entitled to antedate such publication by virtue of prior invention.

Abbreviations used in the claims are defined in the body of this description.

### REFERENCES CITED

(1) Agrawal, Protocols for Oligonucleotides and Analogs. Synthesis and Properties. Methods in Mol. Bio. Vol. 20. Humana Press (2003).
(2) Herdewjin, Oligonucleotide Synthesis. Methods and Applications. Methods in Mol. Bio. Vol. 288. Humana Press (2005).
(3) Khudyakov et al. Artificial DNA. Methods and Applications. CRC (2003).
(4) Demidov et al. DNA Amplification. Current Technologies and Applications. Horizon Bioscience (2004).
(5) Agrawal. Protocols for Oligonucleotides Conjugates. Synthesis and Analytical Techniques. Methods Mol. Bio. Vol. 26. Humana Press (1994).
(6) Temsamani et al. Sequence Identity of the N−1 Product of a Synthetic Oligonucleotide. Nuc. Acid. Res. 23(11): 1841-1844 (1995).
(7) Chen et al. Analysis of Internal N−1 Deletion Sequences in Synthetic Oligodeoxyribonucleotides By Hybridization To An Immobilized Probe Array. Nuc. Acid. Res. 27(2): 389-395 (1999).
(8) Fearon et al. Investigation of the N−1 Impurity in Phosphorothioate Oligodeoxynucleotides Synthesized By the Solid—Phase B—Cyanoethyl Phosphoramidite Methods Using Stepwise Sufurization. Nuc. Acid. Res. 23(14):2754-2761 (1995).
(9) Farrance et al. Improved Chemistry for Oligodeoxyribonucleotide Synthesis Substantially Improves Restriction Enzyme Cleavage of a Synthetic 35mer. Nuc. Acid Res. 17(3): 1231-1245 (1989).
(10) Hecker et al. Error Analysis of Chemically Synthesized Polynucleotides. Biotechniques. 24(2): 256 (1998).
(11) Foldes-Papp et al. Efficiency of Chemical Oligonucleotide Synthesis Evaluated by Ion-Exchange HPLC. J. Chromatog. A. 706: 405-419 (1995).
(12) Mitchelson et al. Capillary Electrophoresis of Nucleic Acids. Introduction. Vol. 1. Methods in Mol. Bio. Vol. 162. Humana Press (2001).
(13) Mitchelson et al. Capillary Electrophoresis of Nucleic Acids. Practical Applications. Vol. II. Methods in Mol. Bio. Vol. 163. Humana Press (2001).
(14) Marky et al. Calculating Thermodynamic Data for Transitions of Any Molecularity from Equilibrium Melting Curves. Biopolymers. 26:1601-1620 (1987).
(15) Wetmur. DNA Probes: Applications of the Principles of Nucleic Acid Hybridization. Critical Reviews in BCHM. and Mol. Bio. 26(3/4): 227-259 (1991).
(16) Breslauer. In Thermodynamic Data for Biochm. and Biotech. H. Hinz, editor. Springer-Verlag, New York. Page 402-437 (1986).
(17) Owczarzy et al. Effects of Sodium Ions on DNA Duplex Oligomers: Improved Predictions of Melting Temperatures. Biochemistry. 43:3537-3554 (2004).
(18) Remond et al. ROSO: Optimizing Oligonucleotide Probes for Microarrays. Bioinformatics. 20(2): 271-273 (2004).
(19) Gordon. Osprey: A Comprehensive Tool Employing Novel Methods for the Design of Oligonucleotides for DNA Sequencing and Microarrays. Nuc. Acid Res. 32(17):e133.
(20) Leontis et al. Molecular Modeling of Nucleic Acids. ACS Symposium Series 682. ACS Book Dept. (1998).
(21) Porschke et al. Thermodynamics and Kinetics of the Helix-Coil Transition of Oligomers Containing GC Base Pairs. Biopolymers. 12:1313-1335 (1973).
(22) Nelson et al. Comparison of Kinetics of Ribooligonucleotide, Deoxyribonucleotide, and Hybrid Oligonucleotide Double-Strand Formation By Temperature-Jump Kinetics. Biochemistry. 21:5289-5295 (1982).
(23) Williams et al. Laser Temperature-Jump Spectroscopic, and Thermodynamic Study of Salt Effects on Duplex Formation By dGCATGC. Biochemistry. 28(10): 4283-4291 (1989).
(24) Morrison et al. Sensitive Fluorescence—Based Thermodynamic and Kinetic Measurements of DNA Hybridization in Solution. Biochemistry. 32:3095-3104 (1993).
(25) Nelson et al. DNA and RNA Oligomer Thermodynamics: The Effect of Mismatched Bases on Double-Helix Stability. Biopolymers. 20: 2509-2531 (1981).
(26) Young et al. Hybridization and Dissociation Rates of Phosphodiester or Modified Oligodeoxyribonucleotides with RNA at Near Physiological Conditions. Nuc. Acid. Res. 19(9): 2463-2470 (1991).
(27) Ikuta et al. Dissociation Kinetics of 19 Base Paired Oligonucleotide-DNA Duplexes Containing Different Single Mismatched Base Pairs. Nuc. Acid Res. 15(2): 797-811 (1987).
(28) Lorkowski et al. Analysing Gene Expression. A Handbook of Method Possibilities and Pitfalls. Vol. 1 and 2. Wiley-VCH (2003).
(29) Hammes et al. Nucleic Acid Hybridization. A Practical Approach. IRL Press Ltd. (1985).
(30) Kohne et al. Hydroxapatite Techniques for Nucleic Acid Reassociation in Procedures in Nucleic Acid Research Vol. 2. Cantoni et al. Eds. Harper and Row (1971).
(31) Caccone et al. Calibration of the Change in Thermal Stability of DNA Duplexes and Degree of Base Pair Mismatch. J. Molec. Evol. 27:212-216 (1988).
(32) Porschke et al. Co-Operative Non-Enzymatic Base Recognition: III Kinetics of the Helix-Coil Transition of the Oligoribouridylic. Riboadenylic Acid System and of Oligoriboadenylic Acid Alone At Acidic pH. J. Mol. Bio. 62:361-381 (1971).
(33) Tracy et al. Detection, Sizing and Quantitation of Polyadenylated RNA in the Nanogram-Picogram Range. Biochemistry. 19:3792 (1980).
(34) Rapely. The Nucleic Acid Protocols Handbook. Chapt. 19. Humana Press (2000).
(35) Horrocks. Applications of Liquid Scintillation Counting. Chapt. 14. Academic Press. New York. (1974).
(36) Kwok, P. Methods in Molecular Biology. Vol. 212, Single Nucleotide Polymorphisms, Methods and Protocols. Humana Press (2003)
(37) Edwards et al. Real Time PCR, An Essential Guide. Horizon Bioscience Norfolk, UK (2004)

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of presently preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the techniques used for determining oligomer preparation homogeneity and other characteristics. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

1. A method for obtaining improved information or results or both concerning the functional homogeneity of the population of the oligomer molecules which are present in a preparation of chemically synthesized or in vitro enzymatically synthesized or biologically synthesized oligomer preparations, comprising determining one or more of the following for the oligomer preparation:
(a) the FH value for the oligomer prep under analysis conditions of interest;
(b) the DK profile for the oligomer prep under analysis conditions of interest;
(c) the % SF and % FF values for the oligomer prep under analysis conditions of interest;
(d) the SF and FF t.5d values for the oligomer prep under analysis conditions of interest;
(e) the pattern of nucleotide sequence damage associated with the oligomer preparation's FF; and
(f) the extent of nucleotide sequence damage associated with the oligomer preparation's FF.

2. A method for obtaining improved information or results or both concerning the functional characteristics of the oligomer molecules which are present in a preparation of chemically synthesized or in vivo enzymatically synthesized or biologically synthesized oligomer preparations, comprising determining for the oligomer preparation one or more of the following:
(a) the FH value for the total oligomer preparation under analysis conditions of interest;
(b) the DK profile for the total oligomer prep under analysis conditions of interest;
(c) the % SF and % FF values for the total oligomer prep under analysis conditions of interest;
(d) the SF t.5d and FF t.5d values under the analysis conditions of interest;
(e) the pattern of nucleotide sequence damage associated with the oligomer FF; and
(f) the extent of nucleotide sequence damage associated with the oligomer FF.

3. The method of claim 2, further comprising determining the hybridization kinetic association constant kₐ value or values for the total oligomer prep under the analysis conditions of interest.

4-8. (canceled)

9. A method for improving oligomer application results, comprising
improving the functional effectiveness of an oligomer application utilizing an oligomer prep by
(a) Determining a plurality of the oligomer prep functional characteristic values for FH, % SF and % FF, SF t.5d, FF t.5d, pattern of FF nucleotide sequence damage, extent of FF nucleotide sequence damage, and hybridization kinetic Kₐ under one or more application conditions of interest, for one or more oligomer preps which are designed and produced for an oligomer application;
(b) quantitating or otherwise determining the functional effectiveness of each oligomer prep in the oligomer in the application of interest, and quantitating or otherwise determining whether the actual functional effectiveness of the application is the intended functional or desired effectiveness;
(c) correlating the functional characteristic values for each oligomer prep with the oligomer prep's functional effectiveness value, in order to identify an improved useful or optimal set of oligomer functional characteristics for the intended oligomer application; and
(d) using the identified oligomer prep for the intended application to obtain improved oligomer application results.

10. The method of claim 9, wherein the intended oligomer application comprises one of more of the following:
An RT-PCR or PCR application;
An enzymatic synthesis of RNA or DNA application;
A nucleic acid synthesis primer application;
A nucleic acid sequencing application;
A gene cloning application;
A gene expression analysis or gene expression comparison analysis application;
A nucleic acid hybridization application;
A DNA and/or RNA mutation detection or SNP detection application;
A method of calorimetry analysis for determining oligomer physical-chemical and thermodynamic information application;
A determination of oligomer Tm values by OM analysis application;
A determination of oligomer duplex equilibrium constant by OM or other methods analysis application;
The use of an oligomer as a standard for an oligomer application or other application;
A nucleic acid ligation application;
A fluorescent labeled oligomer application;
A radioactive labeled oligomer application;
A chemiluminescent labeled oligomer application;
An enzyme labeled oligomer application;
A metal or non-metal nano particle label oligomer application;
A hybridized duplex strand displacement application;
A biotin labeled oligomer application;
A ligand molecule labeled oligomer application;
A receptor or binding molecule application;
An application using a chemically modified oligomer;
A use of modified or unmodified oligomers to form nano-structures or patterns or functions applications;
A DNA or RNA oligomer of any kind application;
An antisense or aptamer DNA or RNA application;
A pharmaceutical antibiotic, anti-viral, or therapeutical or vaccine application;
A gene synthesis application;
A regulatory siRNA, miRNA, or RNA or DNA gene expression suppression or enhancement application;
A site directed mutagenesis application;
A discovery and identification of expressed particular gene(s) and gene expression profile(s) which are characteristic of one of more particular normal state(s) or disease state(s) application.

11-19. (canceled)

20. A method for improving functional effectiveness of oligomer preparations prepared using a particular synthesis method, comprising
Determining at least one of the oligomer prep functional characteristic values FH, % SF and % FF, SF t.5d, FF t.5d, pattern of FF nucleotide sequence damage, extent of FF nucleotide sequence damage, and hybridization kinetic kₐ under one or more conditions, for a plurality of preps selected for potential use in said oligomer application;
Selecting an oligomer prep from said plurality of oligomer preps having better functional characteristic values for said oligomer application.

21-22. (canceled)

23. The method of claim 20, wherein said functional effectiveness corresponds to functional homogeneity of said oligomer prep.

24. The method of claim 20, wherein said functional effectiveness corresponds to a functional characteristic of said oligomer prep.

25. The method of claim 20, wherein the intended oligomer application comprises one of more of the following:
An RT-PCR or PCR applications;
An enzymatic synthesis of RNA or DNA application;
A nucleic acid synthesis primer application;
A nucleic acid sequencing application;
A gene cloning application;
A gene expression analysis or gene expression comparison analysis application;
A nucleic acid hybridization application;
A DNA and/or RNA mutation detection or SNP detection application,
A method of calorimetry analysis for determining oligomer physical-chemical and thermodynamic information application;
A determination of oligomer Tm values by OM analysis application;
A determination of oligomer duplex equilibrium constants by OM or other methods analysis application;
The use of an oligomer as a standard for an oligomer application or other application;
A nucleic acid ligation application;
A fluorescent labeled oligomer application;
A radioactive labeled oligomer application;
A chemiluminescent labeled oligomer application;
An enzyme labeled oligomer application;
A metal or non-metal nano particle label oligomer application;
A hybridized duplex strand displacement application;
A biotin labeled oligomer application;
A ligand molecule labeled oligomer application;
A receptor or binding molecule application;
An application using a chemically modified oligomer;
A use of modified or unmodified oligomers to form nano-structure or patterns or functions application,
A DNA or RNA oligomer of any application;
An antisense or aptamer DNA or RNA application;
A pharmaceutical antibiotic, anti-viral, or therapeutic or vaccine application;
A gene synthesis application;
A regulatory siRNA, miRNA, or RNA or DNA gene expression suppression or enhancement application;
A site directed mutagenesis application;
A discovery and identification of expressed particular gene(s) and gene expression profile(s) which are characteristic of one of more particular normal state(s) or disease state(s) application.

26. The method of claim 20, wherein a plurality of said oligomer preps are synthesized using a plurality of different synthesis methods.

27. The method of claim 26, wherein said plurality of different synthesis methods comprises a plurality of modifications of one synthesis method.

28. The method of claim 27, wherein in said plurality of modifications of one synthesis method, one or more of said functional characteristics of said oligomer preps produced from said plurality of modifications of one synthesis method are compared to select an optimized synthesis method producing improved oligomer preps for said oligomer application.

29-39. (canceled)

40. A method for producing improved information and results for a zero order application which directly utilizes measured oligomer and oligomer preparation functional homogeneity and functional characteristic results, comprising,
using the methods of claim 1 to produce improved oligomer and oligomer preparation functional homogeneity and functional characteristic information and results; and
utilizing a part or all of said improved oligomer and oligomer preparation functional homogeneity information and results in a zero order application, thereby producing one or more improved zero order application information and result.

41. The method of claim 40, wherein said zero order application comprises one or more of the following:
A method for producing DNA, RNA, or modified RNA or DNA synthesis reagents;
A method for producing oligomer duplex of any kind equilibrium constants;
A method for producing oligomer primers of all kinds for the in vitro enzymatic synthesis of RNA or DNA;
A method for producing oligomer for use in a nucleic acid ligation process;
A method for producing oligomer capture probes for gene expression analysis;
A method for producing oligomer RNA or DNA diagnostic probes;
A method for producing SNP and base pair mismatch detection oligomers;
A method for producing site directed mutagenesis oligomers;
A method for producing oligomers for use in gene synthesis;
A method for producing oligomer siRNA and miRNAs and other regulatory RNAs;
A method for producing molecular beacon, FRET, and other fluorescent molecule associated oligomers;
A method for producing oligomers for use in nucleic acid sequencing;
A method for producing improved oligomer assay and other standard; and
A method for producing synthesis processes, protocols, and QA/QC procedures.

42-47. (canceled)

48. A method for producing improved information and results for a higher order application, which directly utilizes one or more lower application results, comprising,
(a) Using the method of claim 1 to produce improved lower order application information and results; and
(b) Utilizing all or part of said improved lower order information and results in a higher order application, thereby producing one or more improved higher order application results.

49. The method of claim 48, wherein said lower order application comprises one or more of the following:
(a) A zero order application;
(b) A first order application;
(c) A second order application;
(d) A third order application; and
(e) A higher than third order application.

50. The method of claim 49, wherein said higher order application comprises one or more of the following:
(a) One or more methods for producing drug or bioactive molecule or biomarker clinical study candidate selection results;
(b) One or more methods for producing drug or bioactive molecule or biomarker clinical study evaluation results;
(c) One or more methods for producing drug or bioactive molecule or biomarker manufacturing and QC/QA results;
(d) One or more methods for producing drug or bioactive molecule or biomarker or other product market segment selection process results;
(e) One or more methods for producing drug or bioactive molecule or biomarker or other product prescription and use in the patient results;
(f) One or more methods for producing drug or bioactive molecule or biomarker efficacy in the patient;
(g) One or more methods for producing drug bioactive molecule or biomarker or other product toxicological characteristic results;
(h) One or more methods for producing disease or pathology state prognosis prediction results; and
(i) One or more methods for producing disease or pathology state prognosis prediction after drug or bioactive molecule or other product treatment.

51. A characterized oligomer preparation, comprising
an oligomer preparation; and
a data set embedded in a hardcopy, computer display, or electronic data storage medium describing one or more characteristics of said oligomer preparation, wherein at least some of said data set comprises
improved oligomer functional homogeneity information or results or both; or
improved functional characteristic information or results or both,
wherein said information and results or both are produced by the method of claim 1.

52-53. (canceled)

54. The method of claim 51, wherein said information and results comprises at least one of functional characteristic values for FH, % SF and % FF, SF t.5d, FF t.5d, pattern of FF nucleotide sequence damage, extent of FF nucleotide sequence damage, and hybridization kinetic kₐ corresponding to said oligomer preparation under one or more conditions of interest.

55-63. (canceled)

64. A data set at least partially describing characteristics of at least one oligomer prep, comprising
a data set specifying one or more characteristics of said oligomer prep comprising data corresponding to at least one of
oligomer functional homogeneity, and
oligomer functional characteristics,
wherein said data set is embedded in a hard copy, computer display, or electronic data storage medium.

65-67. (canceled)

68. The data set of claim 64, wherein said data set includes data at least partially describing improved functional homogeneity and improved functional characteristic values obtained under one or more know conditions of solution composition, pH, temperature, pressure, and electric field strength, for each of a plurality of different oligomers which differ in nucleotide sequence and/or nucleotide length and/or nucleotide composition.

69. The data set of claim 64, wherein said data set includes data describing at least one of FH, % SF and % FF, SF t.5d, FF t.5d, pattern of FF nucleotide sequence damage, extent of FF nucleotide sequence damage, hybridization kinetic kₐ for said oligomer preparation.

70-83. (canceled)

84. A method for selecting one or more particular oligomers for use in an oligomer application, comprising
identifying a particular oligomer based on having at least one of improved functional homogeneity, functional characteristics, or improved thermodynamic property values appropriate for an intended oligomer application.

85. The method of claim 84, wherein functional homogeneity, functional characteristics, or improved thermodynamic property values are determined according to claim 1.

86. The method of claim 84, further comprising
determining application conditions which are compatible with said application;
identifying at least one oligomer having or expected to have desired functional homogeneity and functional characteristics under said application conditions.

87-105. (canceled)
